(19) United States
(12) Patent Application Publication
(10) Pub. No.: US 2026/0221053 A1
(43) Pub. Date: Jul. 30, 2026

(54) DRUG DELIVERY DEVICES, SAFETY SYSTEMS, TRAINING DEVICES, AND METHODS OF USE THEREOF

(71) Applicant: Regeneron Pharmaceuticals, Inc., Tarrytown, NY (US)

(72) Inventors: Trevor LANGLEY, Rensselaer, NY (US); Connor DOHERTY, Albany, NY (US); Jessica MIKLINSKI, Cohoes, NY (US)

(73) Assignee: Regeneron Pharmaceuticals, Inc., Tarrytown, NY (US)

(21) Appl. No.: 19/460,903

(22) Filed: Jan. 27, 2026

Related U.S. Application Data
(60) Provisional application No. 63/750,881, filed on Jan. 29, 2025.
(60) Provisional application No. 63/798,223, filed on May 1, 2025.
(60) Provisional application No. 63/919,811, filed on Nov. 18, 2025.

(51) Int. Cl. G09B 23/28; A61M 5/20; A61M 5/31; A61M 5/315; A61M 5/32

(52) U.S. Cl. CPC G09B 23/285; A61M 5/2033; A61M 5/3137; A61M 5/31501; A61M 5/3157; A61M 5/3213; A61M 2005/31508; A61M 2205/581

(57) ABSTRACT

Described herein are training devices and methods of using the training devices. In particular, disclosed herein are training devices that simulate the experience of using an injection device for administering drugs to patients. The training devices may include lockout mechanisms simulating similar mechanisms in injection devices. Unlike injection devices, the training devices may be resettable, with reset mechanisms that provide for the bypass of the lockout mechanisms, thereby allowing for the reuse of the training device.

### CROSS-REFERENCE TO RELATED APPLICATIONS

This application claims the benefit of priority to U.S. Provisional Patent Application No. 63/919,811 filed on Nov. 18, 2025, U.S. Provisional Patent Application No. 63/798,223 filed on May 1, 2025, and U.S. Provisional Patent Application No. 63/750,881 filed on Jan. 29, 2025, each of which is incorporated by reference herein.

### TECHNICAL FIELD

This disclosure is directed to training devices simulating drug delivery devices (e.g., prefilled syringes), safety systems, and uses thereof.

### INTRODUCTION

Drug delivery devices (e.g., prefilled syringes, auto-injectors, or other suitable drug delivery devices) are routinely used to deliver fluid drug substances. Various safety concerns may arise before, during, and after the use of a drug delivery device. To address such concerns, conventional safety devices have been developed. Conventional safety devices may include an active safety mechanism. In such devices, the user is required to actively perform a specific action to trigger a safety mechanism. It may be difficult for a user to properly activate a safety mechanism, while at the same time, properly utilizing the drug delivery device for injecting a drug substance into themselves or another person. In contrast to these active safety devices, passive safety devices have been developed, where there is no need for a user to perform any additional and/or separate action to trigger a safety mechanism during and/or after use of a drug delivery device.

In passive safety systems, there may still be concerns for user safety and proper usage of the drug delivery device. For example, there may be premature expulsion of the fluid, accidental needle stick pre-injection and/or post-injection, and/or premature lockout prior to end of dose. Needle anxiety, inexperience, and/or lack of confidence during use of such devices may contribute to lack of safety and improper usage of the drug delivery devices.

Training devices including needle simulation and operation technology allow for replication of a treatment experience, alleviating anxiety and improving confidence, before a user operates a drug delivery device. Training devices may therefore provide improved safety as well as more accurate dosage delivery when using non-training drug delivery devices. Training devices replicating features of the passive safety systems are particularly useful, especially when such devices also include reset features allowing for repeated practice.

### SUMMARY OF THE DISCLOSURE

The present disclosure describes a training device comprising a first housing; a second housing partially housed within the first housing, wherein the second housing comprises a proximal end comprising a plurality of wings and a distal end opposite to the proximal end, wherein each of the plurality of wings comprises a chamfered proximal surface; a primary container housed within the second housing; a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface; and a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces.

Various embodiments of the device may include one or more of the following aspects. The device may include a lockout mechanism comprising a proximal portion of the plunger rod configured to receive the plurality of tabs and contact the filleted inner surfaces of the plurality of tabs when the plunger rod is in a maximal distal position in which the pad of the plunger rod contacts the collar of the rear cap. Contact between the proximal portion of the plunger rod and the filleted inner surfaces of the plurality of tabs may prevent proximal movement of the plunger rod.

The device may include a reset mechanism comprising: the chamfered proximal surfaces of the plurality of wings of the second housing, wherein the chamfered proximal surfaces are configured to contact the chamfered outer surfaces of the plurality of arms and apply a inward-directing force to the plurality of arms when the second housing is in a maximal proximal position, wherein contact between the chamfered proximal surfaces of the plurality of wings and the chamfered outer surfaces of the plurality of arms allow proximal movement of the plunger rod; and a distal portion of the plunger rod configured to contact the filleted inner surfaces of the plurality of tabs and apply an outward-directing force to the plurality of tabs when the plunger rod is in a maximal proximal position.

The primary container may include a tip; an imitation needle; and a spring. The imitation needle may be attached to the spring, the spring may be housed in the tip, and the tip may be attached to the primary container. Contact between the imitation needle and an external surface may cause compression of the spring and retraction of the imitation needle into the tip. The tip may be attached to the primary container via a snap-on connection, threaded connection, or adhesive connection.

The distal end of the plunger rod comprises a cylindrical protrusion attached to a piston housed in the primary container. Contact between the piston and the primary container may generate a glide force. The piston may be attached to the cylindrical protrusion via a press-fit, threaded, or adhesive connection.

The device may include a cap configured to receive the distal end of the second housing. The device may include a plurality of extrusions on a distal surface of the pad, and a plurality of slots on the collar of the rear cap. The plurality of slots of the collar of the rear cap may be configured to receive the plurality of extrusions on the distal surface of the pad of the plunger rod. An interaction between the plurality of extrusions of the pad and the plurality of slots of the collar may maintain an alignment of the plunger rod and the rear cap. The plurality of extrusions of the pad may inhibit maximum distal movement of the plunger rod when the plurality of extrusions are not received in the plurality of slots of the collar.

The plunger rod may inhibit movement of the second housing when the plunger rod is at a maximum proximal position. The plurality of tabs may inhibit movement of the plunger rod when the plunger rod is at a maximum distal position. The second housing, the primary container, and/or the tip may be transparent.

The present disclosure also describes a method of using a training device as described herein, the method comprising: contacting the primary container to a surface; applying a force to the pad of the plunger rod to distally move the plunger rod until the pad contacts the collar of the rear cap; applying a force to the second housing to proximally move the second housing until chamfered proximal surfaces of the plurality of wings contact the chamfered outer surfaces of the plurality of arms and the second housing is in a maximal proximal position; and applying a force to the pad of the plunger rod to proximally move the plunger rod until a distal portion of the plunger rod contacts the filleted inner surfaces of the plurality of tabs and the plunger rod is in a maximal proximal position.

The present disclosure also describes a training device comprising: a first housing; a second housing partially housed within the first housing, wherein the second housing comprises a proximal end comprising a plurality of wings and a distal end opposite to the proximal end, wherein each of the plurality of wings comprises a chamfered proximal surface; a primary container housed within the second housing; a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface; and a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces; a lockout mechanism configured to prevent proximal movement of the plunger rod when the plunger rod is in a maximal distal position; and a reset mechanism configured to allow proximal movement of the plunger rod when the second housing is in a maximal proximal position.

### BRIEF DESCRIPTION OF THE FIGURES

The accompanying drawings, which are incorporated in and constitute a part of this specification, illustrate various examples and, together with the description, serve to explain the principles of the disclosed examples and embodiments.

Aspects of the disclosure may be implemented in connection with embodiments illustrated in the attached drawings. These drawings show different aspects of the present disclosure. Where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

Moreover, there are many embodiments described and illustrated herein. The present disclosure is not limited to any single aspect or embodiment thereof, and is not limited to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended to reflect or indicate the embodiment(s) is/are “example” embodiment(s).

The term “distal end” or any variation thereof, refers to the portion of the device that is the drug delivery end. Conversely, the term “proximal end” or any variation thereof, refers to the portion of the device that is the actuation end. Further, as used herein, the terms “about,” “substantially,” and “approximately” generally mean +/−10% of the indicated value.

Notably, for simplicity and clarity of illustration, certain aspects of the figures depict the general structure and/or manner of construction of the various embodiments. Descriptions and details of well-known features and techniques may be omitted to avoid unnecessarily obscuring other features. Elements in the figures are not necessarily drawn to scale; the dimensions of some features may be exaggerated relative to other elements to improve understanding of the example embodiments. For example, one of ordinary skill in the art would appreciate that the side views are not drawn to scale and should not be viewed as representing proportional relationships between different components. The side views are provided to help illustrate the various components of the depicted assembly, and to show their relative positioning to one another.

FIGS. 1A-1B depict an exemplary safety device and components thereof, according to embodiments of the present disclosure.

FIGS. 2A-2F depict additional aspects and embodiments of the exemplary safety device of FIGS. 1A-1B.

FIGS. 3A-3B depict an exemplary safety device and components thereof, according to embodiments of the present disclosure.

FIGS. 4A-4D depict additional aspects and embodiments of the exemplary safety device of FIGS. 3A-3B.

FIGS. 5A-5D depict additional aspects and embodiments of the exemplary safety device of FIGS. 3A-3B.

FIGS. 6A-6B depict an exemplary safety device and components thereof, according to embodiments of the present disclosure.

FIGS. 7A-7F depict additional aspects and embodiments of the exemplary safety device of FIGS. 6A-6B.

FIGS. 8A-8B depict an exemplary safety device and components thereof, according to embodiments of the present disclosure.

FIGS. 9A-9D depict additional aspects and embodiments of the exemplary safety device of FIGS. 8A-8B.

FIGS. 10A-10B depict additional aspects and embodiments of the exemplary safety device of FIGS. 8A-8B.

FIG. 11 depicts an exemplary safety device and components thereof, according to embodiments of the present disclosure.

FIGS. 12A-12C depict additional aspects and embodiments of the exemplary safety device of FIG. 11.

FIGS. 13A-13B depict additional aspects and embodiments of the exemplary safety device of FIG. 11.

FIGS. 14A-14C depict an exemplary safety device and components thereof, according to embodiments of the present disclosure.

FIGS. 15A-15D depict additional aspects and embodiments of the exemplary safety device of FIGS. 14A-14C.

FIGS. 16A-16B depict additional aspects and embodiments of the exemplary safety device of FIGS. 14A-14C.

FIGS. 17A-17G depict an exemplary safety device and components thereof, according to embodiments of the present disclosure.

FIGS. 18A-18B depict an exemplary safety device and components thereof, according to embodiments of the present disclosure.

FIGS. 19A-19B depict additional aspects and embodiments of the exemplary safety device of FIGS. 18A-18B.

FIGS. 20A-20C depict additional aspects and embodiments of the exemplary safety device of FIGS. 18A-18B.

FIGS. 21A-21C depict additional aspects and embodiments of the exemplary safety device of FIGS. 18A-18B.

FIGS. 22A-22B depict additional aspects and components of exemplary safety devices, according to embodiments of the present disclosure.

FIGS. 23A-23B depict an exemplary safety device and components thereof, according to embodiments of the present disclosure.

FIGS. 24A-24C depict an exemplary safety device and components thereof, according to embodiments of the present disclosure.

FIG. 25 depicts an exemplary safety device and components thereof, according to embodiments of the present disclosure.

FIGS. 26A-26B depict an exemplary safety device and components thereof, according to embodiments of the present disclosure.

FIGS. 27A-27B depict an exemplary safety device and components thereof, according to embodiments of the present disclosure.

FIGS. 28A-28B depict an exemplary safety device and components thereof, according to embodiments of the present disclosure.

FIGS. 29A-29B depict an exemplary safety device and components thereof, according to embodiments of the present disclosure.

FIGS. 30A-30D depict an exemplary safety device and components thereof, according to embodiments of the present disclosure.

FIG. 31 depicts an exemplary safety device and components thereof, according to embodiments of the present disclosure.

FIGS. 32A-32F depict an exemplary safety device and components thereof, according to embodiments of the present disclosure.

FIGS. 33A-33B depict an exemplary safety device and components thereof, according to embodiments of the present disclosure.

FIG. 34 depicts additional aspects and embodiments of the exemplary safety device of FIGS. 33A-33B.

FIGS. 35A-35B depict additional aspects and embodiments of the exemplary safety device of FIGS. 33A-33B.

FIGS. 36A-36F depict additional aspects and embodiments of the exemplary safety device of FIGS. 33A-33B.

FIGS. 37A-37D depict additional aspects and embodiments of the exemplary safety device of FIGS. 33A-33B.

FIGS. 38A-38B depict additional aspects and embodiments of the exemplary safety device of FIGS. 33A-33B.

FIGS. 39A-39C depict additional aspects and embodiments of the exemplary safety device of FIGS. 33A-33B.

FIGS. 40A-40C depict additional aspects and embodiments of the exemplary safety device of FIGS. 33A-33B.

FIGS. 41A-41B depict additional aspects and embodiments of the exemplary safety device of FIGS. 33A-33B.

FIGS. 42A-42D depict additional aspects and embodiments of the exemplary safety device of FIGS. 33A-33B.

FIGS. 43A-43B depict an exemplary safety device and components thereof, according to embodiments of the present disclosure.

FIGS. 44A-44C depict additional aspects and embodiments of the exemplary safety device of FIGS. 43A-43B.

FIGS. 45A-45C depict additional aspects and embodiments of the exemplary safety device of FIGS. 43A-43B.

FIG. 46 depicts additional aspects and embodiments of the exemplary safety device of FIGS. 43A-43B.

FIG. 47 depicts additional aspects and embodiments of the exemplary safety device of FIGS. 43A-43B.

FIGS. 48A-48B depict additional aspects and embodiments of the exemplary safety device of FIGS. 43A-43B.

FIGS. 49-51 depict additional aspects and embodiments of the exemplary safety device of FIGS. 43A-43B.

FIGS. 52A-52B depict additional aspects and embodiments of the exemplary safety device of FIGS. 43A-43B.

FIGS. 53A-53C depict additional aspects and embodiments of the exemplary safety device of FIGS. 43A-43B.

FIGS. 54A-54B depict additional aspects and embodiments of the exemplary safety device of FIGS. 43A-43B.

FIGS. 55A-55B depict additional aspects and embodiments of the exemplary safety device of FIGS. 43A-43B.

FIGS. 56A-56B depict additional aspects and embodiments of the exemplary safety device of FIGS. 43A-43B.

FIGS. 57A-57B depict additional aspects and embodiments of the exemplary safety device of FIGS. 43A-43B.

FIGS. 58A-58B depict additional aspects and embodiments of the exemplary safety device of FIGS. 43A-43B.

FIGS. 59A-59B depict additional aspects and embodiments of the exemplary safety device of FIGS. 43A-43B.

FIGS. 60A-60B depict additional aspects and embodiments of the exemplary safety device of FIGS. 43A-43B.

FIG. 61 depicts additional aspects and embodiments of the exemplary safety device of FIGS. 43A-43B in a testing environment.

FIG. 62 depicts an exemplary training device, according to aspects of the present disclosure.

FIGS. 63A-63B depict an exploded view of the exemplary training device of FIG. 62, and additional aspects and embodiments thereof.

FIGS. 64A-64B depict a first housing of the exemplary training device of FIG. 62, according to aspects of the present disclosure.

FIGS. 65A-65B depict a second housing of the exemplary training device of FIG. 62, according to aspects of the present disclosure.

FIGS. 66A-66B depict a primary container of the exemplary training device of FIG. 62, according to aspects of the present disclosure.

FIGS. 67A-67D depict a rear cap of the exemplary training device of FIG. 62, according to aspects of the present disclosure.

FIGS. 68A-68B depict a plunger rod of the exemplary training device of FIG. 62, according to aspects of the present disclosure.

FIG. 69 depicts a piston of the exemplary safety device of FIG. 62, according to aspects of the present disclosure.

FIGS. 70A-70C depict a cap for of the exemplary training device of FIG. 62, according to aspects of the present disclosure.

FIGS. 71A-71G depict an exemplary method for using a training device, according to aspects of the present disclosure.

### DETAILED DESCRIPTION

Reference will now be made in detail to examples of the present disclosure, which are illustrated in the accompanying drawings. Wherever possible, the same reference numbers will be used throughout the drawings to refer to the same or like parts.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “or” is inclusive and is intended to mean that a process, method, article, or apparatus that comprises a list of elements may include a combination of or all of the elements. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Various safety concerns may arise with the use of a drug delivery device. Safety issues and concerns may occur prior to, during, and/or after use, e.g., injection of a product, e.g., a liquid medicament, from the drug delivery device. For example, premature expulsion of the product may occur if components of the device are not secure and/or locked into a proper pre-injection position. Exposure of the needle may cause accidental needle stick during injection if the device is accidentally removed from the injection site, and/or after injection when the device is removed from the injection site and disposed of. Exposure of the needle may also adversely affect users with needle-phobia and/or needle-related anxiety. In addition, there may be concerns of the user inserting and/or injecting the needle at an incorrect depth into the injection site.

To address such concerns, conventional safety devices have been developed. Conventional safety devices may include active safety mechanisms. Active safety mechanisms refer to mechanisms where the user is required to actively perform a specific action to trigger the safety mechanism. It may be difficult for lay users, e.g., users without medical training, to properly activate an active safety mechanism, while simultaneously properly injecting a medicament into themselves or another person. Further, active safety mechanisms may not have any effect if the user fails to initiate the safety mechanism. Passive safety mechanisms have been developed to address such issues. Devices including passive safety mechanisms do not require the user to perform any additional and/or separate action to trigger a safety mechanism, prior to, during, and/or after use of the device. However, other concerns may occur with the use of a passive drug delivery device. For example, if a user accidentally removes the device from the injection site, a passive safety mechanism may automatically activate prior to completion of the dose. In such case, the user may not be able to complete the dose and would have to obtain another device.

### Drug Delivery Safety Devices

Embodiments of the present disclosure relate to a drug delivery device, and in particular, a device for passively activating a safety mechanism during and/or after injection of a medicament from a product container, e.g., syringe

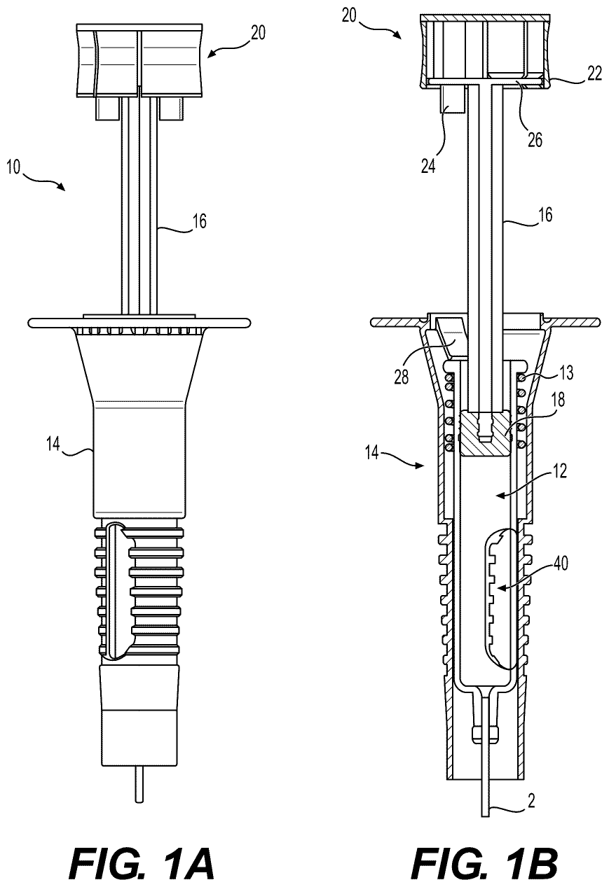

FIG. 1A shows an exterior view of a device 10. Device 10 may be designed to hold any commercially known product container 12 (e.g., a syringe or other suitable drug delivery device). Device 10 may include a housing 14, plunger rod 16, and thumb pad 20. FIG. 1B shows an interior view of device 10, wherein housing 14 may be configured to house container 12. In the embodiment shown in FIG. 1B, container 12 may be a syringe, e.g., a prefilled syringe. Housing 14 may include an inward projecting shoulder 28. As shown in FIG. 1B, shoulder 28 may be located in a proximal portion of housing 14 and may abut a proximal end of container 12 when housed in housing 14. Plunger rod 16 may couple to a stopper 18 for expelling product from container 12 and a flange 26 for coupling to thumb pad 20. Referring to FIG. 1B, stopper 18 may be positioned at the distal end of plunger rod 16, and flange 26 may be positioned at the proximal end of plunger rod 16.

As will be described in detail below, plunger rod 16 may abut and/or releasably couple to a portion of a syringe, e.g., a piston of the syringe, in housing 14. In some embodiments, plunger rod 16, thumb pad 20, and the syringe, may be locked together during injection, transmitting the user's force on plunger rod 16 to a piston of the syringe. After injection is complete, plunger rod 16 may decouple from the thumb pad 20. After plunger rod 16 decouples from thumb pad 20, thumb pad 20 may couple to housing 14, preventing thumb pad 20, plunger rod 16, and/or the syringe, from moving in a proximal direction beyond the thumb pad position (I.e. be removed from the housing). As thumb pad 20 moves in a distal direction to housing 14, a spring 13 may move the syringe and plunger rod 16 in a proximal direction into housing 14 and thumb pad 20, respectively.

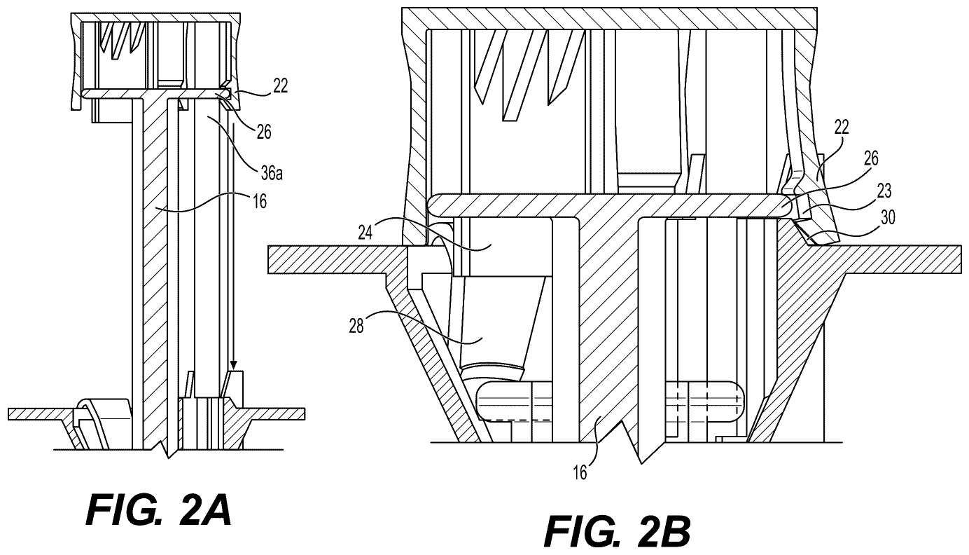

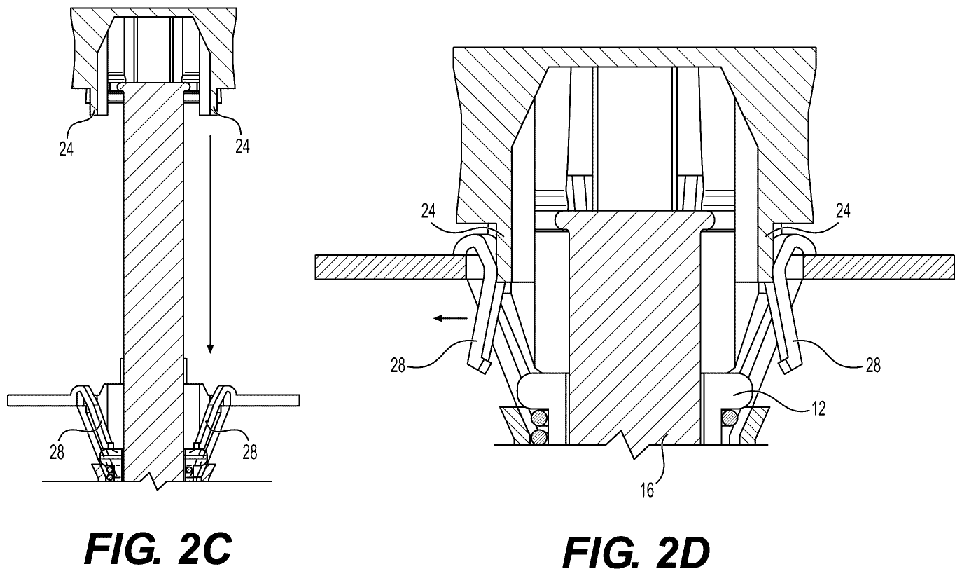

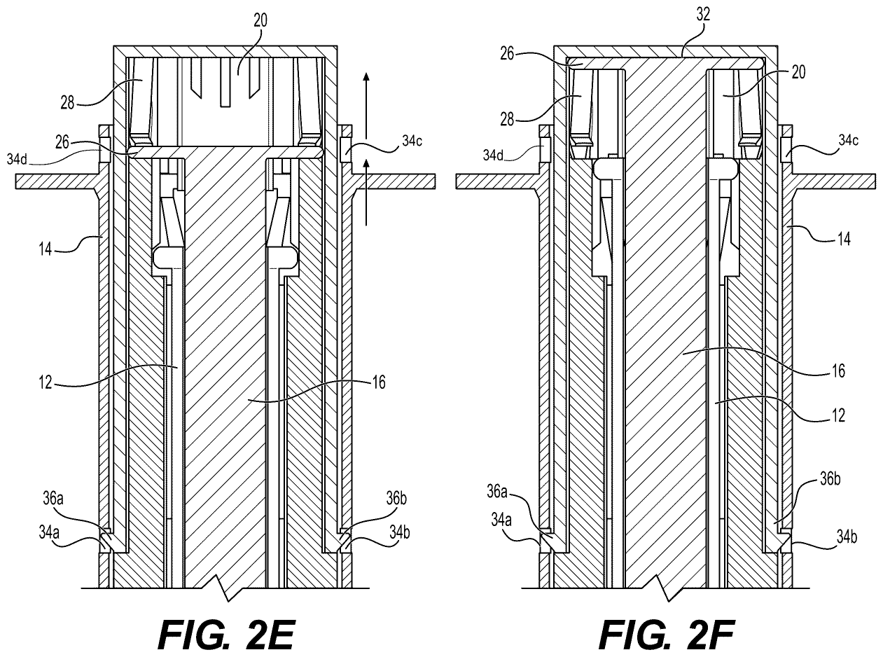

Various components may be utilized to move plunger rod 16 and/or syringe and then lock thumb pad 20, plunger rod 16, and/or the syringe into housing 14. In some embodiments, thumb pad 20 may include a deflectable tab 22 and distal extension 24. As shown in FIG. 2B, deflectable tab 22 may include a recess 23. Recess 23 may be releasably coupled to flange 26 of plunger rod 16. In some embodiments, deflectable tab 22 and recess 23 may be formed continuously or around a majority of an inner portion of thumb pad 20. In other words, deflectable tab 22 and recess 23 may be formed in thumb pad 20 to surround flange 26. In other embodiments, thumb pad 20 may include more than one deflectable tab 22 and recess 23. For example, thumb pad 20 may include two deflectable tabs, three deflectable tabs, or four deflectable tabs, each with a recess 23. In configurations with more than one deflectable tab 22 and recess 23, the tabs may be equally spaced apart from one another. For example, if the thumb pad 20 includes four deflectable tabs, each of the tabs may be about 90 degrees from one another. In addition, each deflectable tab may include a recess 23 for releasably coupling to a portion of flange 26.

In some embodiments, thumb pad 20 may include more than one distal extension 24. For example, thumb pad 20 may include two, three, four, or more distal extensions 24. In configurations with more than one distal extension 24, the distal extensions may be equally spaced apart from one another. For example, if thumb pad 20 includes four distal extensions 24, each of the distal extensions may be about 90 degrees from one another. In some embodiments, a single distal extension may extend continuously or at least partially around thumb pad 20.

Prior to injection, plunger rod 16 may be fully extended from a proximal end of housing 14 (FIG. 1B). In such a configuration, container 12 (e.g., a filled syringe) and plunger rod 16 may not be removed from device 10. Various components may prevent removal of plunger rod 16 from device 10. For example, arm 36a of thumb pad 20 may lock into cutouts 34c, 34d of housing 14 prior to injection and housing 14 to prevent the removal of plunger rod 16, as shown in FIG. 2E. Referring to FIGS. 2A-2B, to initiate injection of the product from container 12, a user may depress thumb pad 20 to move thumb pad 20 and plunger rod 16 in a distal direction towards housing 14. After the product is fully expelled from container 12 at the end of the injection dose, thumb pad 20 may abut the proximal end of housing 14 (FIG. 2B). The proximal end of housing 14 may include a collar 30 aligned with deflectable tab 22. As shown in FIG. 2B, collar 30 may have a beveled surface configured to abut and flex deflectable tab 22 in an outwards direction away from flange 26 as the injection dose is completed. Deflectable tab 22 may also include a beveled surface configured to abut the beveled surface of collar 30 to facilitate deflection of deflectable tab 22. Thumb pad 20, tab 22, and recess 23, may decouple from flange 26 and release plunger rod 16. FIG. 2A also depicts an arm 36a of thumb pad 20, which will be described in more detail below in reference to the lockout feature of device 10.

Also shown in FIG. 2B, once thumb pad 20 abuts housing 14, at the end of the injection dose, distal extension 24 of thumb pad may contact and deflect shoulder 28 of housing 14 in an outwards direction away from plunger rod 16. FIG. 2C depicts distal extensions 24 and shoulders 28 before they are moved into contact with one another, and FIG. 2D depicts distal extensions 24 of thumb pad 20 as they begin to contact shoulders 28. Once shoulder 28 is deflected in an outwards direction away from plunger rod 16 and the proximal end of container 12, container 12 may be released, allowing for movement of container 12 in a proximal direction. In some examples, a spring 13, e.g., a spring shroud, may abut a portion of container 12 in housing 14, in order to push container 12 in a proximal direction once container 12 is released from shoulder 28. For example, an at least partially compressed spring 13 may contact a distal surface of a flange of container 12, and when shoulder 28 is deflected outwards so as to no longer abut container 12, the spring may push the distal surface of the flange of container 12, and thus the entire container 12, in a proximal direction. The inclusion of a spring may provide a lockout function to device 10 to prevent re-use of the device. One spring may surround container 12 in housing 14, or one or more springs may be positioned adjacent to container 12 and may abut a distal surface of a portion of container 12.

FIG. 2E depicts device 10 when thumb pad 20 initially abuts the proximal end of housing 14, prior to lockout of device 10, and FIG. 2F depicts device 10 once locked out, with plunger rod 16 and flange 26 seated in thumb pad 20, such that flange 26 abuts a distal interior wall 32 of thumb pad 20. Once plunger rod 16 and flange 26 are released from recess 23 of deflectable tab 22, and the distal end of container 12 is released from shoulder 28, plunger rod 16, flange 26, and container 12, may move in a proximal direction towards a distal interior wall 32 of thumb pad 20 (FIG. 2F).

In some embodiments, thumb pad 20 may include one or more arms, e.g., arms 36a and 36b that extend distally into housing 14 as thumb pad 20 is depressed. A distal end of arms 36a, 36b may protrude radially outwards away from container 12, and arms 36a, 36b may be biased radially outwards. Housing 14 may include a plurality of cutouts 34a, 34b, 34c, 34d. Prior to injection, a distal end of arms 36a, 36b may align with cutouts 34c, 34d to maintain thumb pad 20 in place prior to a user applying a depressing force to plunger rod 16. When thumb pad 20 abuts the distal end of housing 14, releasing flange 26, plunger rod 16, and container 12, the protrusions on the distal ends of arms 36a, 36b may align with cutouts 34a, 34b and may deflect outwards to engage cutouts 34a, 34b (FIG. 2E). The engagement of the distal ends of arms 36a, 36b with cutouts 34a, 34b may maintain thumb pad 20 in place as flange 26 moves proximally to abut distal interior wall 32 of thumb pad 20 (FIG. 2F). It also serves to lock thumb pad 20 to housing 14, preventing re-use of device 10.

In some embodiments, multiple arms may extend distally from thumb pad 20. For example, thumb pad 20 may include two, three, four, or more arms 36a, 36b. In configurations with more than one arm, the arms may be equally spaced apart from one another. For example, if the thumb pad 20 includes four arms, each of the arms may be about 90 degrees from one another. If the thumb pad 20 includes two arms, the arms may be about 180 degrees from one another. Further, housing 14 may include a complimentary number of cutouts aligned with projections on the distal ends of the arms when thumb pad 20 is in a distal-most position. In some embodiments, a single arm may extend from thumb pad 20, and housing 14 may include a single cutout aligned with that arm. In some embodiments, thumb pad 20 may include two arms, and housing 14 may include two cutouts, one aligned with each arm, and so on. Although cutouts 34a, 34b are depicted as extending through the wall of housing 14, it is contemplated that cutouts 34a, 34b may be indentations or grooves that may not extend fully through housing 14. In embodiments in which the cutouts are grooves, one or more grooves may be positioned in line with the arms on thumb pad 20, or a single groove may extend around the inner wall of housing 14.

The embodiments of FIGS. 1A-2F depict a device with a passive safety mechanism that may activate when the user nears end-of-dose, e.g., when a substantial amount of the product is fully expelled from container 12. The passive safety mechanism of FIGS. 1A-2F may allow for container 12 and injection needle 2 (FIG. 1B) to move in a proximal direction into housing 14 at the end of the dose, so that injection needle 2 may be fully retracted in housing 14 to prevent accidental needle stick post-injection. Container 12 and injection needle 2 may not be moved proximally into housing 14 until injection is complete and thumb pad 20 is pressed against the proximal end of housing 14 to free flange 26 and container 12. As shown in FIGS. 1A-1B, only a portion of needle 2 may be seen by the user. This may prevent needle-phobia and needle-related anxiety as compared to conventional drug delivery devices with exposed needles. In addition, exposing only a portion of needle 2 may allow for usage of device 10 without any pre-injection steps, e.g., pinching of the skin to alter the injection depth to compensate for a needle length that is longer compared to the exposed needle 2 of device 10. Device 10 may be sized so that the starting position of container 12 within housing 14 is calibrated so that only a portion of the needle is exposed, and the length of the exposed portion of needle 2 represents the desired insertion depth of needle 2.

In some embodiments, device 10 may include a feedback mechanism. Device 10 may include a visual feedback mechanism, e.g., thumb pad 20 and housing 14 being locked together and needle 2 being retracted into housing 14, so needle 2 cannot be seen by user once device 10 is pulled away from the injection site. In some examples, device 10 may include an optional opening 40 (FIG. 1B) for viewing container 12 and movement of plunger rod 16 and stopper 18 in container 12. In some aspects, device 10 may include an audible feedback mechanism, e.g., an audible “clicking” sound once thumb pad 20 and housing 14 connect and/or once flange 26 abuts distal interior wall 32 of thumb pad 20. In some aspects, device 10 may include a tactile feedback, e.g., a snap or vibration as thumb pad 20 and housing 14 connect and/or once flange 26 abuts distal interior wall 32 of thumb pad 20.

Device 10 may be of any suitable size and shape to hold or partially hold a product container 12, and/or to support and hold plunger rod 16 and stopper 18 in product container 12. Thumb pad 20 may be of any suitable size and shape to abut and lock into device 10. Components of thumb pad 20, including deflectable tab 22 and recess 23, may be of any suitable size and shape to receive flange 26. Cutouts 34a, 34b, may be of any suitable size and shape to receive arms 36a, 36b.

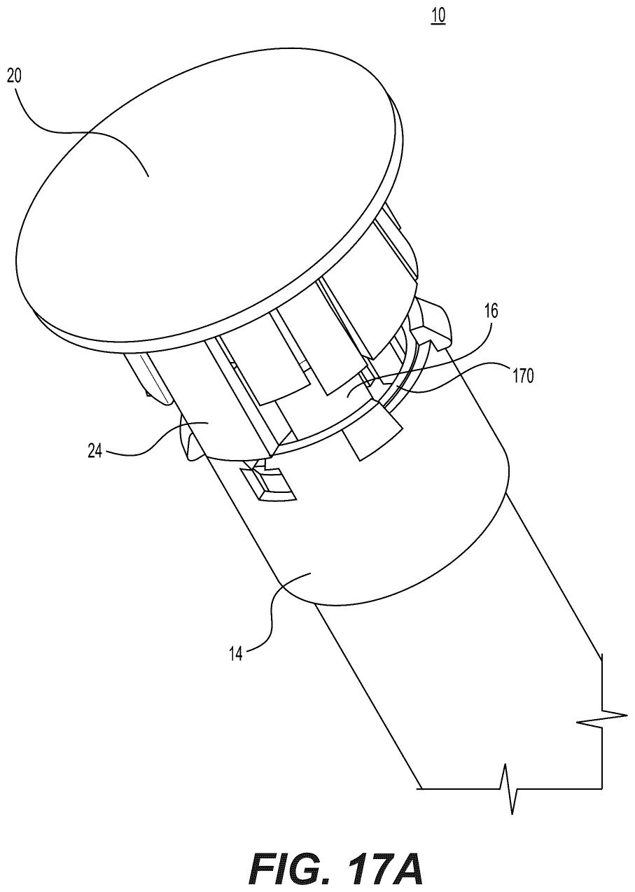

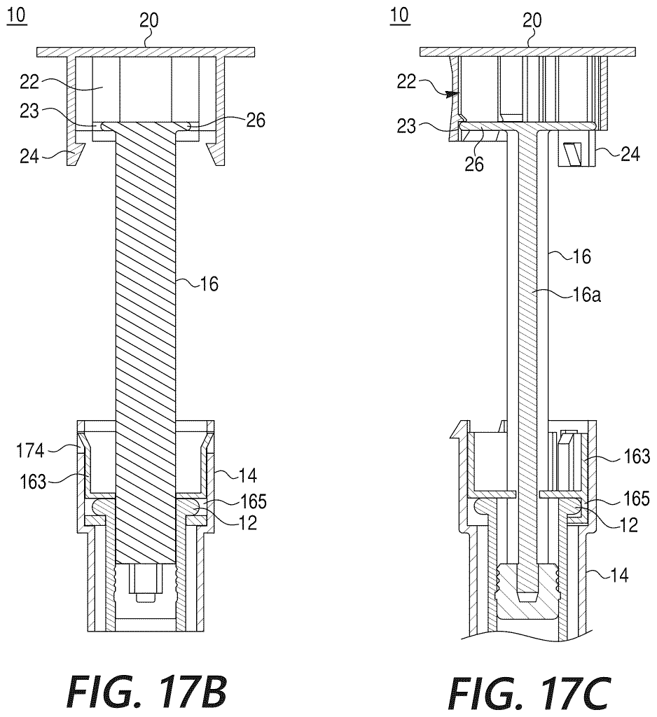

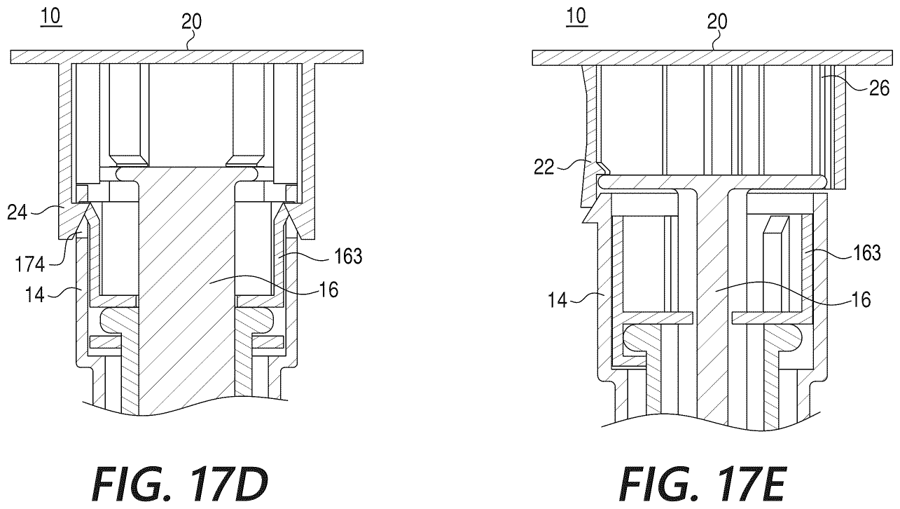

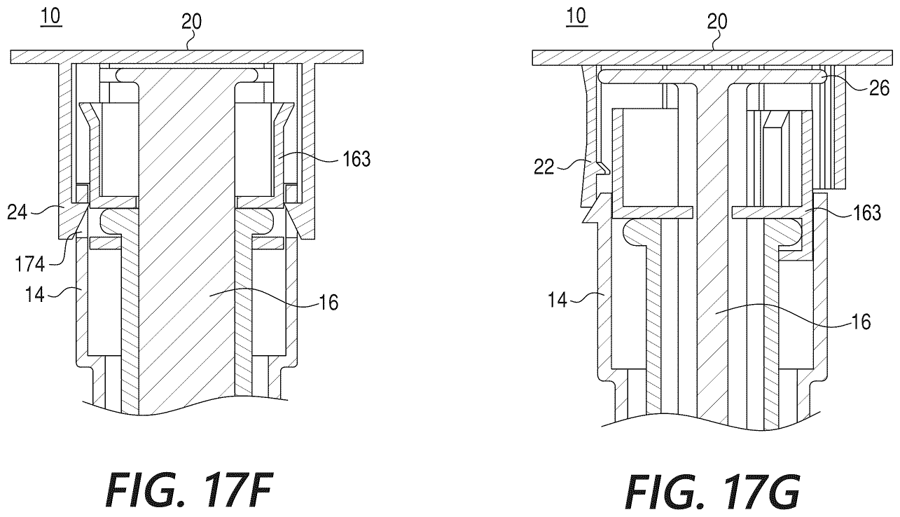

In other embodiments, device 10 may include a rear cap 163 (FIG. 17B). As shown in FIG. 17B, rear cap 163 may be shaped and configured to fit into a proximal end of housing 14. Rear cap 163 may have a substantially circular shape. A proximal end of rear cap 163 may abut a proximal end of housing 14 such that rear cap 163 may be secured in housing 14. Referring to FIG. 17B, rear cap 163 may include a rear cap recess 165 for receiving a portion of container 12. For example, a proximal portion of container 12 may fit into rear cap recess 165 of rear cap 163. Thumb pad 20 of device 10 may include deflectable tab 22. In some examples, thumb pad 20 may include a plurality of deflectable tabs 22. Deflectable tab 22 may include a recess 23 configured to receive a flange 26 of plunger rod 16. Thumb pad 20 may also include a distal extension 24. As shown in FIG. 17A, in some examples, thumb pad 20 may include a plurality of distal extensions 24. FIG. 17C depicts a rotated view of device 10 in FIG. 17B. Referring to FIG. 17C, plunger rod 16 may include a plurality of ridges 16a, wherein portions of rear cap 163 may abut against portions of ridges 16a.

During use, the user may press thumb pad 20 in a distal direction towards housing 14. Similar to the mechanism described in reference to FIGS. 2A and 2B, and referring to FIGS. 17D and 17E, as thumb pad 20 is pushed towards housing 14, a portion at the proximal end of housing 14 may abut deflectable tabs 22. Deflectable tabs 22 may deflect in a direction away from flange 26, allowing plunger rod 16 and thumb pad 20 to decouple. FIG. 17E depicts a rotated view of device 10 in FIG. 17D. Referring to FIG. 17F, at end-of-dose, a distal end of extensions 24 may click into housing 14. For example, housing 14 may include cutouts 174 for receiving the distal end of extensions 24. Once the distal end of extensions 24 click into cutouts 174, the distal end of extensions 24 may press a portion of rear cap 163 inwards towards plunger rod 16, allowing rear cap 163 to move into a proximal direction into thumb pad 20. Device 10 may include an actuator, e.g., a spring, (not shown) underneath rear cap 163. The actuator may be configured to push rear cap 163 into thumb pad 20 once a portion of rear cap 163 is depressed inwards towards plunger rod 16. FIG. 17G depicts a rotated view of device 10 in FIG. 17F.

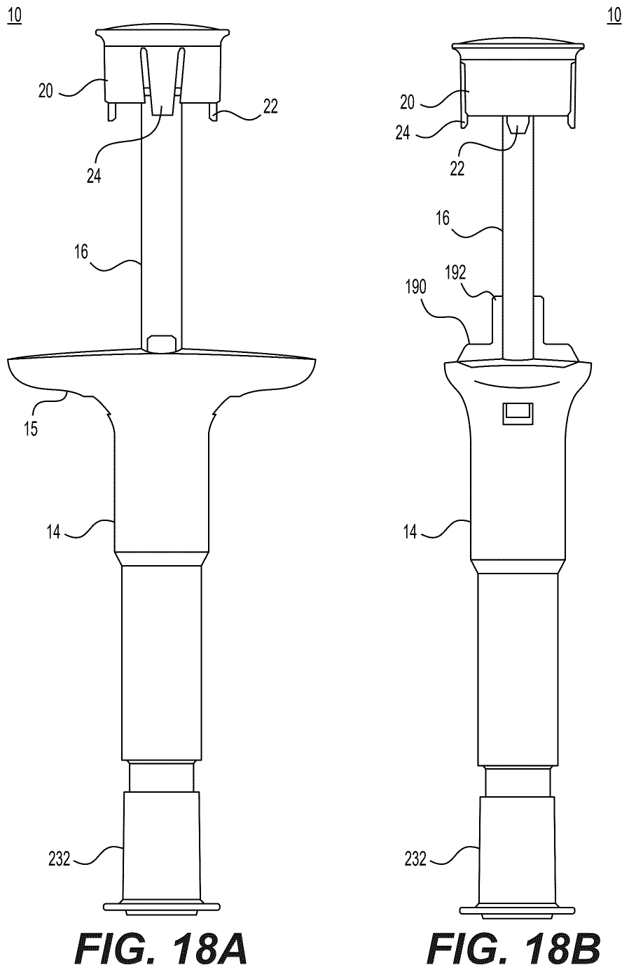

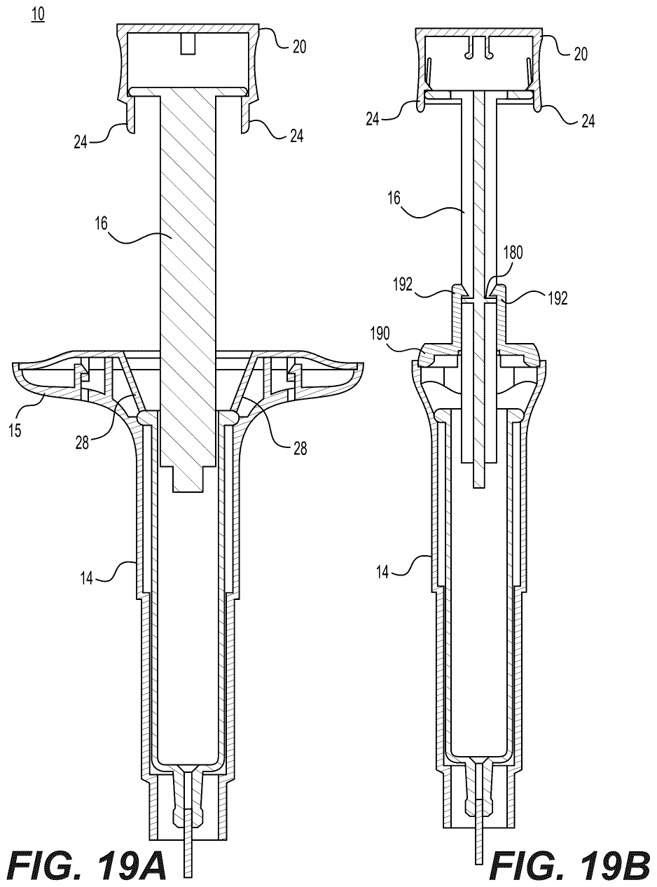

FIGS. 18A and 18B depict alternative embodiments of device 10. FIG. 18A depicts a frontal exterior view of device 10 and FIG. 18B depicts a side exterior view of device 10. Housing 14 of device 10 may include a finger flange 15. Finger flange 15 may be located at a proximal end of housing 14 and extend in a direction away from plunger rod 16. Finger flange 15 may be shaped or configured to be properly held by a user and properly operable with a syringe. For example, finger flange 15 may be designed to meet safety requirements and requisite needle distance requirements. Referring to FIG. 18B, housing 14 may include a rear cap 190. In this embodiment, rear cap 190 may be located at a proximal end of housing 14. Rear cap 190 may include a hook portion 192 extending in a proximal direction. As shown in FIG. 18B, rear cap 190 may include at least two hook portions 192. Hook portions 192 may sit against a recessed portion 180 of plunger rod 16 (shown in detail in FIGS. 19A and 19B). Hook portions 192 may hook onto, e.g., sit atop, a proximal surface of recessed portion 180.

FIGS. 19A and 19B depict cross-sectional views of device 10 in FIGS. 18A and 18B. FIG. 19A is a front cross-sectional view of device 10 and FIG. 19B is a side cross-sectional view of device 10. Thumb pad 20 may include distal extensions 24 and housing 14 may include finger flange 15. As discussed above, finger flange 15 may include rear cap 190, where rear cap 190 may include hook portions 192 that may hook onto recessed portions 180 of plunger rod 16. Referring to FIG. 19A, housing 14 may also include shoulder 28. Further details of housing 14 and rear cap 190 will be discussed below.

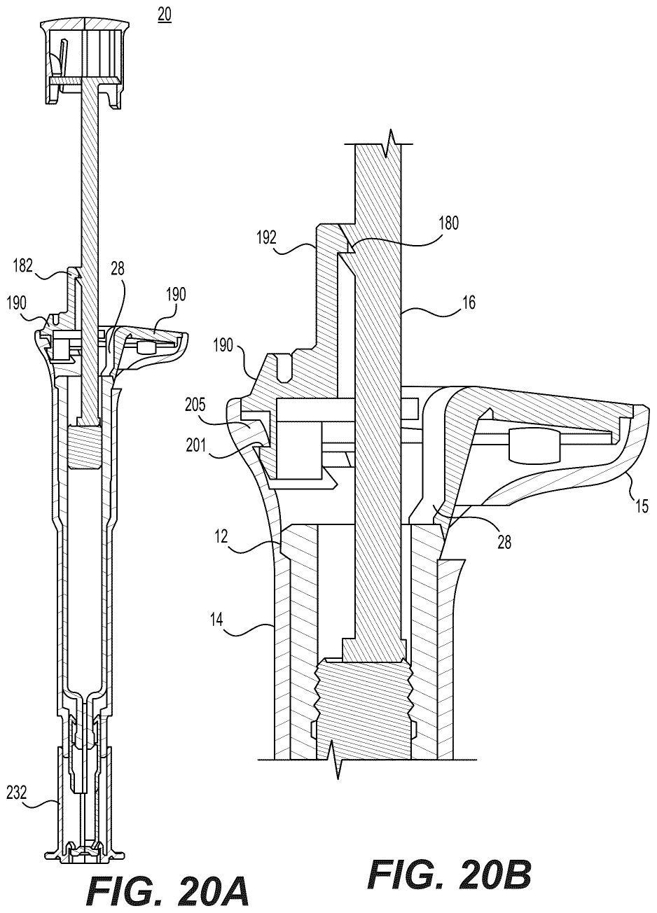

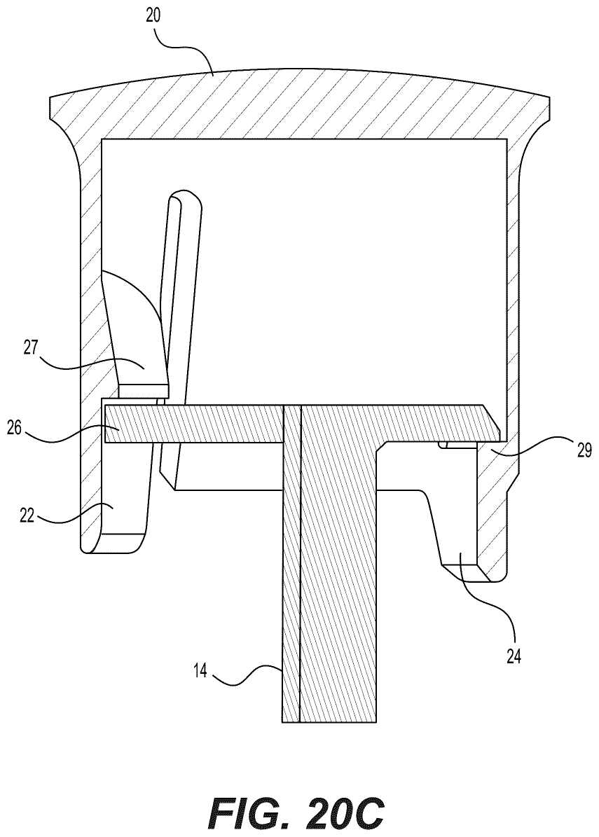

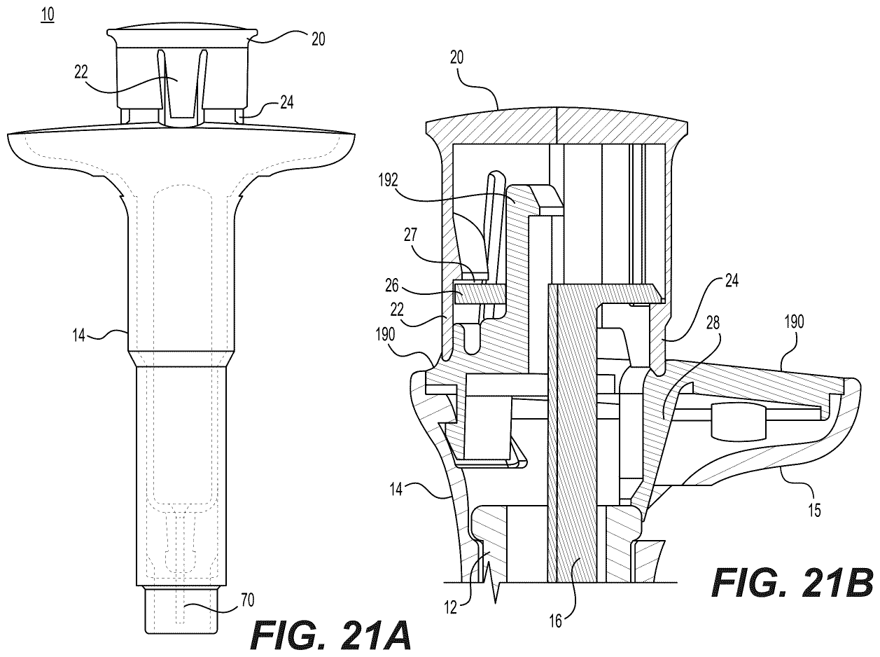

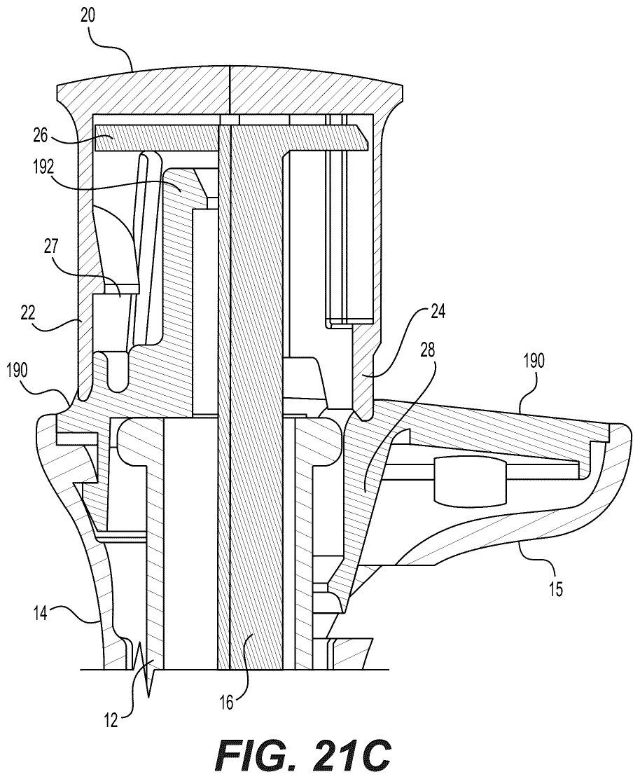

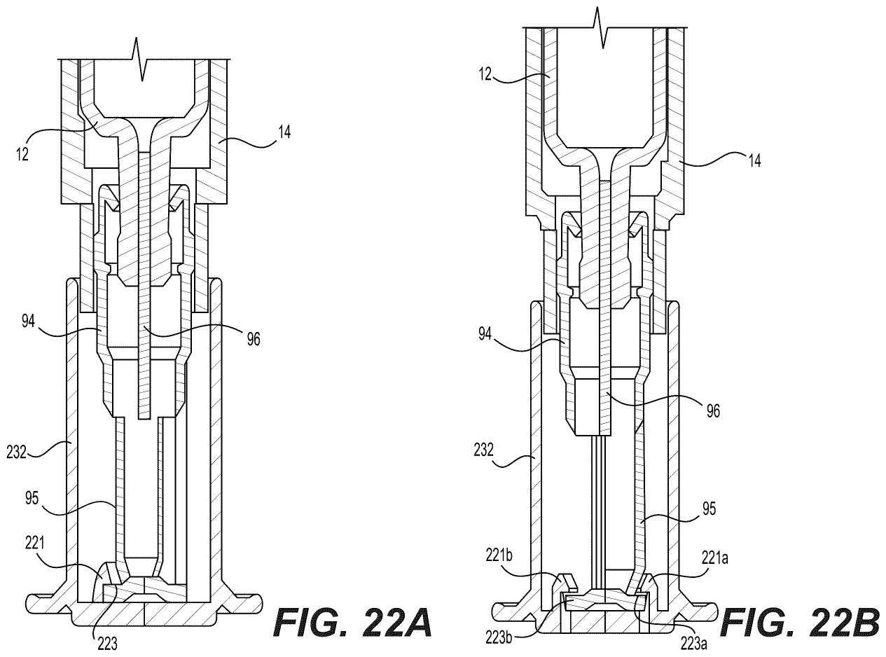

FIGS. 20A-20C depict views of device 10 in a pre-injection state. FIG. 20A depicts a quarter cross-sectional view of device 10, FIG. 20B depicts a detailed view of the proximal end of housing 14 and rear cap 190 from FIG. 20A, and FIG. 20C depicts a detailed view of thumb pad 20 from FIG. 20A. Referring to FIG. 20B, rear cap 190 and housing 14 may include attachment means for attaching rear cap 190 to housing 14. For example, rear cap 190 may include tabs 201 and housing 14 may include housing hooks 205. When rear cap 190 and housing 14 are attached (FIG. 20B), tabs 203 may sit into a portion of housing 14 and housing hooks 205 may hook onto, e.g., sit atop, a portion of tabs 201. This configuration may serve as a pull-back prevention feature preventing the user from inadvertently pulling plunger rod 16 out of the back of device 10. This configuration may also serve as a pre-expulsion prevention to prevent the loss of drug product before the needle is inserted into the skin. As shown in FIG. 20C, thumb pad 20 may include deflectable tab 22 and distal extension 24. FIG. 20C depicts a quarter cross-sectional view of thumb pad 20 from FIG. 20A. Thumb pad 20 may include a plurality of deflectable tabs 22 and distal extensions 24. Referring to FIG. 20C, a portion of flange 26 of plunger rod 16 abuts a distal surface of indent 27 of deflectable tab 22. Further, a portion of flange 26 may also sit atop a proximal surface of ledge 29 of distal extension 24. FIGS. 21A-21C depict device 10 in FIGS. 18A-20C as it transitions to a post-injection, e.g., end-of-dose state. At the start of injection, the needle of container 12 (not shown) may extend distally from housing 14. For example, about 6 mm of the needle may be exposed at the start of injection. While the user presses thumb pad 20 in a distal direction towards housing 14, plunger rod 16 may also extend distally into container 12, allowing for recessed portion 180 to detach from hook portion 192. Referring to FIG. 21B, once thumb pad 20 abuts rear cap 190, deflectable tab 22 may deflect outwards in a direction away from flange 26, allowing deflectable tab 22 to detach from flange 26. Once thumb pad 20 abuts rear cap 190, distal extension 24 may abut against shoulder 28, pushing shoulder 28 outwards in a direction away from container 12. Container 12 and plunger rod 16 may then move into a proximal direction, such that flange 26 of plunger rod 16 may abut a distal surface of thumb pad 20 (FIG. 21C). The needle 70 of container 12 may then be shrouded within housing 14 (FIG. 21A). This passive end-of-dose lockout mechanism may be activated by an actuator, e.g., spring 13, that may be located around a proximal end of container 12 (FIG. 1B).

Embodiments of device 10 depicted in FIGS. 18A-21C may include one or more additional features, e.g., features to lock thumb pad 20 in place with plunger rod 16 at a post-injection state; alignment features between thumb pad 20 and plunger rod 16 to reduce misalignment at the end of injection; ergonomic design to enable two hand operation, e.g., one hand may hold the syringe for needle insertion at 90 degrees, while the second hand pushes plunger rod 16; optimal configuration and sizing of deflectable tab 22, distal extension 24, plunger rod 16, and/or rear cap 190; indicators of injection completion, e.g., a visual color change or audible indicator; activation points to mitigate potential failure modes, e.g., a pre-activation point or state; optimal configuration and sizing of thumb pad 20 to fit a user's hand; extension of plunger rod 16 to sit within a piston of the device to improve alignment; and optimization of the design to reduce the required spring force to minimize plastic creep.

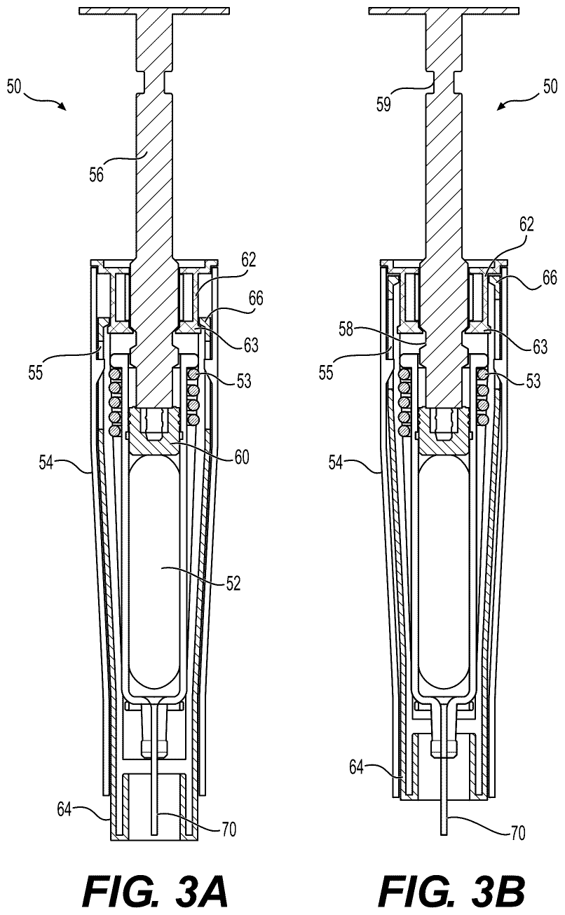

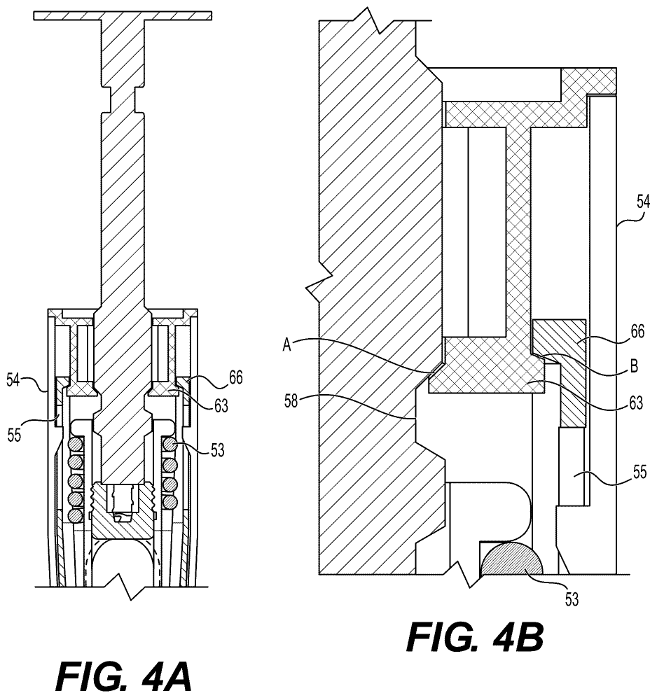

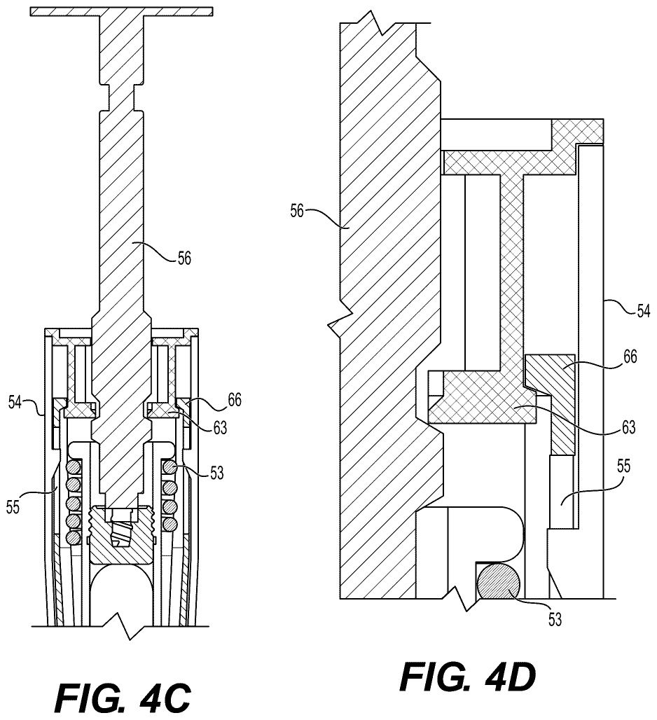

FIGS. 3A-5D depict another embodiment of the present disclosure in which the needle is covered in a pre-injection state. Referring to FIGS. 3A-3B, device 50 may be designed to hold any conventional product container 52, e.g., a syringe. Device 50 may include a housing 54, plunger rod 56, stopper 60, blocking component 62, and needle cover 64. As shown, for example, in FIG. 3B, plunger rod 56 may include a first indent 58 at a distal region and a second indent 59 at a proximal region. Blocking component 62 may be located at a proximal end of housing 54. Plunger rod 56 may be slidably coupled to blocking component 62, such that plunger rod 56 may slide in a distal direction through blocking component 62. Needle cover 64 may include a proximal end 66 abutting a deflectable arm 63 of blocking component 62. As shown in FIGS. 3A-3B, blocking component 62 may include more than one deflectable arm 63. For example, blocking component 62 may include two, three, four, or more deflectable arms 63. In configurations with more than one deflectable arm, the deflectable arms may be equally spaced apart from one another. For example, if the blocking component 62 includes four deflectable arms 63, each of the deflectable arms may be about 90 degrees from one another. For example, blocking component 62 may have a pair of deflectable arms 63 spaced, e.g., about 180 degrees from one another. Accordingly, the discussion herein may be applicable to a device with a varying number of components. In some examples, needle cover 64 may include a slot 55 (FIGS. 3A-4D) configured for receiving a portion of housing 54. Slot 55 will be further described below.

FIG. 3A depicts device 50 prior to use, where needle cover 64 may be fully extended distally of housing 54 and covers needle 70 in an initial, starting position. FIG. 3B depicts device 50 during use, once needle cover 64 may be depressed and moved proximally into housing 54, e.g., by pressing device 50 against the injection site. Detailed configurations and interactions of needle cover 64 and blocking component 62 will be further described as the use of device 50 is discussed.

At an initial position, prior to use, as shown in FIGS. 4A-4B, a distal end of deflectable arm 63 may fit into a portion of first indent 58 of plunger rod 56. For example, a distal portion of deflectable arm 63 may abut a portion of first indent 58 at contact point A. Distal end 66 of needle cover 64 may also abut a portion of deflectable arm 63 at a contact point B. At this initial position, prior to use, significant motion of plunger rod 56 in either a proximal or a distal direction may be prevented. For example, as shown in FIGS. 4A-4B, if plunger rod 56 is pushed in a distal direction into housing 54, deflectable arm 63 is prevented from deflecting outward by proximal end 66 of needle cover 64, locking deflectable arm 63 within first indent 58 and preventing further movement of plunger rod 56. As shown in FIGS. 4C-4D, if plunger rod 56 is pulled in a proximal direction away from housing 54, deflectable arm 63 is again prevented from deflecting outward by proximal end 66 of needle cover 64, locking deflectable arm 63 within first indent 58 and preventing further movement of plunger rod 56.

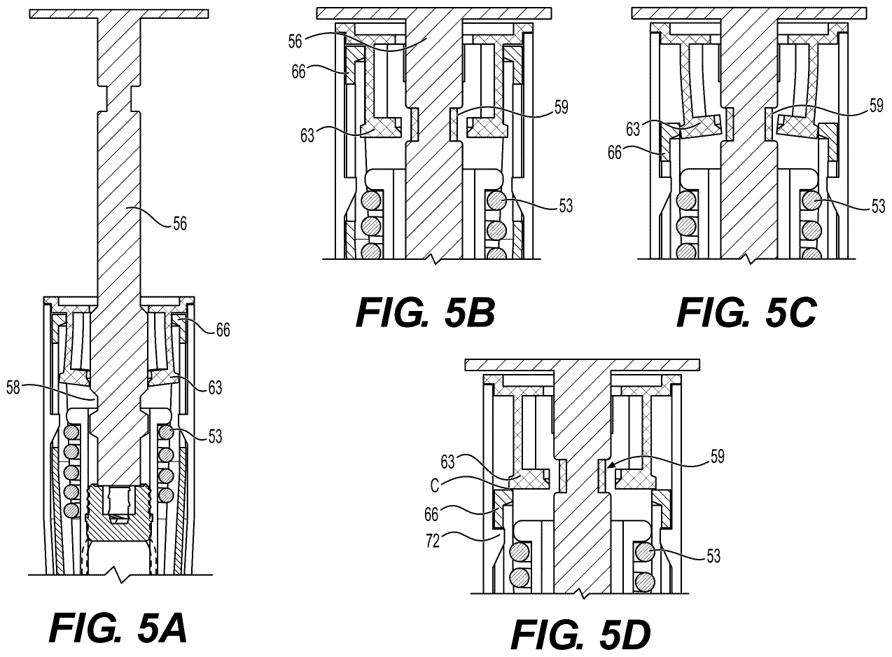

To initiate injection, device 50 may be pressed against an injection site to depress needle cover 64. Once needle cover 64 is moved distally, proximal end 66 of needle cover 64 moves proximally away from engagement with deflectable arm 63. This may allow plunger rod 56 to be pressed in a distal direction towards housing 54 for injection. In other words, as needle cover 64 moves proximally into housing 54 and proximal end 66 moves away from contact point B, plunger rod 56 may deflect arm 63 of blocking component 62, allowing first indent 58 to move past deflectable arm 63 (FIG. 5A) so that injection can proceed. As shown in FIG. 5A, proximal end 66 of needle cover 64 moves proximally in the housing and may abut a portion of blocking component 62.

Once injection is complete, device 50 may be removed from the injection site and needle cover 64 may be pulled downwards in a distal direction (FIGS. 5B-5C). In some examples, device 50 may include a spring shroud 53 (FIGS. 3A and 5A-5D) to pull needle cover 64 and bias needle cover 64 in a proximal direction. As show in FIGS. 5B-5C, plunger rod 56 is fully depressed into housing 54. As shown in FIGS. 4A and 4C, the portion of plunger rod 56 at second indent 59 may be smaller in diameter compared to the portion of plunger rod 56 at first indent 58, creating a larger second indent 59 compared to first indent 58. As such, once plunger rod 56 is fully depressed into housing 54 at the end of injection, there is an increased area between plunger rod 56 and deflectable arm 63 at second indent 59 (FIG. 5B). This space may allow for distal end 66 of needle cover 64 to flex deflectable arm 63 inwards towards plunger rod 56, so that proximal end 66 may move past deflectable arm 63 in a distal direction (FIG. 5C). In some examples, there may be a spring shroud 53 around needle cover 64, wherein needle cover 64 may move past deflectable arm 63 using spring force. Referring to FIG. 5D, once proximal end 66 has passed deflectable arm 63, deflectable arm 63 may return to an un-deflected position, and proximal end 66 may be stopped from moving in a proximal direction again by deflectable arm 63. Deflectable arm 63 may now act as a lockout mechanism to prevent re-use of device 50 or accidental needle stick, since needle cover 64 can no longer be moved proximally to expose the needle 70. At this final, post-injection position of device 50, needle cover 64 may be in a lockout position, and proximal end 66 and deflectable arm 63 may abut at contact point C (FIG. 5D). Needle cover 64 and proximal end 66 may be prevented from further movement, with proximal end 66 being held between ledge 72 of housing 54 and deflectable arm 63. Additionally, device 50 may not be re-used.

In some embodiments, slot 55 of needle cover 64 may be configured to slidably receive ledge 72. For example, as needle cover 64 moves, ledge 72 may move within slot 55. Ledge 72 may move in a distal and/or proximal direction within slot 55. As described above, at the post-injection position of device 50, proximal end 66 may be held between ledge 72 of housing 54 and deflectable arm 63, wherein ledge 72 may be within slot 55. As shown in FIG. 3A, needle cover 64 may include a complimentary number of slots aligned with ledges 72 of housing 54. In some embodiments, housing 54 may include a single ledge and needle cover 64 may include a single slot aligned with that ledge. In some embodiments, housing 54 may include two ledges and needle cover 64 may include two slots, one aligned with each ledge, and so on. Slot 55 may extend through needle cover 64. In other embodiments, slot 55 may be an indentation or groove that may not extend fully through needle cover 64.

The embodiments of FIGS. 3A-5D depict a device with a passive safety mechanism that may activate when the user nears end-of-dose, e.g., when a substantial amount of the product is fully expelled from container 52. The passive safety mechanism of FIGS. 3A-5D may prevent accidental post-injection needle stick, as needle cover 64 may not be depressed until device 50 is pressed against the injection site, and once device 50 is removed from the injection site, needle cover 64 springs back to extend from the distal end of housing 54 to cover needle 70.

Additionally, the passive safety mechanism of FIGS. 3A-5D may prevent premature lockout. For example, some conventional drug delivery devices may automatically lockout once the device is removed from the injection site, even if the product has not been fully expelled. Referring to FIGS. 5C-5D, only when plunger rod 56 is fully depressed into housing 54, can proximal end 66 of needle cover 64 flex deflectable arm 63 into second indent 59, so that proximal end 66 may move in a distal direction past deflectable arm 63 to the position adjacent ledge 72 to abut deflectable arm 63 at contact point C. Plunger rod 56 is only fully depressed at the end of the dose, and thus lockout may not occur prior to the entire dose of the product in container 52 having been expelled. If device 50 is removed from the injection during the injection, e.g., prior to plunger rod 56 being fully depressed into housing 54, needle cover 64 may not be locked out, and device 50 may be placed back onto the injection site to continue the injection.

Additionally, as needle 70 is within needle cover 64 before and after injection, the user may not see needle 70 at any point prior, during, and/or post injection. Needle cover 64 may thus reduce needle-phobia and needle-related anxiety as compared to conventional drug delivery devices with exposed needles. In addition, exposing only a portion of needle 70 may allow for usage of device 50 without any pre-injection steps, e.g., pinching the skin to alter the injection depth to compensation for a needle length that is longer compared to the exposed needle 70 of device 50 (FIG. 3B). Device 50 may be configured, e.g., container 52 may be positioned within housing 54 and/or needle cover 64 may be sized, so that the retracted position of needle cover 64 exposes only a portion of needle 70, and the length of the exposed portion of needle 70 represents the desired insertion depth of needle 70.

In some embodiments, device 50 may include a feedback mechanism. Device 50 may include a visual feedback mechanism, e.g., plunger rod 56 fully depressed into housing 54 (FIG. 5B) and needle cover 64 fully extended proximal to housing 54 with needle 70 covered. In some examples, device 50 may include an opening for viewing container 52 and movement of plunger rod 56 and stopper 60 in container 52. Device 50 may also include an audible feedback mechanism, e.g., an audible “clicking” sound once a proximal end of plunger rod 56 and housing 54 connect. In some aspects, device 50 may include a tactile feedback, e.g., a snap or vibration as needle cover 64 moves proximally and abuts ledge 72 at the end of the injection.

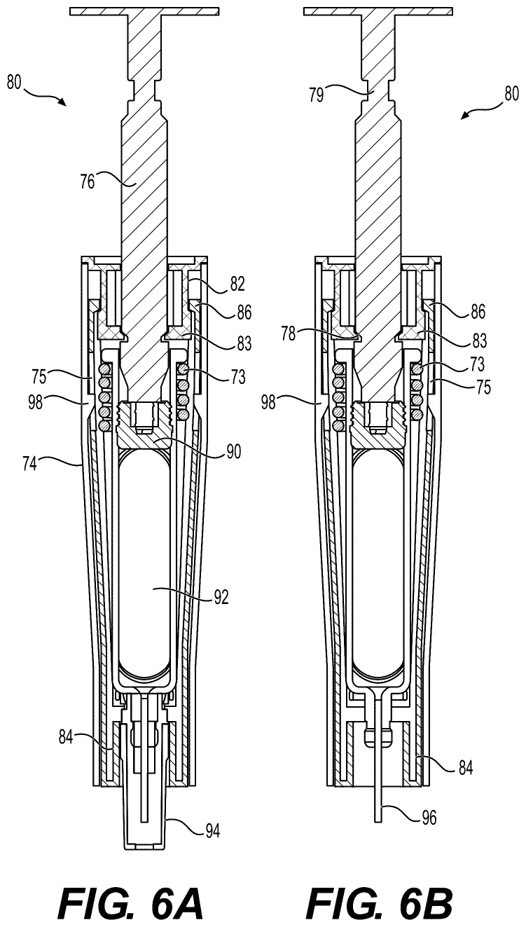

FIGS. 6A-6B depict another embodiment of the present disclosure, similar to those described above in reference to FIGS. 3A-5D, but in which the needle extends beyond the needle cover in a pre-injection state. Where appropriate, features will be designated with references similar to those discussed above. Any features and components that correspond to those in FIGS. 3A-5D may be understood to be configured similarly. Referring to FIGS. 6A-6B, device 80 may be designed to hold any conventional product container 92, e.g., a syringe. Device 80 may include a housing 74, plunger rod 76, stopper 90, blocking component 82, and needle cover 84. As shown, for example, in FIG. 6B, plunger rod 76 may include a first indent 78 at a distal region and a second indent 79 at a proximal region. Blocking component 82 may be located at a proximal end of housing 74. Plunger rod 76 may be slidably coupled to blocking component 82, such that plunger rod 76 may slide through blocking component 82. As shown in FIGS. 6A-6B, blocking component 82 may include more than one deflectable arm 83. For example, blocking component 82 may include two, three, four, or more deflectable arms 83. In configurations with more than one deflectable arm, the deflectable arms may be equally spaced apart from one another. For example, if the blocking component 82 includes four deflectable arms 63, each of the deflectable arms may be about 90 degrees from one another. For example, blocking component 82 may have a pair of deflectable arms 83 spaced, e.g., about 180 degrees from one another. Accordingly, the discussion herein may be applicable to a device with a varying number of components.

FIG. 6A depicts device 80 prior to use, where needle 96 extends distally from housing 74 and needle cover 84. An optional removable needle shield 94 may be inserted into or onto needle cover 84 and/or housing 74, to cover needle 96. FIG. 6B depicts device 80 prior to use, once needle shield 94 is removed. In contrast to the embodiments of FIGS. 3A-3B, since needle 96 extends beyond needle cover 84 in the device's initial state, the needle cover 84 may not need to be depressed prior to use. Once needle shield 94 is removed, needle 96 may be visible to a user prior to injection.

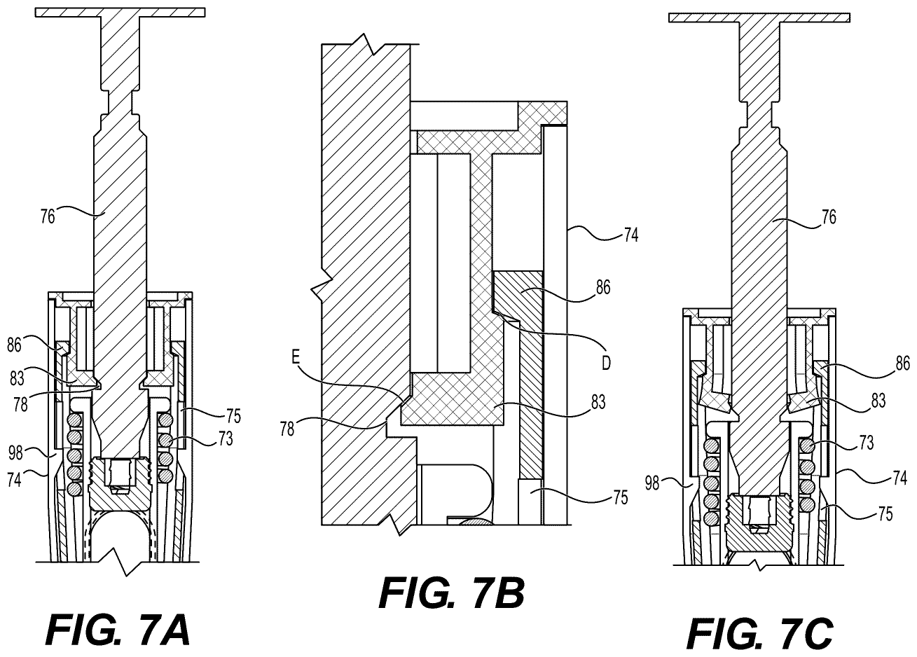

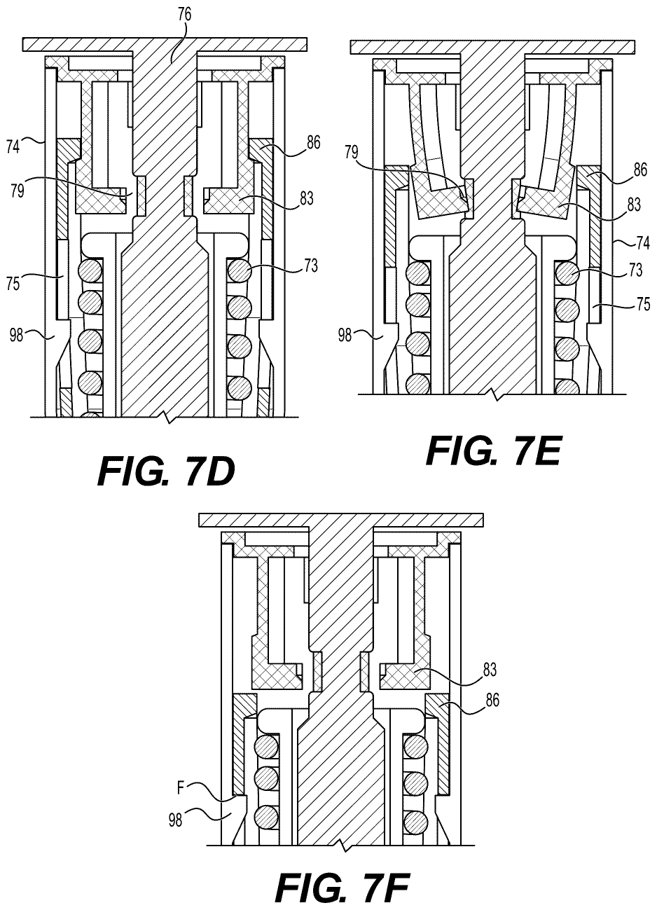

At an initial position, prior to use, as shown in FIGS. 7A-7B, a distal end of deflectable arm 83 may fit into at least a portion of first indent 78 of plunger rod 76. For example, a distal portion of deflectable arm 83 may abut a portion of first indent 78 at contact point E. Proximal end 86 of needle cover 84 may also abut a portion of deflectable arm 83 at a contact point D. At this initial position, prior to use, significant motion of plunger rod 76 in either a proximal or a distal direction may be prevented. For example, if plunger rod 76 is pushed in a distal direction into housing 74 using a break loose force, contact between deflectable arm 83 and plunger rod 76 at contact point E, as well as the contact between proximal end 86 of needle cover 84, may prevent proximal movement of plunger rod 76. The arrangement of deflectable arm 83, plunger rod 76 with first indent 78, and proximal end 86 of needle cover 84 may prevent movement of plunger rod 76 in a distal direction unless an increased amount of force is applied, e.g., a force higher than a typical break loose force. If plunger rod 76 is pulled in a proximal direction away from housing 74, deflectable arm 83 may contact an edge of first indent 78, preventing movement of plunger rod 76 in a distal direction.

To initiate injection, device 80 may be pressed against an injection site, allowing needle 96 to pierce injection site. Plunger rod 76 may then be pushed in a distal direction towards housing 74 using a force higher than normal break loose force. As plunger rod 76 is depressed into housing 74, deflectable arm 83 may be deflected radially outwards in a direction away from plunger rod 76 and contact point E when subjected to the higher than normal break loose force, allowing first indent 78 to move past deflectable arm 83 (FIG. 7C). A higher than normal break loose force may be, for example, a force above about 2 Newtons (N), e.g., about 3 N to about 20 N.

Once injection is complete, device 80 may be removed from the injection site, and as shown in FIG. 7D, plunger rod 76 may be fully depressed into housing 74, such that plunger rod 76 may abut housing 74. Once device 80 is removed from the injection site, needle cover 84 may include a spring shroud 73 (FIG. 6A) to move needle cover 84 downwards in a distal direction. As shown in FIGS. 6A-6B, the portion of plunger rod 76 at second indent 79 may be smaller in diameter compared to the portion of plunger rod 76 at first indent 78, creating a larger second indent 79 compared to first indent 78. As such, once plunger rod 76 is fully depressed into housing 74 at the end of injection, there is an increased area between plunger rod 76 and deflectable arm 83 at second indent 79 (FIG. 7D). This space may allow for proximal end 86 of needle cover 84 to flex deflectable arm 83 inwards towards plunger rod 76, so that proximal end 86 may move past deflectable arm 83 in a distal direction (FIG. 7E). In some examples, there may be a spring shroud (not shown) around needle cover 84 or at an end of needle cover 84, wherein needle cover 84 may move past deflectable arm 83 using spring force. Referring to FIG. 7E, once proximal end 86 has passed deflectable arm 83, deflectable arm 83 may flex back to its initial position, and needle cover 84 may be stopped from moving in a proximal direction again by deflectable arm 83. Deflectable arm 83 may now act as a lockout mechanism to prevent re-use of device 80 or accidental needle stick, since needle cover 84 may extend further distally compared to the starting position to that needle cover 84 covers needle 96. At this final, post-injection position of device 80, needle cover 84 may be extended distally in a lockout position, and needle cover 84 may abut ledge 98 at a contact point F, preventing further movement of needle cover 84 in a proximal direction. Proximal end 86 of needle cover 84 may also abut a distal end of deflectable arm 83 such that further proximal movement of needle cover 84 and proximal end 86 may be prevented.

In some examples, needle cover 84 may include a slot 75 (FIG. 6A) configured for receiving a portion of housing 74. In some embodiments, slot 75 of needle cover 84 may be configured to slidably receive ledge 98. For example, as needle cover 84 moves, ledge 98 may move within slot 75. Ledge 98 may move in a distal and/or proximal direction within slot 75. As described above, at the post-injection position of device 80, proximal end 86 may be held between ledge 98 of housing 74 and deflectable arm 83, wherein ledge 98 may be within slot 75. As shown in FIG. 6A, needle cover 84 may include a complimentary number of slots aligned with ledges 98 of housing 74. In some embodiments, housing 74 may include a single ledge and needle cover 84 may include a single slot aligned with that ledge. In some embodiments, housing 74 may include two ledges and needle cover 84 may include two slots, one aligned with each ledge, and so on. Slot 75 may extend through needle cover 84. In other embodiments, slot 75 may be an indentation or groove that may not extend fully through needle cover 84.

The embodiments of FIGS. 6A-7F depict a device with a passive safety mechanism that may activate when the user nears end-of-dose, e.g., when a substantial amount of the product is fully expelled from container 92. The passive safety mechanism of FIGS. 6A-7F may also prevent premature lockout. For example, some conventional drug delivery devices may automatically lock out once the device is removed from the injection site, even if the product has not been fully expelled. Referring to FIGS. 7D-7F, only when plunger rod 76 is fully depressed into housing 74, can proximal end 86 of needle cover 84 flex deflectable arm 83 into second indent 79, so that needle cover 84 may move in a distal direction past deflectable arm 83 and then abut ledge 98 at contact point F. Once plunger rod 76 is fully depressed, the entire dose of the product in container 92 may be expelled. If device 80 may be removed from the injection site during the injection, e.g., prior to plunger rod 76 being fully depressed into housing 74, needle cover 84 may not be locked out, and device 80 may be placed back onto the injection site to continue the injection.

Additionally, as a portion of needle 96 is within needle cover 84, the user may only see a portion of needle 96 at any point prior to and/or during injection. As needle cover 84 may extend from a proximal end of housing 74 to cover needle 96 post-injection, the user may not see any portion of needle 96 at end-of-dose. Needle cover 84 may thus reduce needle-phobia and needle-related anxiety as compared to conventional drug delivery devices with exposed needles. In addition, exposing only a portion of needle 96 may allow for usage of device 80 without any pre-injection steps, e.g., pinching the skin to alter the injection depth to compensation for a needle length that is longer compared to the exposed needle 96 of device 80 (FIG. 6B). Device 80 may be configured, e.g., container 92 may be positioned within housing 74 and/or needle cover 84 may be sized, so that needle cover 84 exposes only a portion of needle 96, and the length of the exposed portion of needle 96 represents the desired insertion depth of needle 96.

Referring to FIG. 7C, the safety mechanism may also prevent pre-expulsion of the product. As described above, the force exerted by deflectable arm 83 and proximal end 86 of needle cover 84 on plunger rod 76 prevents movement of plunger rod 76 until an initial “break” force, e.g., a greater than normal injection force, is produced to overcome the force between deflectable arm 83, proximal end 86, and plunger rod 76. The “break” force can be tuned as desired for the specific drug product, wherein the “break” force may be high enough force to prevent expelling the drug product too quickly, but low enough so that it may not be difficult for the user to initiate the injection.

In some embodiments, device 80 may include a feedback mechanism. Device 80 may include a visual feedback mechanism, e.g., plunger rod 76 fully depressed into housing 74 (FIG. 7B) and needle cover 84 fully extended distally to housing 74 with needle 96 covered. In some examples, device 80 may include an opening for viewing container 92 and movement of plunger rod 76 and stopper 90 in container 92. Device 80 may also include an audible feedback mechanism, e.g., an audible “clicking” sound once a proximal end of plunger rod 76 abuts housing 74, or once needle cover 84 is pushed distally from housing 74. In some aspects, device 80 may include a tactile feedback, e.g., a snap or vibration as needle cover 84 moves distally and abuts ledge 98 at the end of the injection.

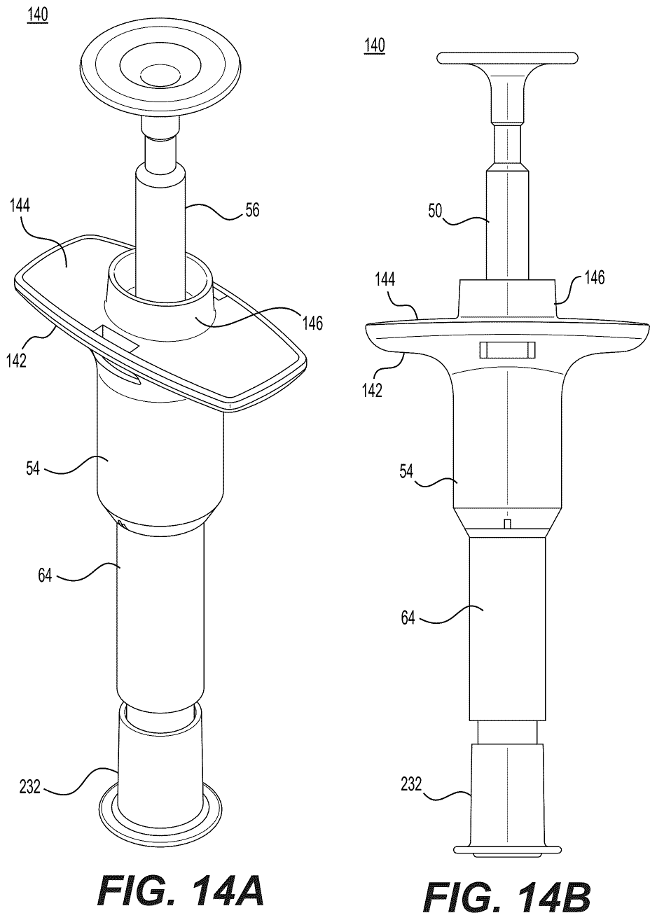

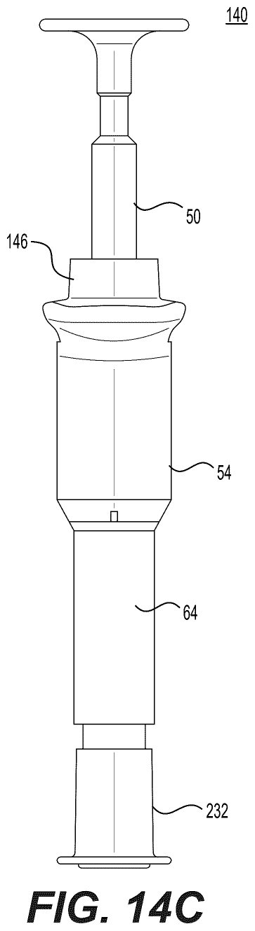

FIGS. 14A-14C depict another embodiment of the present disclosure, similar to those described above in reference to FIGS. 3A-7F. Where appropriate, features will be designated with references similar to those discussed above. Any features and components that correspond to those in FIGS. 3A-7F may be understood to be configured similarly. FIG. 14A shows a perspective view of the exterior of a device 140, FIG. 14B shows an exterior frontal view of device 140, and FIG. 14C shows an exterior side view of device 140. Device 140 may include plunger rod 56, housing 54, and needle cap 232. Housing 54 may include a finger flange 142 and rear cap 144. Finger flange 142 may have any appropriate size and/or configuration allowing for proper use. For example, a user may utilize two hands to operate device 140. One hand of the user may hold device 140, while the other hand of the user pushes plunger rod 56 in a distal direction towards finger flange 142. As shown in FIGS. 14A-14C, rear cap 144 may include a collar 146. Collar 146 may extend in a proximal direction away from finger flange 142. Collar 146 may be any appropriate size and/or configuration to surround a portion of plunger rod 56. In some embodiments, collar 146 may have a generally circular shape.

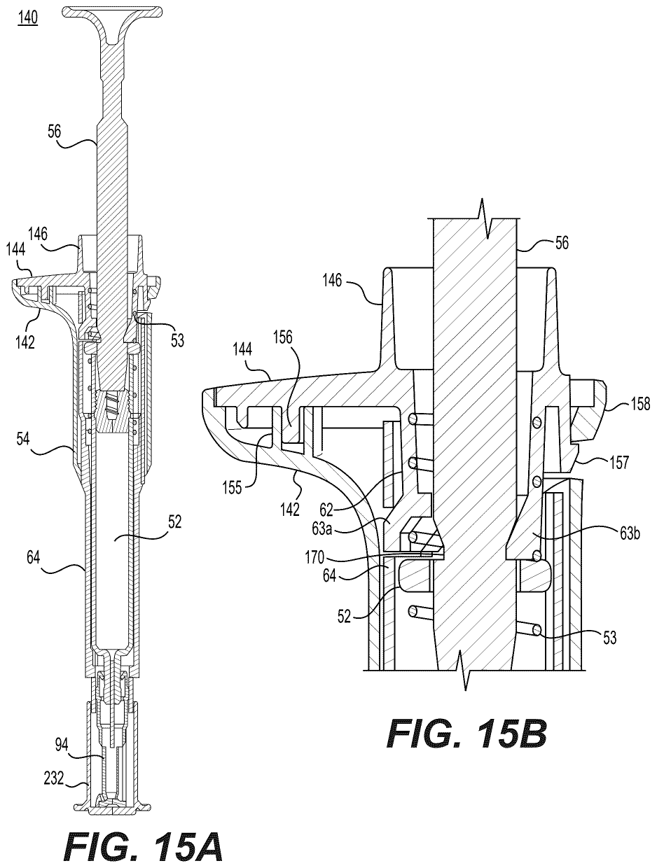

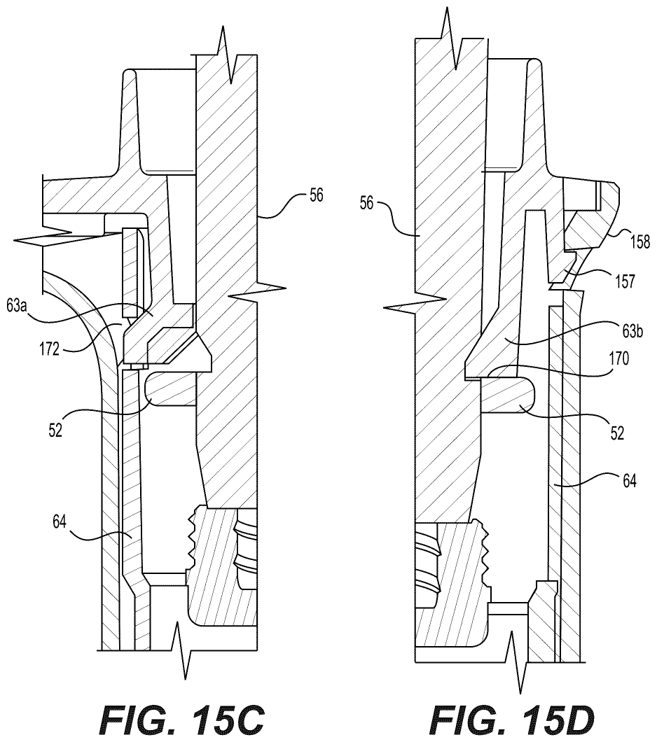

FIGS. 15A-15D depict a cross-sectional view of an interior of device 140. FIGS. 15A and 15B depict a cross-sectional view of a quarter portion of device 140. Referring to FIG. 15A, device 140 may include needle cover 64, syringe 52, spring shroud 53, needle cap 232, and needle shield 94. The configuration of needle cap 232 and needle shield 94 will be discussed in detail herein, e.g., in regards to FIGS. 22A and 22B. Referring to FIG. 15B, device 50 may include various features to couple finger flange 142 and rear cap 144 together. For example, finger flange 142 may include a snap 155 and rear cap 144 may include an insert 156. Snap 155 and insert 156 may engage, e.g., snap together, to couple rear cap 144 to finger flange 142. Rear cap 144 may also include a rear cap hook 149 for abutting against a ledge portion 158 of housing 54.

Referring to FIGS. 15B-15D, device 50 is shown in a pre-injection state. Rear cap 144 may include a blocking component 62. Plunger rod 56 may be include an indent 170 and may be slidably coupled to blocking component 62, such that plunger rod 56 may slide in both a distal and proximal direction. Blocking component 62 may include deflectable arm 63 extending in a distal direction. As shown in FIG. 15B, blocking component 62 may include more than one deflectable arm 63. For example, blocking component 62 may include two, three, four, or more deflectable arms 63. In configurations with more than one deflectable arm 63, the deflectable arms 63 may be equally spaced apart from one another. For example, if the blocking component 62 includes four deflectable arms 63, each of the deflectable arms 63 may be about 90 degrees from one another.

In a pre-injection state, one or more of deflectable arms 63 may abut against a portion of plunger rod 56. For example, one or more of deflectable arms 63 may abut against indent 170 of plunger rod 56 (FIG. 15B). Needle cover 64 may include an opening 172 for receiving a portion of one or more of deflectable arms 63. In a pre-injection state, a first deflectable arm 63a may abut against plunger rod 56, such that a portion of first deflectable arm 63a may fit into opening 172 (FIG. 15C). A second deflectable arm 63 may abut against indent 170 (FIG. 15D). At this initial position, prior to use, significant motion of plunger rod 56 in either a proximal or a distal direction may be prevented. For example, if plunger rod 56 is pushed in a distal direction into housing 54, first deflectable arm 63a is prevented from deflecting outward by a distal end of needle cover 64, locking deflectable arm 63 within opening 172 and preventing further movement of plunger rod 56. If plunger rod 56 is pulled in a proximal direction away from housing 54, second deflectable arm 63 b may be prevented from deflecting outward as second deflectable arm 63b abuts indent 170.

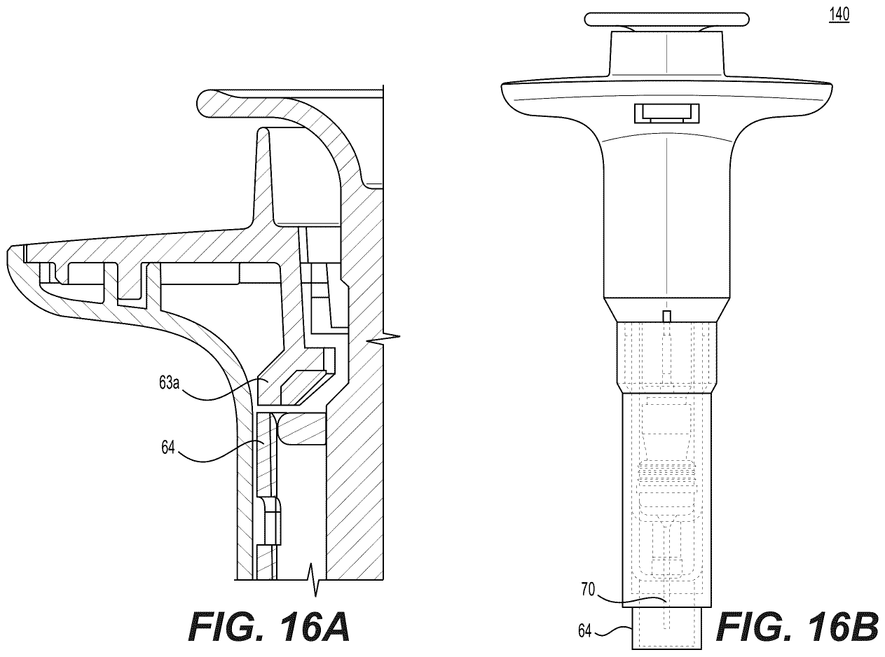

To initiate injection, device 140 may be pressed against an injection site to depress needle cover 64. As plunger rod 56 is pressed in a distal direction into housing 54, plunger rod 56 may deflect second deflectable arm 63b of blocking component 62, allowing plunger rod 56 to move distally into syringe 52 so that injection may proceed. Once injection is complete, device 140 may be removed from the injection site and needle cover 64 may automatically be pulled in a distal direction such that it cover the needle 70 (FIG. 16B). In some examples, spring shroud 53 may pull needle cover 64 into a distal direction. During injection, as plunger rod 56 moves distally into housing 54, first deflectable arm 63a may deflect inwards towards plunger rod 56, allowing first deflectable arm 63a to detach from opening 172. Referring to FIG. 16A, at the end of injection, and once first deflectable arm 63a is located proximally to needle cover 64, first deflectable arm 63a may return to an un-deflect position and needle cover 64 may be prevented from moving in a proximal direction. First deflectable arm 63a may now act as a lockout mechanism to prevent re-use of device 140 or accidental needle stick, since needle cover 64 may no longer be moved distally to expose needle 70. At this final, post-injection position of device 140, needle cover 64 may be in a lockout position and a proximal end of needle cover 64 and first deflectable arm 63a may abut one another. At this stage, device 140 may not be re-used.

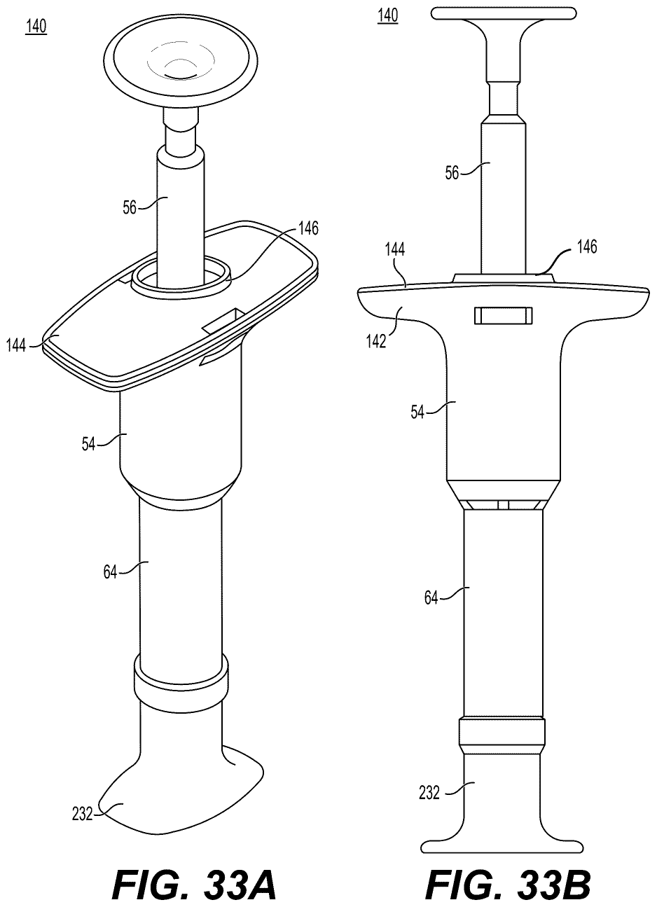

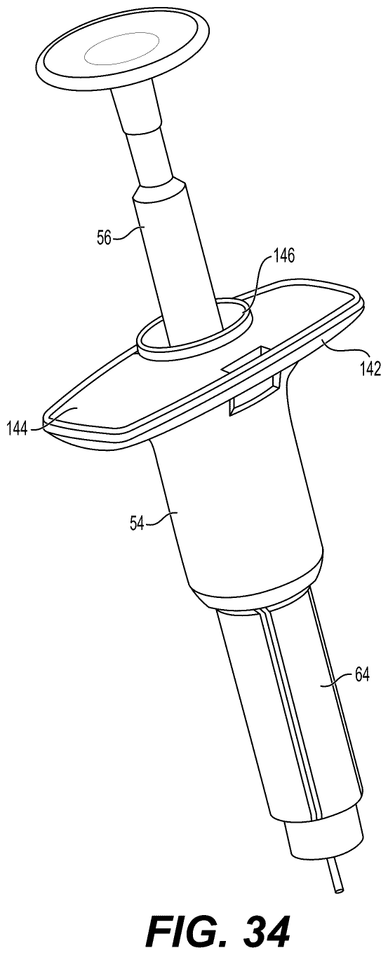

FIGS. 33A-33B depict another embodiment of the present disclosure, similar to those described above in reference to FIGS. 14A-16B. Where appropriate, features will be designated with references similar to those discussed above. Any features and components that correspond to those in FIGS. 14A-16B may be understood to be configured similarly. FIG. 33A shows a perspective view of the exterior of device 140, FIG. 33B shows an exterior frontal view of device 140, and FIG. 34 shows a perspective view of the exterior of device 140 without a needle cap 232. Device 140 may include plunger rod 56, housing 54, and needle cap 232. Housing 54 may include a finger flange 142 and rear cap 144. Rear cap 144 may also be referred to here as a blocking component. Finger flange 142 may have any appropriate size and/or configuration allowing for proper use. A user may utilize one or two hands to operate device 140. For example, one hand of the user may hold device 140, while the other hand of the user pushes plunger rod 56 in a distal direction towards finger flange 142. In other examples, the user may utilize one hand to both hold device 140 and push plunger rod 56 in a distal direction towards finger flange 142. As shown in FIGS. 33A-33B, rear cap 144 may include a collar 146. Collar 146 may extend in a proximal direction away from finger flange 142. Collar 146 may be any appropriate size and/or configuration to surround a portion of plunger rod 56. In some embodiments, collar 146 may have a generally circular shape.

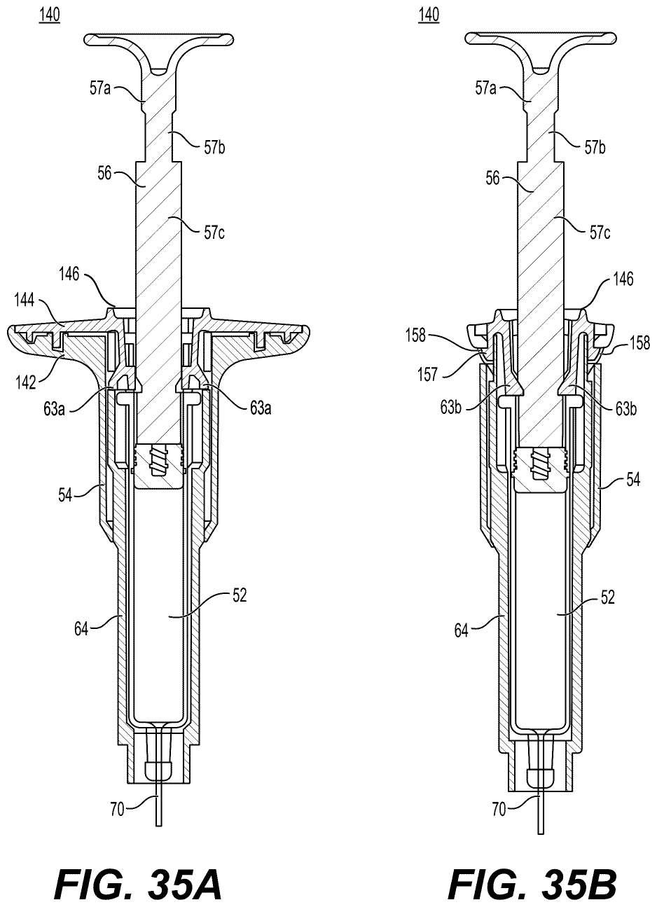

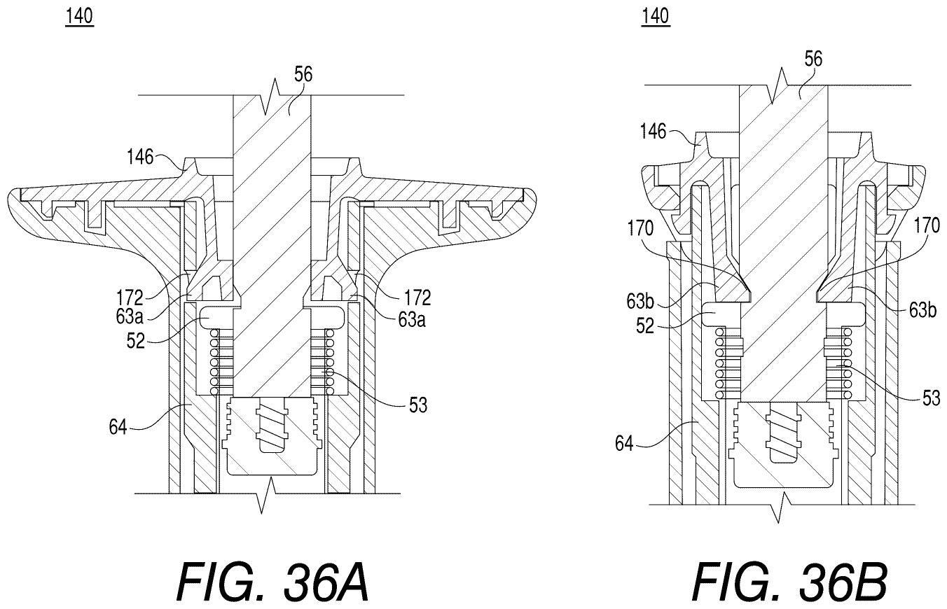

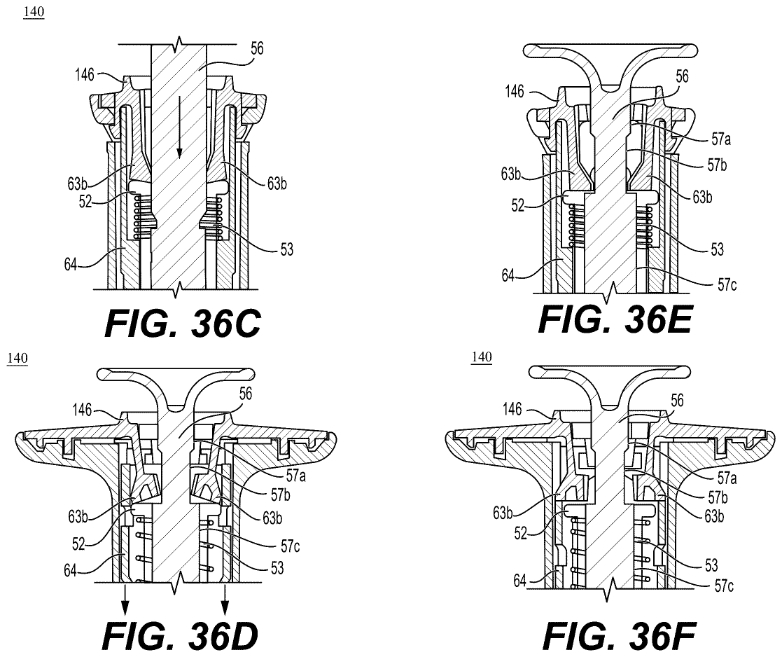

FIG. 35A depicts a front cross-sectional view of an interior of device 140 and FIG. 35B depicts a side cross-sectional view of an interior of device 140. Referring to FIG. 35A, device 140 may include plunger rod 56, housing 54, needle cover 64, and syringe 52. Still referring to FIG. 35A, device 140 may include various features to couple finger flange 142 and rear cap 144 together. Rear cap 144 may also include a plurality of deflectable arms 63a-63b. Referring to FIGS. 35A-35B, rear cap 144 may include a first plurality of deflectable arms 63a and a second plurality of deflectable arms 63b. Such features will be discussed in detail below in regards to FIGS. 36A-38B. As shown in FIGS. 35A-35B, rear cap 144 may include a rear cap hook 149 for abutting against a ledge portion 158 of housing 54. In some examples, rear cap 144 may include a plurality of rear cap hooks 149 for abutting against a plurality of ledge portions 158 of housing 54. For example, rear cap 144 may include at least two rear cap hooks 149, at least three rear cap hooks 149, or at least four rear cap hooks 149. As shown in FIG. 35B, housing 54 may include at least one ledge portion 158, at least two ledge portions 158, at least three ledge portions 158, or at least four ledge portions 158. In some examples, plunger rod 56 may include a first portion 57a, a second portion 57b, and a third portion 57c. First portion 57a may have a first diameter, second portion 57b may have a second diameter, and third portion 57c may have a third diameter. In some examples, the first diameter and the third diameter may each be larger than the second diameter. In some embodiments, third diameter of third portion 57c may be larger than first diameter of first portion 57a. Second portion 57b may also be referred to herein as an indent portion.

FIGS. 36A-36F depict device 140 in various states of use. Referring to FIGS. 36A-36B, device 140 is shown in a pre-injection state, e.g., a “ready-to-dose” stage. Spring shroud 53 may be in a compressed stage, where spring shroud 53 pushes needle cover 64 in a distal direction, as designated in FIGS. 36C and 36D by the downwards pointing arrow. In some examples, plunger rod 56 may include at least one indent 170. Referring to FIG. 36B, indent 170 may have any appropriate shape and/or configuration for receiving at least one arm of the second pair of deflectable arms 63b. For example, each indent 170 may have a wall that inclines radially inward in the distal direction and a proximally facing wall. In some embodiments, plunger rod 56 may have single indent 170 that extends around the circumference of plunger rod 56. In some examples, each arm of the second pair of deflectable arms 63b may abut against a corresponding indent 170 of plunger rod 56. Still referring to FIG. 36B, each arm of the second pair of deflectable arms 63b may include a radially inward extending incline that may correspond to indent 170.

In some examples, needle cover 64 may include at least one opening 172 for receiving a portion of one or more of the first pair of deflectable arms 63a. Opening 172 may extend through an outer sidewall of needle cover 64. As shown in FIG. 36A, opening 172 may be located at a proximal portion of needle cover 64. In some examples, a portion of at least one deflectable arm of the first pair of deflectable arms 63a may fit into opening 172 (FIG. 36A). In other examples, a portion of each arm of the first pair of deflectable arms 63a may be received into a corresponding opening 172. As shown in FIG. 36B, at least one deflectable arm of the second pair of deflectable arms 63b may abut against a portion of plunger rod 56. At this pre-injection state, prior to use, significant motion of plunger rod 56 in either a proximal or a distal direction may be prevented or otherwise inhibited, and as a result prevents accidental initiation of the dosage and/or expulsion of the medicament. For example, if plunger rod 56 is pushed in a distal direction into housing 54 with a force below a typical break loose force, second pair of deflectable arms 63b may not bend, thus preventing further movement of plunger rod 56 (FIG. 36A). If plunger rod 56 is pulled in a proximal direction away from housing 54, second pair of deflectable arms 63 b may be prevented from deflecting outward as each of the second pair of deflectable arms 63b abuts their corresponding indent 170 (FIG. 36B).

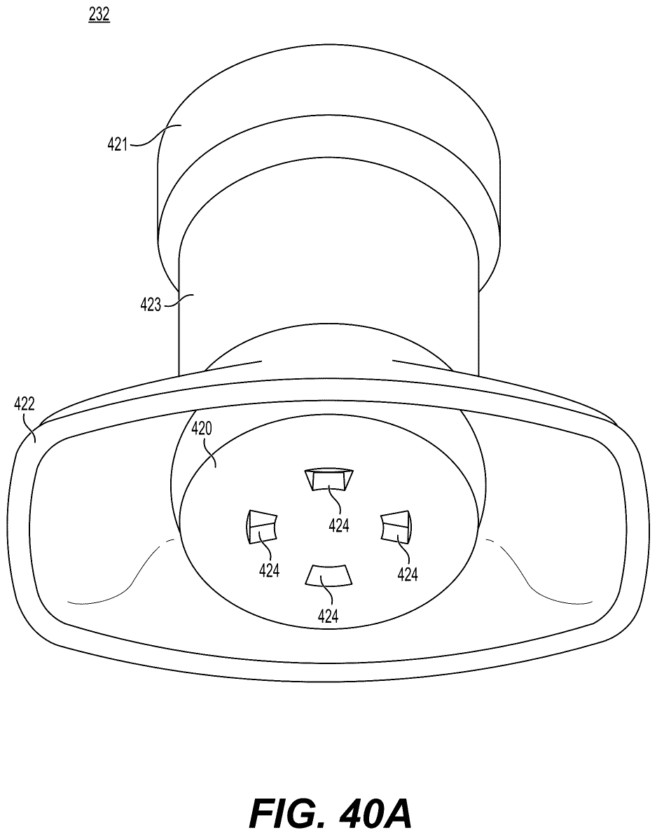

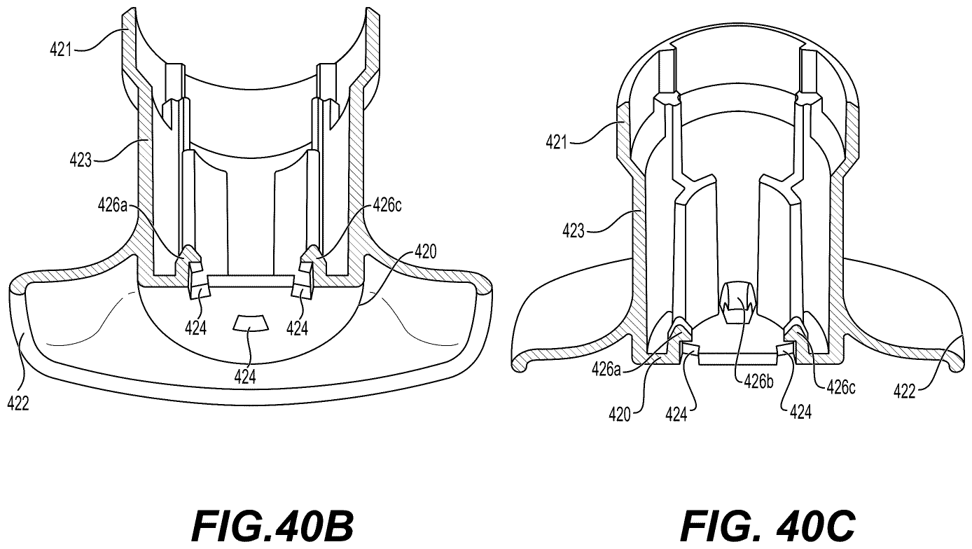

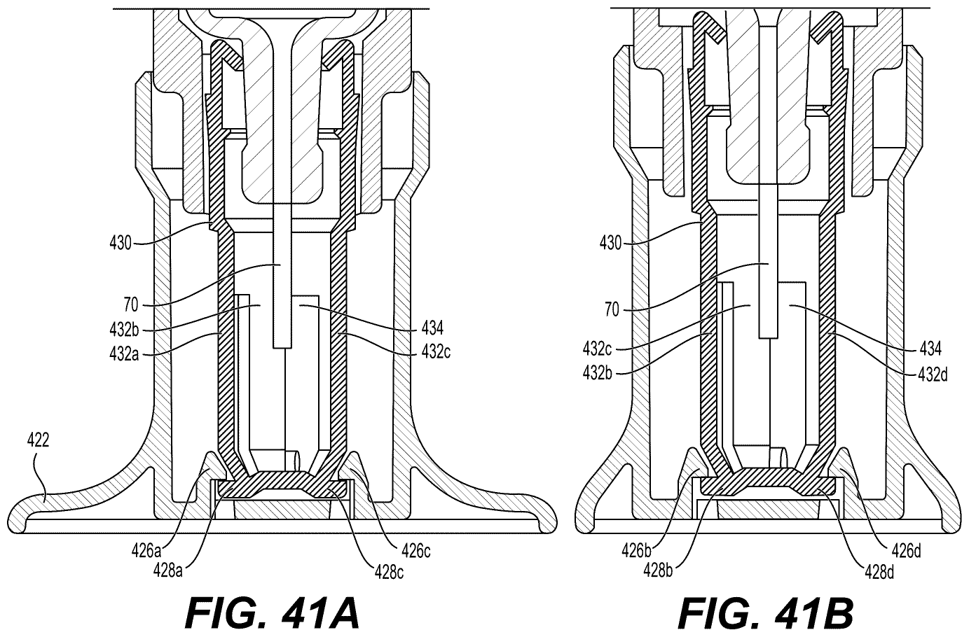

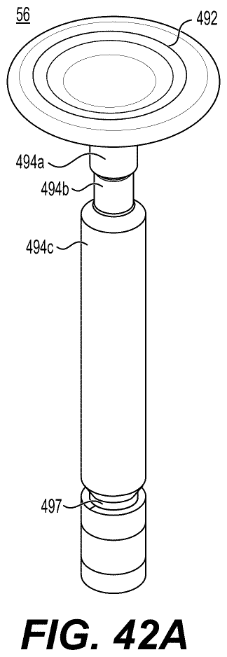

To initiate injection, needle cap 232 is removed from device 140. Removing needle cap 232 from device 140 may also remove needle shield 430. Needle cap 232 and needle shield 430 will be discussed in further detail below in regards to FIGS. 40A-41B. After needle cap 232 and/or needle shield 430 are removed from device 140, device 140 may be placed and/or pressed against an injection site. Pressing device 140 against an injection site may depress needle cover 64 in a proximal direction. In some examples, once needle cap 232 and/or needle shield 430 are removed from device 140, a distal end portion of needle 70 may protrude from a distal end of needle cover 64. In such examples, pressing device 140 against an injection site may minimally depress needle cover 64 in a proximal direction, since the distal end portion of needle 70 is already protruding from the distal end of needle cover 64. In some examples, once needle cap 232 and/or needle shield 430 are removed from device 140, needle 70 may be fully housed within needle cover 64. In such examples, pressing device 140 against an injection site may depress needle cover 64 in a proximal direction to expose a distal end of needle 70. Plunger rod 56 may then be pressed in a distal direction into housing 54. As plunger rod 56 is pressed in a distal direction into housing 54, plunger rod 56 may deflect the second pair of deflectable arms 63b away from their corresponding indents 170 of plunger rod 56 due to the increased diameter of plunger rod 56, e.g., at third portion 57c of plunger rod 56 interacting with the second pair of deflectable arms 63b. This movement of plunger rod 56 initiates injection (FIG. 36C). As plunger rod 56 continues to move in a distal direction, plunger rod 56 slides past the second pair of deflectable arms 63b. At this stage, there may be increased friction on plunger rod 56 against the second pair of deflectable arms 63b due to the radially inward extending incline, which may require deliberate motion/force by the user to continue injection. During injection, first pair of deflectable arms 63a may deflect inwards towards plunger rod 56, due to the decreased diameter of plunger rod 56, e.g., at second portion 57b of plunger rod 56, allowing each arm of the first pair of deflectable arms 63a to detach from their corresponding opening 172 (FIG. 36D). Once injection is complete, device 140 may be removed from the injection site and needle cover 64 may automatically be pulled in a distal direction such that needle cover 64 covers a portion of needle 70 (FIGS. 35A-35B). In some examples, spring shroud 53 may pull needle cover 64 in a distal direction such that needle cover 64 covers a portion of needle 70. In some examples, once injection is complete, device 140 may be removed from the injection site and needle cover 64 may automatically be pulled in a distal direction such that the entire needle 70 is housed within needle cover 64 (FIGS. 35A-35B). In some examples, spring shroud 53 may pull needle cover 64 in a distal direction such that the entire needle 70 is housed within needle cover 64.

At the end of injection, e.g., end-of-dose state or lockout state, referring to FIG. 36E, second pair of deflectable arms 63b may return to an un-deflected, e.g., relaxed position, due to plunger rod 56 being located distally within syringe 52, such that second pair of deflectable arms 63b abut second portion 57b of plunger rod 56, where second portion 57b may have a diameter that is smaller than the diameter of third portion 57c of plunger rod 56. In some embodiments, the act of second pair of deflectable arms 63b abutting second portion 57b, may produce a sound, e.g., an audible click, which may serve as an end-of-dose indicator. At this stage, second pair of deflectable arms 63b may be located proximally to needle cover 64, as shown in FIG. 36E, preventing needle cover 64 from moving in a proximal direction. At the lockout state, as shown in FIG. 36F, first pair of deflectable arms 63a may be located proximally to needle cover 64. First pair of deflectable arms 63a may return to an un-deflected, e.g., relaxed position, preventing needle cover 64 from moving in a proximal direction. First pair of deflectable arms 63a may now act as a lockout mechanism to prevent re-use of device 140 or accidental needle stick, since needle cover 64 may no longer be moved distally to expose needle 70. At this final, post-injection position of device 140, needle cover 64 may be in a lockout position and a proximal end of needle cover 64 and first pair of deflectable arms 63a may abut one another (FIG. 36F). At this stage, device 140 may not be re-used. As first pair of deflectable arms 63a relax back to their neural position, first pair of deflectable arms 63a may abut a portion of housing 54, producing a sound, e.g., an audible click, which may serve an end-of-dose indicator.

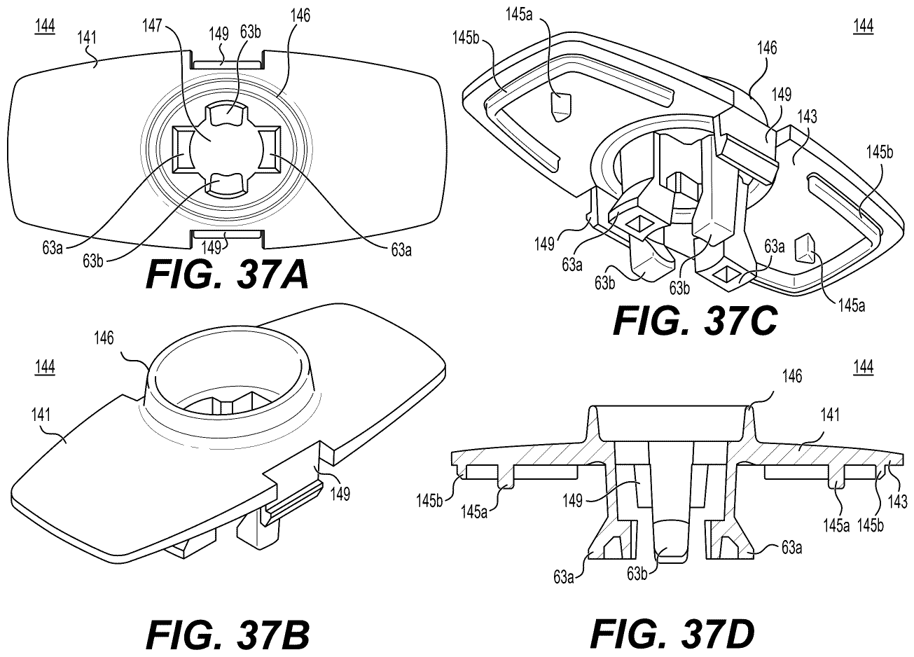

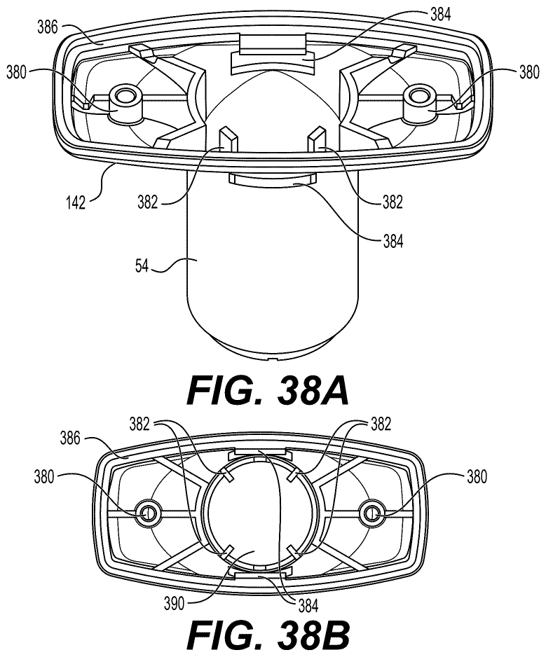

FIGS. 37A-37D depict details of rear cap 144 and FIGS. 38A-38B depict details of housing 54. Rear cap 144 may include a proximal surface 141, a distal surface 143, a collar 146 surrounding an opening 147, and a plurality of rear cap hooks 149. As described throughout the present disclosure, collar 146 may extend in a proximal direction away from proximal surface 141. Collar 146 may be any appropriate size and/or configuration to surround a portion of plunger rod 56. In some embodiments, collar 146 may have a generally circular shape. As shown in FIG. 37A, collar 146 may surround opening 147. Opening 147 may be configured to receive a portion of plunger rod 56. In some examples, opening 147 may have a generally circular shape. Plurality of rear cap hooks 149 may be any appropriate size and/or configuration to securely couple and/or permanently attach rear cap 144 to housing 54. For example, plurality of rear cap hooks 149 may be tabs that attach to a corresponding portion of housing 54, e.g., recesses 384. In some examples, rear cap 144 may include a plurality of protrusions 145a, 145b. As shown in FIGS. 37C-37D, plurality of protrusions 145a, 145b may extend in a distal direction away from distal surface 143. Plurality of protrusions 145a, 145b may have any appropriate size, shape, and/or configuration, such that plurality of protrusions 145a, 145b may securely couple and/or permanently attach to housing 54. For example, first plurality of protrusions 145a may have a generally triangular, rectangular, or cuboidal shape. The shape of first plurality of protrusions 145a may correspond to recesses 380 (FIG. 38A) in housing 54. In other examples, second plurality of protrusions 145b may fit into and/or abut a corresponding rim 386 of housing 54. Referring to FIGS. 38A-38B, housing 54 may include finger flange 142 as described in detail throughout the present disclosure and opening 388 configured to receive syringe 52. Opening 388 may have a generally circular shape. In some examples, housing 54 may include a plurality ribs 382. Each rib of plurality of ribs 382 may have a generally rectangular shape to maintain a position of syringe 52 within housing 54.

First pair of deflectable arms 63a and second pair of deflectable arms 63b are described in detail above. FIGS. 37A-37D depict exemplary configurations of first pair of deflectable arms 63a and second pair of deflectable arms 63b. For example, a first arm of the first pair of deflectable arms 63a may be located opposite from a second arm of the first pair of deflectable arms 63a. Further, a first arm of the second pair of deflectable arms 63b may be located opposite from a second arm of the second pair of deflectable arms 63b. In embodiments where there are four deflectable arms, each deflectable arm may be about 90 degrees from one another. In some embodiments, an arm of the first pair of deflectable arms 63a may alternate with an arm of the second pair of deflectable arms 63b about the circumference of opening 147.

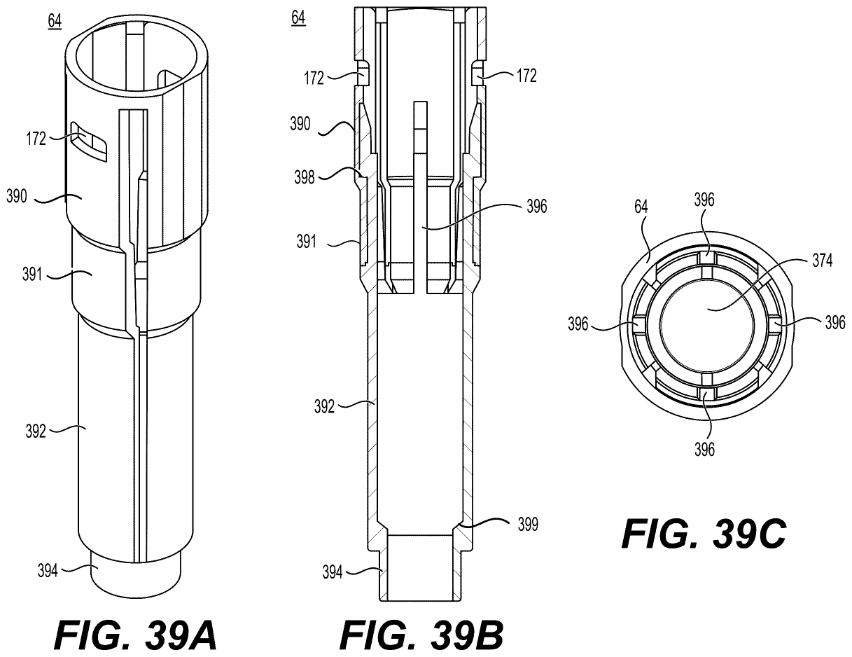

FIGS. 39A-39C depict details of needle cover 64. Needle cover 64 may include at least one opening 172, as described above in regards to FIGS. 36A-36D. As shown in FIG. 39B, needle cover 64 may include at least two openings 172. Opening 172 may have any appropriate size, shape, and/or configuration, for receiving a portion of first pair of deflectable arms 63a. In some examples, opening 172 may have a generally rectangular shape, but other suitable shapes are contemplated. In some examples, needle cover 64 may include a proximal portion 390, an indicator portion 391, a shaft 392, and a neck 394. The diameters of needle cover 64 may vary at the different sections. For example, referring to FIG. 39A, an external diameter of proximal portion 390 may be larger than each of an external diameter of indicator portion 391, an external diameter of shaft 392, and an external diameter of neck 394. In some examples, the external diameter of indicator portion 391 may be less than the external diameter of proximal portion 390 but larger than each of the external diameter of shaft 392 and the external diameter of neck 394. In other examples, the external diameter of shaft 392 may be less than each of the external diameter of proximal portion 390 and the external diameter of indicator portion 391, but greater than the external diameter of neck 394. The internal diameters may also vary at the different sections. For example, referring to FIG. 39B, an internal diameter of proximal portion 390 may be larger than each of an external diameter of indicator portion 391, an external diameter of shaft 392, and an internal diameter of neck 394. In some examples, the internal diameter of indicator portion 391 may be less than the internal diameter of proximal portion 390 but larger than each of the internal diameter of shaft 392 and the internal diameter of neck 394. In other examples, the internal diameter of shaft 392 may be less than each of the internal diameter of proximal portion 390 and the internal diameter of indicator portion 391, but greater than the internal diameter of neck 394. Still referring to FIG. 39B, proximal portion 390 may include at least one proximally facing ledge 398. In some examples, ledge 398 may support a portion of syringe 52, e.g., a proximal end portion of syringe 52. In some examples, a distal portion of shaft 392 may include at least one inclined ledge 399.

At end-of-dose, indicator portion 391 may be pushed distally out of housing 54, such that a portion of indicator portion 391 may be visible to the user. Indicator portion 391 may include a color that differs from other portions of needle cover 64 and/or device 140 and/or designs that may be visible to a user. Such indicator portion 391 may indicator end-of-dose, e.g., completion of the injection process. Needle cover 64 may also include at least one protrusion 396. As shown in FIGS. 39B-39C, needle cover 64 may include at least one protrusion 396 located on an interior surface of needle cover 64 with the length of the protrusion extending in the same direction as the length of needle cover 64. In some examples, needle cover 64 may include a plurality of protrusions 396. For example, needle cover 64 may include at least one protrusion 396, at least two protrusions 396, at least three protrusions 396, or at least four protrusions 396. Protrusions 396 may have any appropriate size, shape, and/or configuration to secure syringe 52. For example, protrusions 396 may be circumferentially spaced apart, where each of the protrusions 396 extends in the same orientation. As shown in FIG. 39C, needle cover 64 forms a periphery surrounding an interior cavity 374 configured to receive syringe 52.

FIGS. 40A-40C depict needle cap 232. Needle cap 232 may be attached to a distal end of device 140 (FIGS. 33A-33B). In some examples, needle cap 232 may include a cap collar 421, a cap neck 423, and a cap skirt 422. As shown in FIG. 40A, cap skirt 422 may surround a distal end 420 of needle cap 232. Distal end 420 may include a plurality of openings 424. For example, distal end 420 may include at least one opening 424, at least two openings 424, at least three openings 424 or at least four openings 424. Openings 424 may serve as anti-choke holes. In some examples, during manufacturing, openings 424 may be made to create clips 426a, 426b, 426c, 426d. FIGS. 40B-40C depict cut-out views of needle cap 232 and FIGS. 41A-41B depict needle cap 232 attached to a device disclosed herein, wherein needle cap 232 may be attach to a needle shield 430. FIG. 41A depicts a front cut-out view of needle cap 232 and FIG. 41B depicts a side cut-out view of needle cap 232. Needle shield 430 may extend in a distal direction to fully enclose needle 70 and/or abut a portion of needle cap 232. In some examples, needle cap 232 may include a plurality of clips 426a, 426b, 426c, 426d. In some examples, needle cap 232 may include at least one clip, at least two clips, at least three clips, or at least four clips. Each clip 426a, 426b, 426c, 426d may be shaped and/or configured to attach to, e.g., latch onto, a corresponding ledge 428a, 428b, 428c, 428d of needle shield 430. For example, clip 426a may latch onto ledge 428a, clip 426b may latch onto ledge 428b, clip 426c may latch onto ledge 428c, and clip 426d may latch onto ledge 428d. As shown in FIGS. 41A-41B, needle shield 430 may include a plurality of arms 432a, 432b, 432c, 432d. Needle shield 430 may include at least one arm, at least two arms, at least three arms, or at least four arms. In some embodiments, needle shield 430 may include plurality of arms 432a, 432b, 432c, 432d and a gap 434 between each arm. As the user pulls off needle cap 232 in a distal direction away from housing 54, needle shield 430 may also simultaneously pull out of housing 54, to expose needle 70.

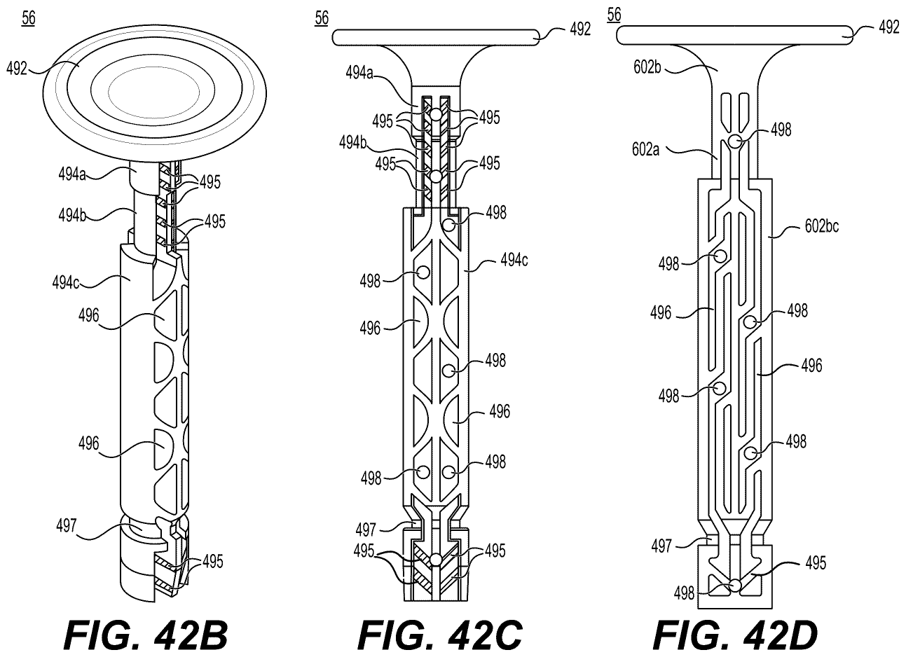

FIGS. 42A-42D depict details of plunger rod 56. Plunger rod 56 may include a thumb pad 492 for user to depress plunger rod 56 in a distal direction. In some embodiments, and as discussed above in reference to FIGS. 35A-35B, plunger rod 56 may include a first portion 494a having a first diameter, a second portion 494b having a second diameter, and a third portion 494c having a third diameter. As shown in FIG. 42A, the first diameter of first portion 494a and the third diameter of third portion 494c may each be greater than the second diameter of second portion 494b. Plunger rod 56 may include an indent 497, which may correspond to indent 170 as described above in reference to FIG. 36B. Plunger rod 56 may have various configurations, for example, as shown in FIGS. 42B-42D. For example, portions of plunger rod 56 may be cored out to form a plurality of ribs 495 and/or grooves 496. The plurality of ribs 495 and/or grooves 496 may form locations for placement of ejector pins (498). The placement of ejector pins 498 may affect the injection force as plunger rod 56 slides past components of the devices discussed herein, e.g., cap 144. To decrease the injection force, ejector pins 498 may be placed in areas of the device other than the external surface of plunger rod 56. For example, ejector pins 498 may be placed in the internal surface of plunger rod 56. FIG. 42D depicts another exemplary plunger rod 56 including a first portion 602a having a first diameter and second portion 602b having a second diameter. In some examples, the second diameter of second portion 602b may be greater than the first diameter of first portion 602a. Plunger rod 56 as depicted in FIG. 42D may include a plurality of ribs 495 and/or grooves 496.

Embodiments of device 140 and components depicted in FIGS. 14A-16B and FIGS. 33A-42D may include one or more additional features, e.g., optimal configuration and sizing of deflectable arms 63a, 63b; ergonomic design to enable two hand operation, e.g., one hand may hold the syringe for needle insertion at 90 degrees, while the second hand pushes plunger rod 56; indicators of injection completion, e.g., a visual color change or audible indicator; activation points to mitigate potential failure modes, e.g., a pre-activation point or state; extension of plunger rod 56 to sit within a piston of the device to improve alignment of plunger rod 56 before and/or during use; and optimization of the design to reduce the required spring force to minimize plastic creep.

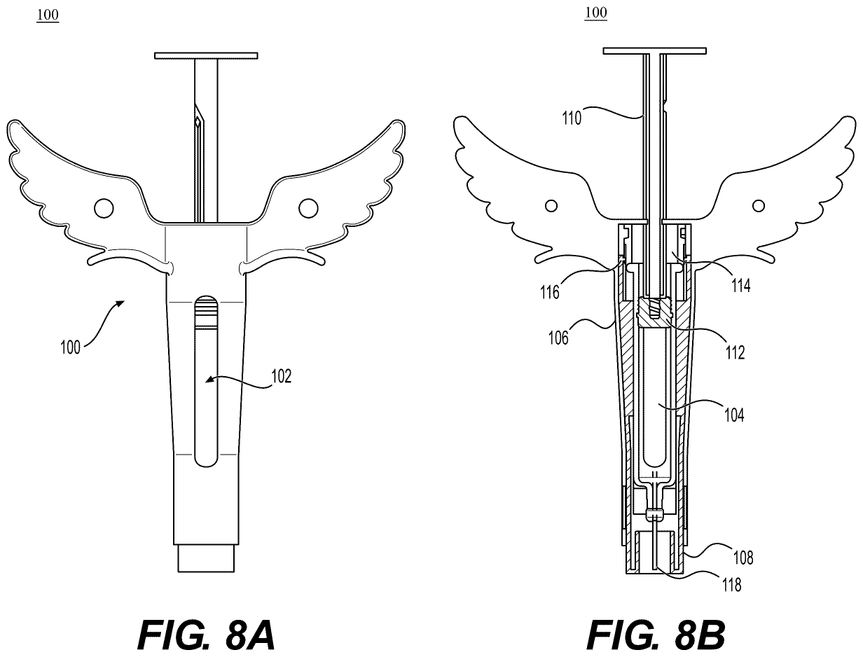

Another embodiment of the present disclosure is shown in FIGS. 8A-10B. FIG. 8A shows an exterior view of a device 100, while FIG. 8B shows a cross section of the device. Device 100 may be designed to hold any suitable commercially known product container 104 (e.g., a syringe or other suitable drug delivery device). Referring to FIG. 8B, device 100 may include a housing 106, needle cover 108, plunger rod 110, stopper 112, and rotating component 114, e.g., a cam.

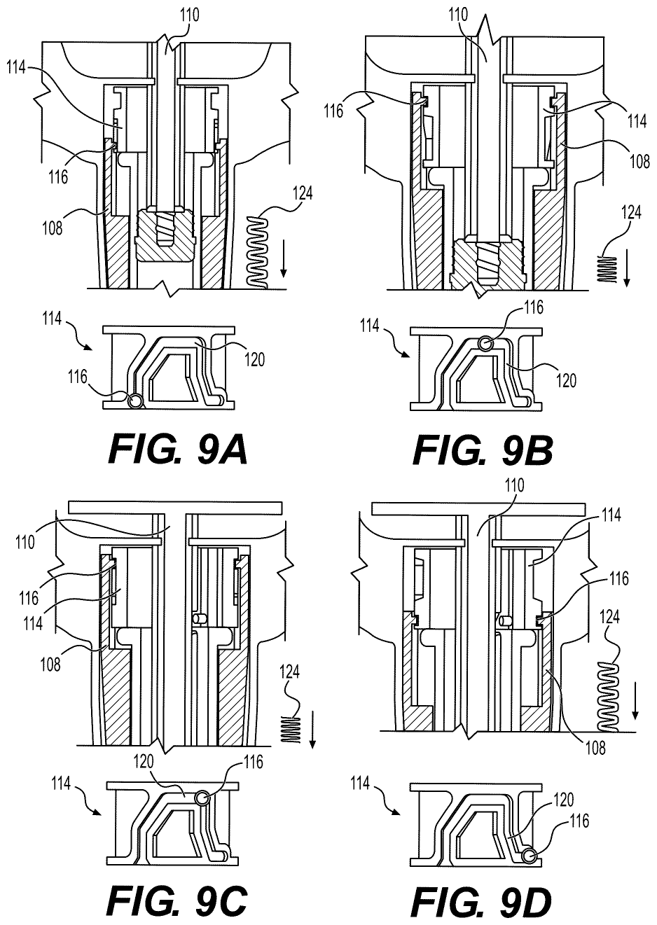

Prior to injection, needle cover 108 may extend distally from housing 106 to cover needle 118. To initiate injection, device 100 may be pressed against an injection site. Device 100 may include a spring (not shown), e.g., around or at an end of needle cover 108. An exemplary actuator, e.g., spring, 124 is shown schematically in FIGS. 9A-9D for discussion purposes. An upper region of each of FIGS. 9A-9D depicts a cross section of plunger rod 110, rotating component 114, and protrusion 116 of needle cover 108 that interacts with track 120 of rotating component 114. A bottom region of each of FIGS. 9A-9D depicts a side view of movement of protrusion 116 of needle cover 108 along track 120 of rotating component 114 that corresponds to the cross-sectional view of that figure.

As shown in FIGS. 9A-9D, rotating component 114 may include a track 120, e.g., a groove, for receiving a protrusion 116 of needle cover 108. At an initial position (FIG. 9A), actuator 124 may be uncompressed, needle cover 108 may extend distally over needle 118, and plunger rod 110 may be locked in position relative to rotating component 114. This may prevent plunger rod 110 from being pushed distally into housing 106 until needle cover 108 is pushed proximally to reveal needle 118. To initiate injection, plunger rod 110 may be twisted relative to rotating component 114, allowing plunger rod 110 to move from a locked position to an unlocked position. Needle cover 108 may be pushed proximally into housing 106, e.g., by a user pressing device 100 against an injection site, which may cause rotating component 114 to turn and actuator 124 to compress during injection (FIG. 9B). Plunger rod 110 may be locked in place, e.g., movement of plunger rod 110 may be prevented, until needle cover 108 is retracted proximally into housing 106. Such configuration may prevent premature expulsion of the product. During injection, needle cover 108 may be held in the retracted position due to the location of protrusion 116 on needle cover 108 in track 120 of rotating component 114 (FIG. 9B). This may act as a signal to the user that device 100 is still in use. In this retracted position, needle cover 108 may also control insertion depth of needle 118, since a distal end of needle cover 108 may prevent deeper insertion of needle 118 once the distal end of needle cover 108 contacts the injection site. Once injection is complete, plunger rod 110 may turn rotating component 114 to free needle cover 108 (FIG. 9C), and actuator 124 may push needle cover 108 back down to a lockout position, wherein needle cover 108 extends distally from housing 106 (FIG. 9D) to cover needle 118 once the injection is complete. As can be seen in FIG. 9D, in the lockout position, at the end of the injection, protrusion 116 on needle cover 108 may engage a notch at the end of track 120 on rotating component 114 that prevents needle cover 108 from being pushed proximally into housing 106 again to prevent re-use of device 100.

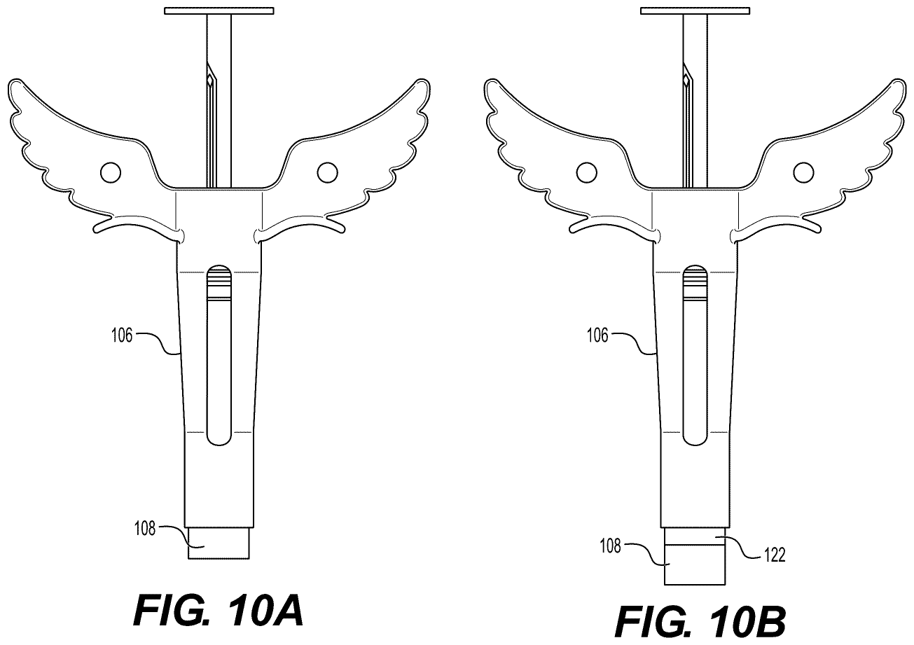

In some embodiments, in the lockout position, needle cover 108 may be pushed out farther distally than the initial position of needle cover 108. For example, FIG. 10A depicts an initial position of needle cover 108 prior to injection, and FIG. 10B depicts a lockout position of needle cover 108 at end-of-dose. Portion 122 represents an additional portion of needle cover 108 that may be exposed at the end of the injection, but not at the starting point of the injection. In some embodiments, the height of track 120 rotating component 114 may be greater at the end compared to the beginning of track 120. For example, the beginning of track 120 (shown at the left side of rotating component 114 in FIGS. 9A-9D) may start at a central region of rotating component 114, may continue to an upper region of rotating component 114, and then the end of track 120 (shown at the right side of rotating component 114 in FIGS. 9A-9D) may be located at a lower region of rotating component 114. Accordingly, the end of track 120 may have a height greater than a height of the initial part of track 120. This increased height at the end of track 120 may allow needle cover 108 to extend out further at its lockout position than at its original position (FIG. 10B).

The embodiments of FIGS. 8A-10B depict a device with a passive safety mechanism that may activate when the user nears end-of-dose, e.g., when a substantial amount of the product is fully expelled from container 104. The passive safety mechanism of FIGS. 8A-10B may allow for plunger rod 110 to turn rotating component 114 when end-of-dose is approach, which may push needle cover 108 proximally to an extended lockout position. In the lockout position, needle cover 108 may not retract back into housing 106. In such a lockout position, as well as prior to injection, needle cover 108 may cover needle 118 to prevent accidental needle stick post-injection. This may also reduce needle-phobia and needle-related anxiety as compared to conventional drug delivery devices with exposed needles. In addition, needle cover 108 may allow for controlled needle insertion depth. In other words, exposing only a portion of needle 118 during injection may allow for usage of device 100 without any pre-injection steps, e.g., pinching the skin to alter the injection depth to compensation for a needle length that is longer compared to the exposed needle 118 of device 100. Device 100 may be configured, e.g., container 104 may be positioned within housing 106 and/or needle cover 108 may be sized, so that needle cover 108 exposes only a portion of needle 118, and the length of the exposed portion of needle 118 represents the desired insertion depth of needle 118. As discussed above, during injection, needle cover 108 may be held back in a retracted position (FIG. 9B) due to the position of protrusion 116 on needle cover 108 in track 120 of rotating component 114. This may prevent the passive safety mechanism from activating prior to end-of-dose. Additionally, if device 100 is removed from the injection, accidentally or on purpose, passive safety mechanism may not activate, as needle cover 108 is held in track 120. Once device 100 is reapplied to the injection site and plunger rod 110 may be depressed, injection may resume.

In some embodiments, device 100 may include a feedback mechanism. Device 100 may include a visual feedback mechanism, e.g., plunger rod 110 abutting housing 106, needle cover 108 fully extended distally from housing 106, and/or an extended portion 122 distal of housing 106 (FIG. 10B). In some examples, needle cover 108 may have a first color, pattern, or texture, e.g., and extended portion 122 may have a second color, pattern, or texture, to show that device 100 is in the lockout position, and the device has been used and the dose has been expelled. In some examples, device 100 may include an opening 102 for viewing container 104 and movement of plunger rod 110 and/or stopper 112 in container 104. Device 100 may also include an audible feedback mechanism, e.g., an audible “clicking” sound once plunger rod 110 abuts housing 106 and/or needle cover 108 moves distally at the end of the dose. In some aspects, device 100 may include a tactile feedback, e.g., a snap or vibration as needle cover 108 moves distally at the end of the injection.

Device 100 may be of any suitable size and shape to hold or partially hold a container 104, and/or to support and hold any components described herein. Any components of device 100 may be of a suitable size and/or shape to configure to and cooperate with other components as designed. For example, rotating component 114 may be of any suitable size and/or shape to slidably couple to plunger rod 110. In another example, needle cover 108 and/or protrusion 116 may be of any suitable size and/or shape to fit into track 120 of rotating component 114.

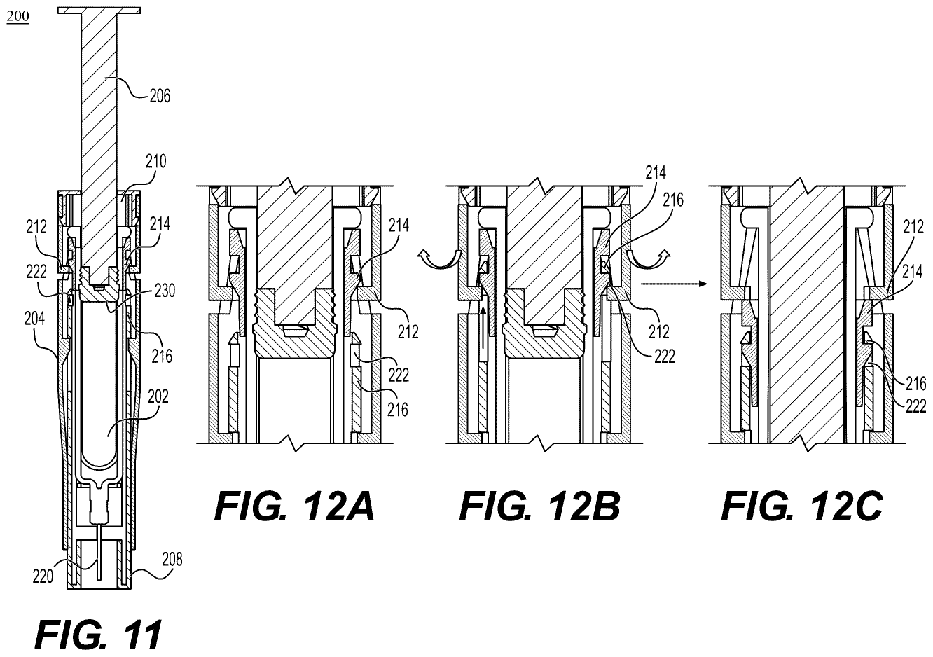

Another embodiment of the present disclosure is shown in FIGS. 11-13B. FIG. 11 shows an exterior view of a device 200. Device 200 may be designed to hold any suitable commercial product container 202 (e.g., a syringe or other suitable drug delivery device). Referring to FIG. 11, device 200 may include a housing 204, plunger rod 206, needle cover 208, and stopper 230. Proximal region of housing 204 may comprise one or more deflectable housing tabs 212. Housing 204 may also include a housing cap 210 located within a proximal portion of housing 204. Device 200 may include a lockout ring 214 slidably coupled to a proximal end of container 202. In some embodiments, housing 204 may include more than one deflectable housing tabs 212. For example, housing 204 may include two, three, four, or more deflectable housing tabs 212. In configurations with more than one deflectable housing tabs 212, the deflectable housing tabs 212 may be equally spaced apart from one another. For example, if the housing 204 includes four deflectable housing tabs 212, each of the deflectable housing tabs 212 may be about 90 degrees from one another. If the housing 204 includes two deflectable housing tabs 212, each of the deflectable housing tabs 212 may be about 180 degrees from one another.

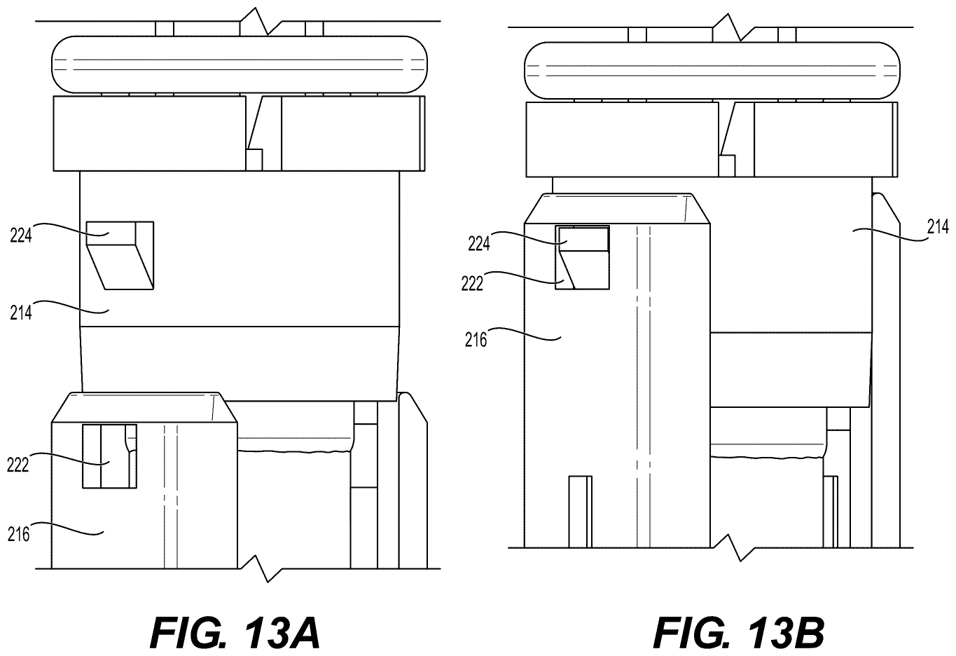

As shown in FIG. 11, needle cover 208 may be extended and covering needle 220 prior to use. At an initial position (FIG. 12A), deflectable housing tab 212 may abut a portion of lockout ring 214, and lockout ring 214 may be located proximally to a proximal end 216 of needle cover 208. The needle cover may be biased, e.g., by a spring, in the distal direction, but may be pushed back proximally within housing 204 for use. To initiate injection, needle cover 208 may be pushed in a proximal direction into housing 204, e.g., by pushing device 200 against an injection site. When needle cover 208 is moved proximally, proximal end 216 of needle cover 208 may hook onto a ledge 224 of lockout ring 214 (FIG. 12B). Movement of proximal end 216 of needle cover 208 relative to lockout ring 214 is shown in further detail in FIGS. 13A-13B. In some examples, proximal end 216 may include a cutout 222 for engaging with ledge 224. In some examples, needle cover 208 may include more than one cutout 222, e.g., a pair of cutouts, and lockout ring 214 may include more than one ledge 224, e.g., a pair of ledges. The number of ledges and cutouts may correspond with one another, so that each ledge may engage with each cutout. Although lockout ring 214 is depicted as including one or more ledges 224, and proximal end 216 of needle cover 208 is depicted as including one or more cutouts 222, the two mating regions may be reversed, e.g., lockout ring 214 may include one or more cutouts 222, and needle cover 208 may include one or more ledges 224. Further, ledges 224 and cutouts 222 may represent any suitable mating portions.

With needle cover 208 pushed proximally into housing 204, e.g., by pressing device 200 against an injection site, plunger rod 206 may be pushed in a distal direction into housing 204 to expel a product from container 202 into the injection site.

The movement of proximal end 216 of needle cover 108 into housing 204 may flex deflectable tab 212 outwards away from plunger rod 206 during injection, as shown by the arrows pointing radially outwards in FIG. 12B. Once device 200 is removed from the injection site, e.g., when injection is complete, needle cover 208, along with lockout ring 214, may be biased to retreat back downwards to its initial position, e.g., via a spring force of a spring shroud (not shown) around needle cover 208. Needle cover 208 and lockout ring 214, connected to one another via cutouts 222 and ledges 224, may move downwards past deflectable tab 212, allowing deflectable tab 212 to flex back to its initial position (FIG. 12C). As shown in FIG. 12C, a proximal end of lockout ring 214 may abut a distal end of deflectable tab 212. At this final, end of dose position, needle cover 208 may be in a lockout position, where needle cover 208 may be prevented from moving proximally back into housing 204 by deflectable tab 212, which may obstruct its path.

The embodiments of FIGS. 11-13B depict a device with a passive safety mechanism that may activate at any point when device 200 is removed from the injection site, whether or not the entire dose has been expelled from container 202. Once needle cover 208 has been pushed proximally into housing 204 to initiate the injection, the passive safety mechanism may be activated. Since needle cover 208 is biased in the distal direction, once the force pushing needle cover 208 into housing 204 is removed (e.g., by removal of device 200 from a user's skin), the safety mechanism may be activated. Such a passive safety mechanism may be helpful for users who may be concerned about needle safety. For example, if a user accidentally drops device 200 during use, the passive safety mechanism may push needle cover 208 out to prevent accidental needle stick. Additionally, needle 220 may be covered by needle cover 208 and hidden from view prior to, during, and post injection. This may reduce needle-phobia and needle-related anxiety as compared to conventional drug delivery devices with exposed needles. In addition, needle cover 108 may allow for controlled needle insertion depth. In other words, exposing only a portion of needle 220 during injection may allow for usage of device 200 without any pre-injection steps, e.g., pinching the skin to alter the injection depth to compensate for a needle length that is longer compared to the exposed needle 220 of device 200. Device 200 may be configured, e.g., container 202 may be positioned within housing 204 and/or needle cover 208 may be sized, so that needle cover 208 exposes only a portion of needle 220, and the length of the exposed portion of needle 220 represents the desired insertion depth of needle 220.

In some embodiments, device 200 may include a feedback mechanism. Device 200 may include a visual feedback mechanism, e.g., plunger rod 206 abutting housing 204, needle cover 208 fully extended distally from housing 204. In some examples, device 100 may include an opening for viewing container 202 and movement of plunger rod 206 and/or stopper 230 in container 202. Device 200 may also include an audible feedback mechanism, e.g., an audible “clicking” sound once plunger rod 206 abuts housing 204, when needle cover 208 extends distally, and/or when deflectable tab 212 relaxes back to its initial, neutral position (FIG. 12C). In some aspects, device 200 may include a tactile feedback, e.g., a snap or vibration as needle cover 208 moves distally.

Device 200 may be of any suitable size and shape to hold or partially hold a product container 202, and/or to support and hold any components described herein. Any components of device 200 may be of a suitable size and/or shape and configured to cooperate with other components as designed. For example, needle cover 208, proximal end 216, cutout 222, lockout ring 214, and ledge 224, may be of any suitable size and/or shape allowing for cutout 222 to lock onto ledge 224.

FIGS. 22A and 22B depict embodiments of needle shield 94 and needle cap 232 that may be used with any of the devices disclosed herein. Needle shield 94 may attach to a needle cover (not shown) and may extend in a distal direction to fully enclose needle 96. As shown in FIGS. 22A and 22B, needle shield 94 may extend in a distal direction such that needle shield 94 may abut a portion of needle cap 232. Needle cap 232 may include a clip 221. In other embodiments, needle cap 232 may include a plurality of clips 221. Clip 221 may be shaped or configured to attach to, e.g., latch onto, a ledge 223 of needle shield 94. Needle cap 232 as depicted in FIGS. 22A and 22B show an arm 95 of needle shield 94. In other embodiments, needle shield 94 may include a plurality of arms 95 and a gap between each arm 95. In some examples, needle cap 232 may include at least two clips, at least three clips, or at least four clips. Referring to FIG. 22B, a first clip 221a may attach onto a first ledge 223a and abut a first arm 95 of needle shield 94. A second clip 221b may attach to a second ledge 223b. As the user pulls off needle cap 232 in a distal direction away from housing 14, needle shield 94 may also simultaneously out of housing 14, to expose needle 86.

Additional concepts and features that may be used with or in any of the devices disclosed herein will be discussed below. The following concepts and features may correspond to concepts and features as discussed throughout the present disclosure.

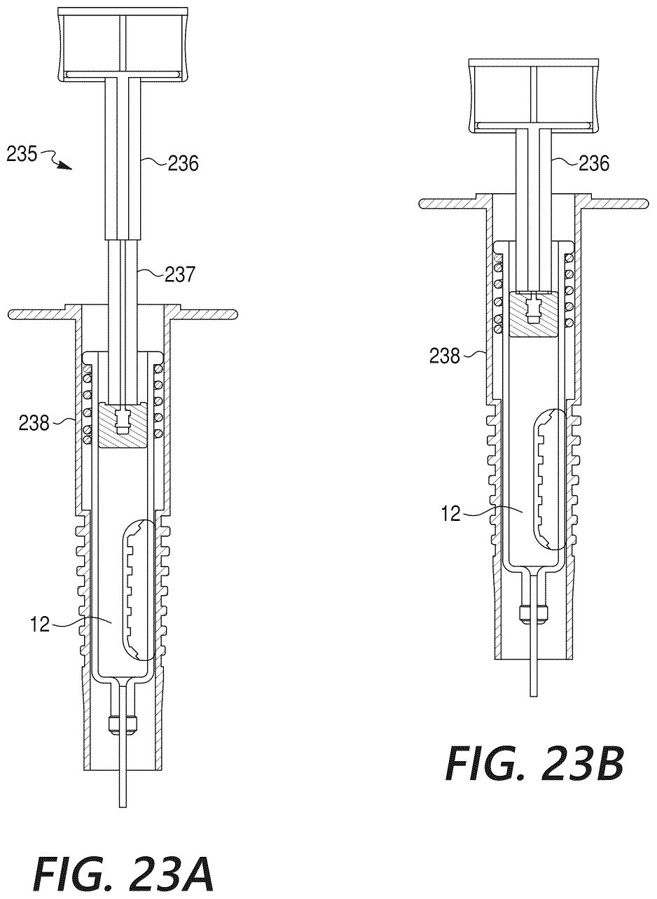

FIGS. 23A and 23B depict a plunger rod 235 that may be utilized in any of the embodiments discussed herein. Plunger rod 235 may include a first portion 236 and a second portion 237. As shown in FIG. 23A, second portion 237 may have a width that is less than a width of first portion 236. Referring to FIG. 23B, as plunger rod 235 moves distally toward housing 238, dispensing a medicament out of container 12, second portion 237 may collapse into, e.g., insert into, first portion 236. In other embodiments, first portion 236 may have a width that is less than width of second portion 237, such that as plunger rod 235 moves distally toward housing 238, first portion 236 may collapse into second portion 237. Plunger rod 235 may be referred to as a telescoping plunger rod. As shown in FIGS. 23A and 23B, as plunger rod 235 collapses, a length of plunger rod 235 decreases from a first length (FIG. 23A) to a second length (23B).

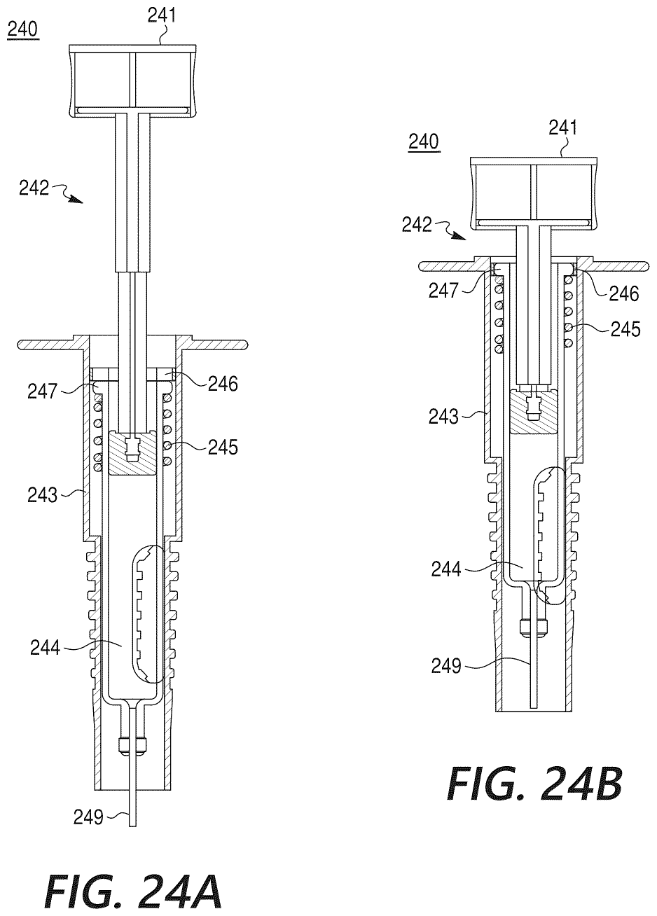

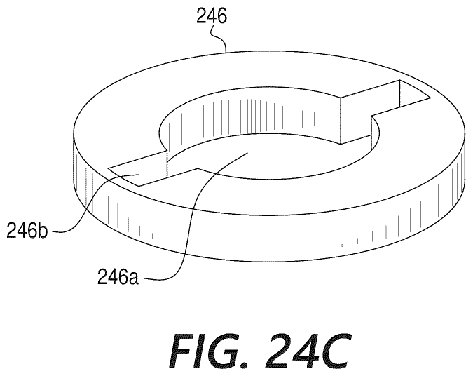

FIGS. 24A-24C depict an embodiment of the present disclosure. Device 240 may include a thumb pad 241, a plunger rod 242, a housing 243, a syringe 244, a spring shroud 245, a rotator 246, and a needle 249. In a pre-injection state, as depicted in FIG. 24A, rotator 246 may be located proximally to a proximal end of syringe 244. Referring to FIG. 24C, rotator 246 may include an opening 246a shaped and configured to surround a portion of plunger rod 242. In some examples, opening 246a may have a circular shape such that a portion of plunger rod 242 may slide in a distal direction through rotator 246. Rotator 246 may also include a plurality of cutouts 246b. Cutouts 246b may have a shape, size, and configuration, corresponding to a proximal end of syringe 244. For example, cutouts 246b may correspond to a flange 247 of syringe 244. In a pre-injection state, rotator 246 may be located proximally to a proximal end of container 12 such that cutouts 246b are displaced from flange 247. Spring shroud 245 may surround a proximal end of syringe 244, such that syringe 244 may be spring loaded and held down by rotator 246 (FIG. 24A). To initiate injection, a user may depress thumb pad 241 in a distal direction towards housing 243. During injection, as plunger rod 242 moves in a distal direction into housing 243 and syringe 244, plunger rod 242 may rotate such that cutouts 246b rotate towards flange 247. At end-of-dose, as depicted in FIG. 24B, syringe 244 may move in a proximal direction into housing 243 and into rotator 246, and needle 249 may be contained within a distal end of housing 243. In some embodiments, rotator 246 and syringe 244 may not be coupled together and then may move proximally towards a proximal end of housing 243. Any appropriate syringe 244 may be used with device 240. For example, syringe 244 may be a cyclo-olefin-polymer syringe or a glass cut flange syringe.

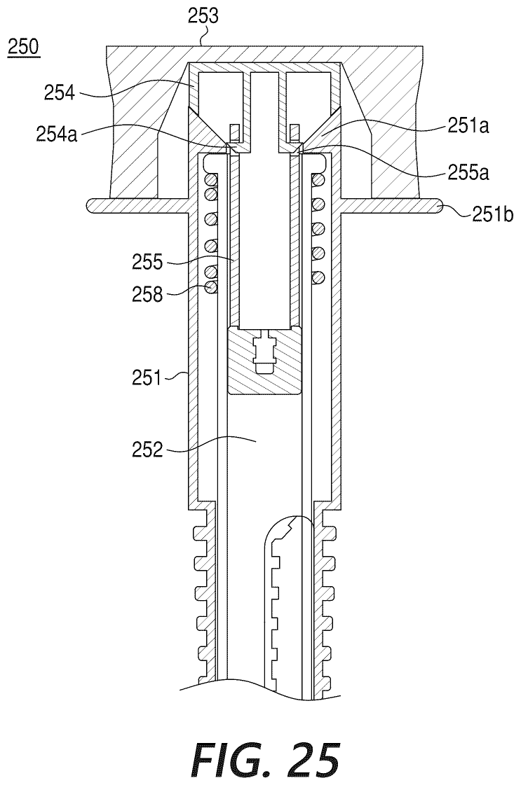

FIG. 25 depicts an embodiment of the present disclosure. Device 250 may include a housing 251, a syringe 252, and a thumb pad 253. Device 250 may also include a telescoping plunger rod including a first portion 254 and a second portion 255. The telescoping plunger rod may correspond to plunger rod 235 depicted in FIGS. 23A and 23B. As shown in FIG. 25, first portion 254 may include plurality of ledges 254a and second portion 255 may include a plurality of cutouts 255a. Ledges 254a may fit into cutouts 255a, coupling first portion 254 to second portion 255. At end-of-dose, thumb pad 253 may abut a flange portion 251b of housing 251. Housing 251 may include distal extensions 251a. Referring to FIG. 25, at end-of-dose, once thumb pad 253 abuts flange portion 251b, first portion 254 and second portion 255 may also abut distal extensions 251a. As first portion 254 and second portion 255 abut distal extensions 251a, ledges 254a may push against distal extensions 251a, deflecting distal extensions 251a outwards and away from syringe 252. Spring shroud 258 may then draw syringe 252 and/or first portion 254 proximally into thumb pad 253, drawing a needle at the distal end of the syringe (not shown) into housing 251. In some embodiments, thumb pad 253 and housing 251 may include attachment features for locking thumb pad 253 and housing 251 together at the end-of-dose stage.

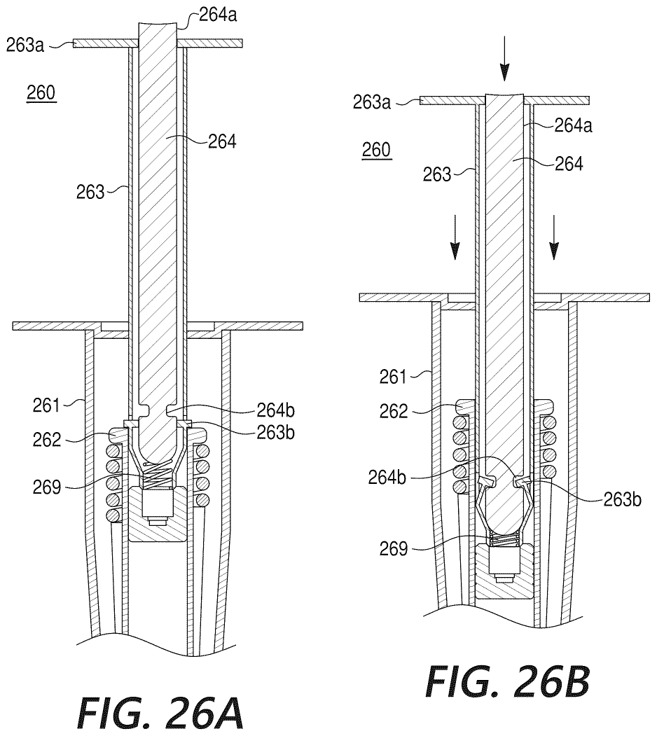

FIGS. 26A and 26B depict an embodiment of the present disclosure. Device 260 may include a housing 261, a syringe 262, and a plunger rod 263. Device 260 may also include an internal rod 264 extending through plunger rod 263. As shown in FIG. 26A, internal rod 264 may include a button 264a extending proximally from a thumb pad 263a of plunger rod 263. In a pre-injection state, deflectable arms 263b of plunger rod 263 may abut against a distal portion of internal rod 264, preventing movement of plunger rod 263 and as such, preventing pre-expulsion of the contents of syringe 262. A spring 269 may be included in plunger rod 263. To initiate injection, a user may press button 264a while simultaneously pushing thumb pad 263a in a distal direction towards housing 261 and against spring 269. Pressing button 264a may move internal rod 264 in a distal direction towards housing 261. Referring to FIG. 26B, as internal rod 264 moves distally into housing 261, deflectable arms 263b may then deflect inwards into indents 264b. Once deflectable arms 263b are deflected into indents 264b, plunger rod 263 may freely move in a distal direction to dispense the contents of syringe 262.

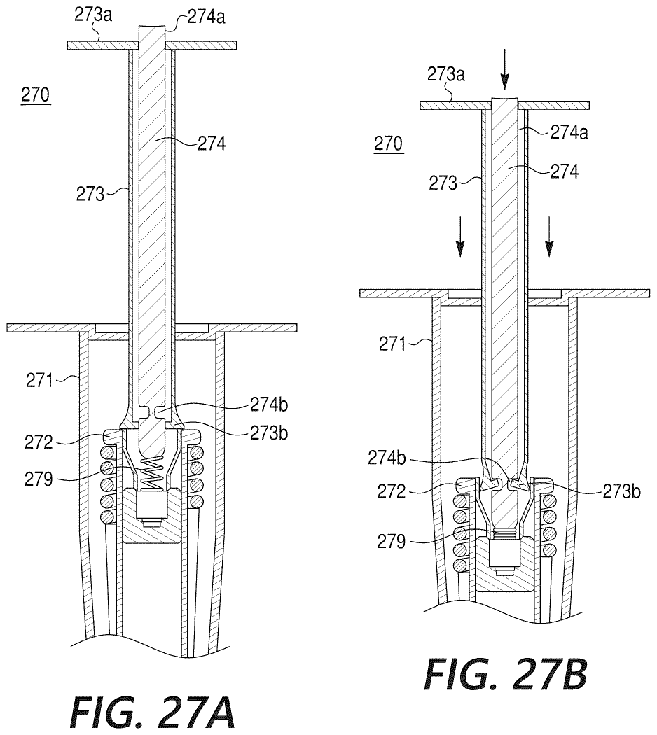

FIGS. 27A and 27B depict a device 270 similar to device 260 in FIGS. 26A and 26B. Device 270 may include a housing 271, a syringe 272, and a plunger rod 273. Device 270 may also include an internal rod 274. As shown in FIG. 27A, internal rod 274 may include a button 274a extending proximally from a thumb pad 273a of plunger rod 273. In a pre-injection state, deflectable arms 273b of plunger rod 273 may abut against a distal portion of internal rod 274, preventing movement of plunger rod 273 and as such, preventing pre-expulsion of the contents of syringe 272. A spring 279 may be included in plunger rod 273. To initiate injection, a user may press button 274a while simultaneously pushing thumb pad 273a in a distal direction towards housing 271 and against spring 279. Pressing button 274a may move internal rod 274 in a distal direction towards housing 271. Referring to FIG. 27B, as internal rod 274 moves distally into housing 271, deflectable arms 273b may then deflect inwards into indents 274b. Once deflectable arms 273b are deflected into indents 274b, plunger rod 273 may freely move in a distal direction to dispense the contents of syringe 272.

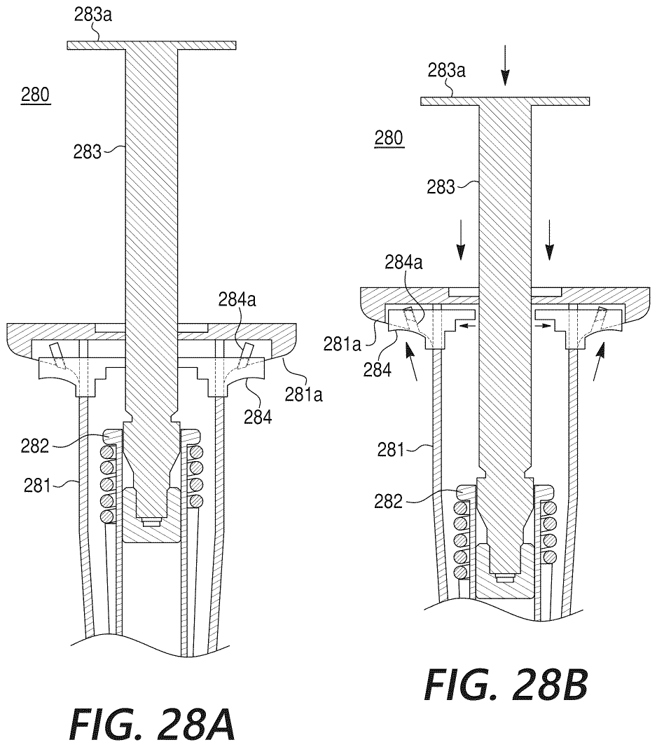

FIGS. 28A and 28B depict another embodiment according to the present disclosure. Device 280 may include a housing 281, a syringe 282, and a plunger rod 283. As shown in FIGS. 28A and 28B, housing 281 may include a flange 281a. Housing 281 may also include a trigger 284. FIGS. 28A and 28B show an interior view of device 280. Trigger 284 may extend through an internal portion of housing 281, for example, flange 281a. Referring to FIG. 28A, a portion of trigger 284 may extend distally from flange 281a. Flange 281a may include a plurality of slots 284a. Slots 284a may move trigger 284 within flange 281a. In a pre-injection state, as shown in FIG. 28A, a portion of trigger 284 abuts against plunger rod 283, preventing pre-expulsion of the contents of syringe 282. To initiate injection, a user may push thumb pad 283a distally towards housing 281, while simultaneously squeezing a portion of trigger 284 that extends distally from flange 281a. For example, the user may squeeze the portion of trigger 284 in a proximal direction into flange 281a. As the portion of trigger 284 is squeezed, slots 284a may guide trigger 284 in a direction away from plunger rod 283, allowing plunger rod 283 to move in a distal direction into housing 281 (FIG. 28B).

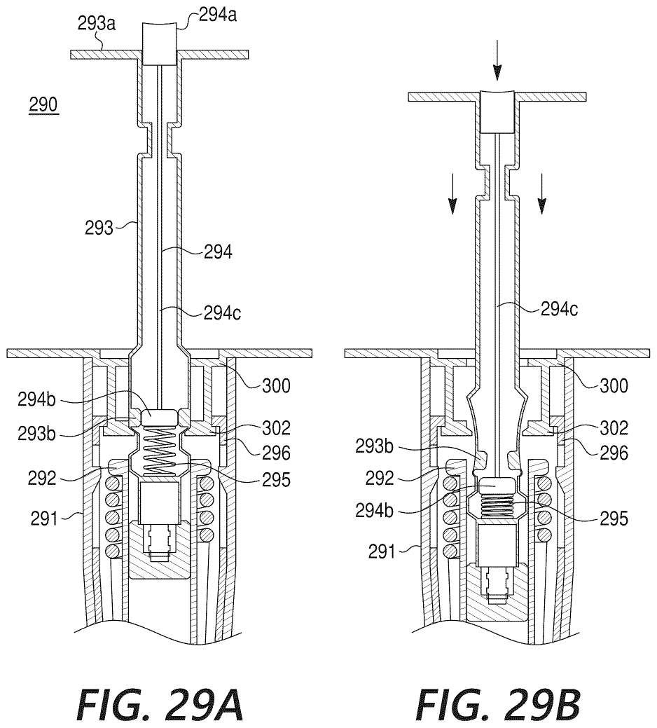

FIGS. 29A and 29B depict another embodiment according to the present disclosure. Device 290 may include a housing 291, syringe 292, plunger rod 293, and internal rod 294. As shown in FIG. 29A, device 290 may include a blocking component 300. Blocking component 300 may correspond to blocking components 62 and 82 as described herein. Blocking component 300 may include deflectable arms 302. Deflectable arms 302 may correspond to deflectable arms 63 and 83 as described herein. Internal rod 294 may include a button 294a extending proximally from thumb pad 293a of plunger rod 293. Internal rod 294 may also include a distal block 294b. In a pre-injection state, as shown in FIG. 29A, distal block 294b may be positioned above a spring 295 and within a flexible portion 293b of plunger rod 293. Distal block 294b may abut against flexible portion 293b, preventing movement of deflectable arms 302. This pre-injection state may prevent pre-expulsion of contents from syringe 292. To initiate injection, a user may press button 294a distally into plunger rod 293. Pressing button 294a may move internal rod 294 and distal block 294b in a distal direction towards syringe 292 and against spring 295. As shown in FIG. 29B, distal block 294b may be moved in a distal direction away from flexible portion 293b of plunger rod 293. Deflectable arms 302 may then deflect inwards towards flexible portion 293b, which may then allow plunger rod 293 to move distally into housing 291 and syringe 292 to expel the contents of syringe 292.

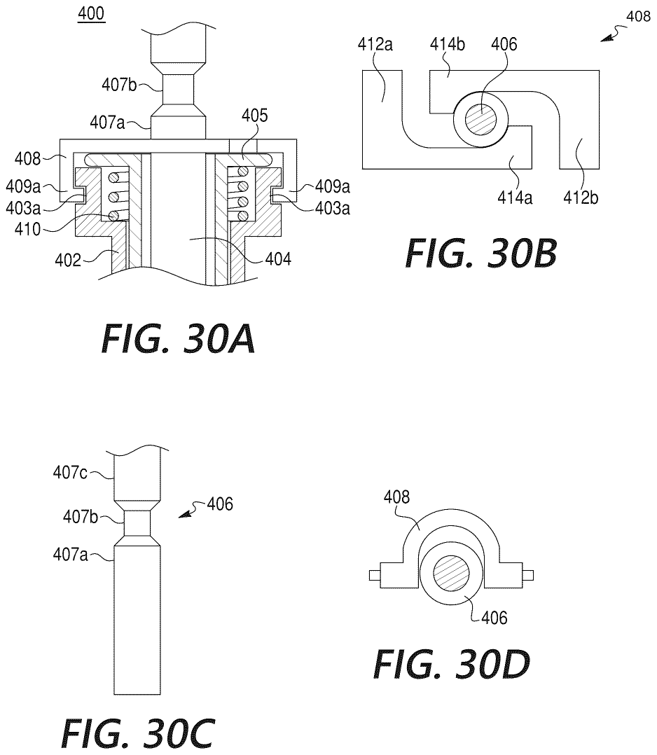

FIGS. 30A-30D depict another embodiment according to the present disclosure. Device 400 may include a housing 402, a syringe 404, a plunger rod 406, a finger flange 408, and a spring 410 surrounding a proximal portion of syringe 404. As shown in FIG. 30A, a proximal portion of housing 402 may include a plurality of recesses 403a, 403b and finger flange 408 may include a plurality of distal arms 409a, 409b. Recesses 403, 403b may be configured to receive distal arms 409a, 409b. To couple finger flange 408 to housing 402, a first distal arm 409a may hook into recess 403a and a second distal arm 409b may hook into recess 403b. Syringe 404 may also include a syringe flange 405. As shown in FIG. 30A, syringe flange 405 may sit between a portion of finger flange 408 and a portion of housing 402. For example, a proximal end of syringe flange 405 may abut a distal interior surface of finger flange 408 and a distal end of syringe flange 405 may abut a proximal end of housing 402. Referring to FIGS. 30A and 30C, plunger rod 406 may include a first portion 407a and a second portion 407b. First portion 407a may have a first diameter and second portion 407b may have a second diameter. The first diameter may be larger than the second diameter. In other examples, the first diameter may be smaller than the second diameter. In some examples, the plunger rod may have a first width and a second width, wherein the first width may be larger than the second width. In other examples, the first width may be smaller than the second width. As shown in FIG. 30C, plunger rod 406 may include a third portion 407c, wherein a diameter of third portion 407c is larger than the diameter of second portion 407b. Finger flange 408 may be any appropriate size, shape, and/or configuration to surround a portion of plunger rod 406.

Referring to FIG. 30B, finger flange 408 may comprise a first component 412a and a second component 412b. First component 412a may include a first hook 414a and second component 412b may include a second hook 414b. As shown in FIG. 30B, first hook 414a and second hook 414b may abut and/or surround a portion of plunger rod 406. At a pre-injection state, as shown in FIGS. 30A and 30B, finger flange 408 may surround first portion 407a of plunger rod 406. Finger flange 408 may also include a spring (not shown) configured to separate first component 412a and second component 412b (FIG. 30B). Movement of finger flange 408 may be prohibited by the spring and engagement of first hook 414a and second hook 414b with first portion 407a of plunger rod 406. To initiate injection, a user may depress a thumb pad (not shown) plunger rod 406 in a distal direction towards housing 402. During injection, plunger rod 406 may be located in housing 402 such that second portion 407b of plunger rod 406 is received in finger flange 408. In embodiments where second portion 407b may have a diameter that is less than a diameter of first portion 407a, hooks 414a, 414b may no longer abut a portion of plunger rod 406. For example, as shown in FIG. 30D, there may be a space between finger flange 408 may and plunger rod 406, allowing plunger rod 406 to move distally into housing 402 and syringe 404 to dispel contents out of syringe 404. At end-of-dose, as shown in FIG. 30A, the user may maintain depression of the thumb pad, allowing a portion of plunger rod 406 to retreat backwards, e.g., proximally away from housing 402. Spring 410 may then be released, allowing syringe 404 to move proximally towards a distal surface of finger flange 408.

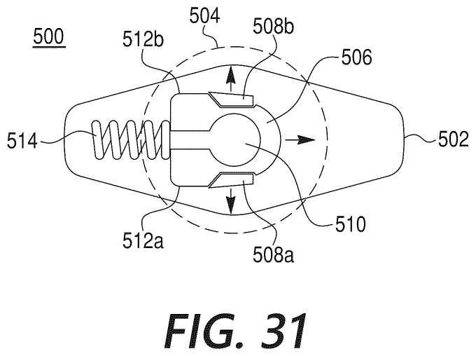

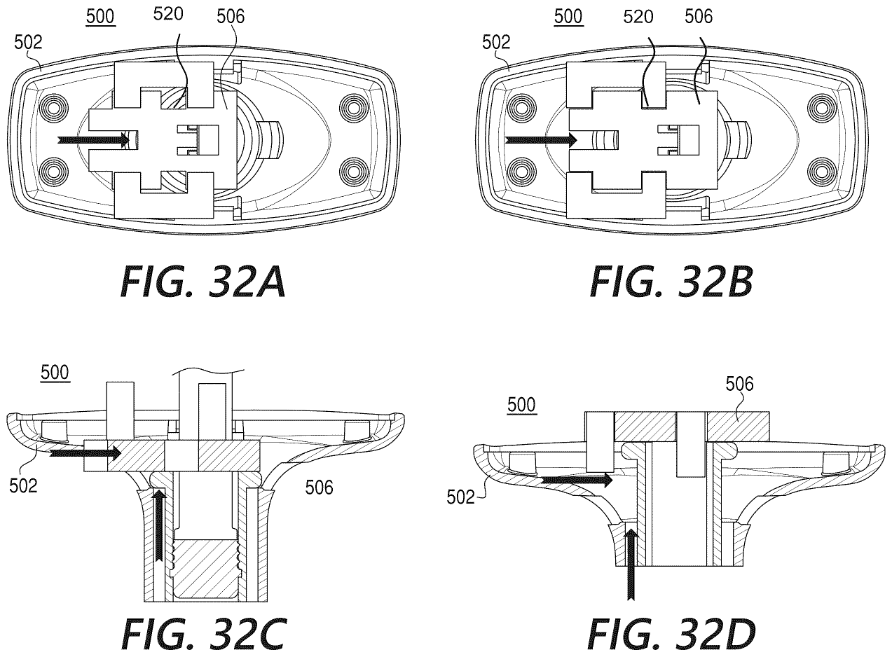

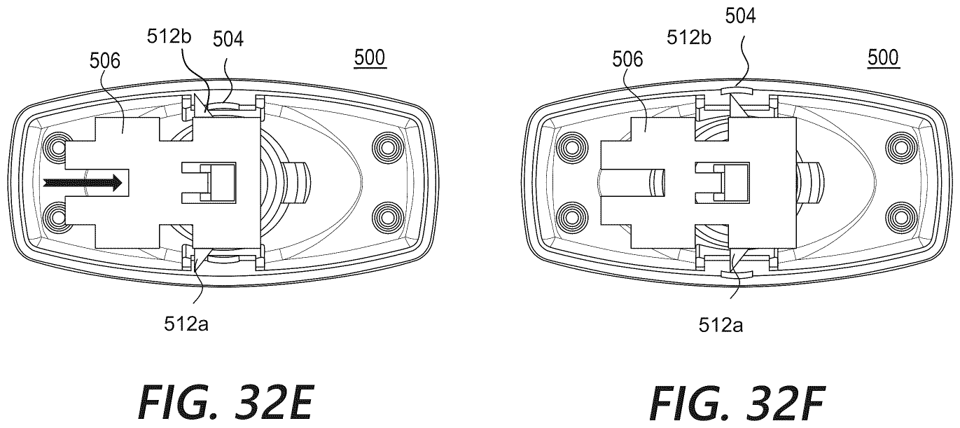

FIGS. 31 and 32A-32F depict another embodiment according to the present disclosure. Features and mechanisms discussed in regards to FIG. 31 and 32a-32F may be utilized and combined with the features of device 400 as described above and shown in FIGS. 30A-3D. FIGS. 31, 32A, 32B, 32E, and 32F depict a top view of a device 500. Device 500 may include a housing (not shown), finger flange 503, a thumb pad 504, and a slider 506. Thumb pad 504 may include a plurality of clips 508a, 508b. Slider 506 may include an opening 510 for receiving a plunger rod (not shown). Clips 508a, 508b may couple thumb pad 504 and a plunger rod in opening 510. A plunger rod utilized in device 500 and with the features of device 500 discussed here may include various geometric features, e.g., recessed portions as shown in FIG. 19B. Referring to FIGS. 31, 32A, 32B, 32E, and 32F, to initiate injection, a user may press the plunger rod in a distal direction. During injection, as a geometry of the plunger rod changes, e.g., in diameter and/or in width, as described above in regards to device 400 in FIGS. 30A-30D, a spring may push slider 506, e.g., in a right direction. As slider 506 is displaced, ramps 512a, 512b of slider 506 may push against clips 508a, 508b. Ramps 512a, 512b may have a width that is larger than a portion of slider 506 that initially sits between clips 508a, 508b. Ramps 512a, 512b may press against clips 508a, 508b, deflecting clips 508a, 508b in an outward direction. This unlocks thumb pad 504 from the plunger rod, while simultaneously allowing a syringe (not shown) to move in a proximal direction and deflecting clips 508a, 508b in an outward direction to lock into the housing, to a lock-out position. FIG. 31 is a top view of device 500 at end-of-dose. FIGS. 32A-32D depict slider 506 including a recess 520. Recess 520 may be shaped as a C-block, e.g., having a C-shape. Recess 520 may interact with portions of the housing to prevent the slider and/or syringe from moving in a proximal direction. These features may eliminate the need for the user to apply extra force at the end of injection to activate a safety mechanism for lockout out the syringe. A spring 514 may be located on the side of slider 506 including the ramps 512a, 512b. Spring 514 may release its stored energy, pushing the syringe in a proximally direction, simultaneously while thumb pad 504 and the plunger rod decouple from each other.

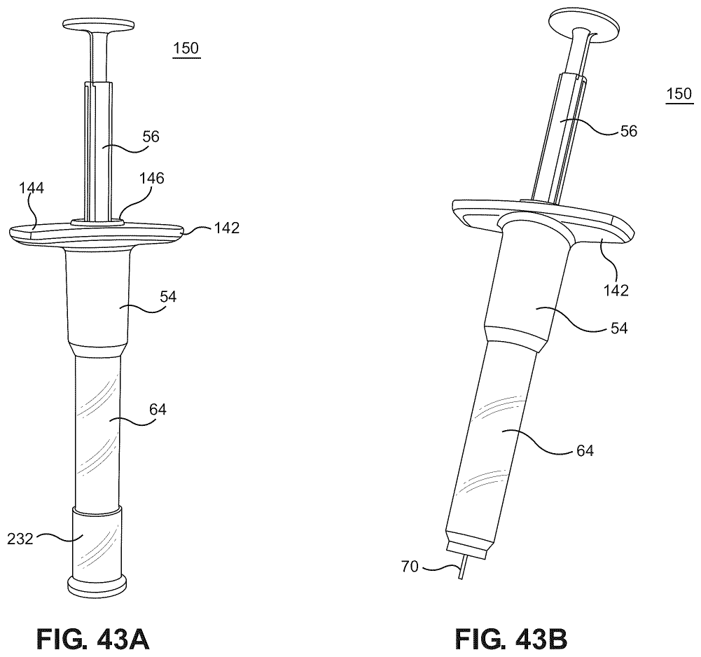

FIGS. 43A-43B depict another embodiment of the present disclosure, similar to those described above in reference to FIGS. 33A-35B. Where appropriate, features will be designated with references similar to those discussed above. Any features and components that correspond to those in FIGS. 33A-35B may be understood to be configured similarly. FIG. 43A shows a perspective view of the exterior of device 150, and FIG. 43B shows a perspective view of the exterior of device 150 without a needle cap 232. Device 150 may include plunger rod 56, housing 54, and needle cap 232, among other components. Housing 54 may include a finger flange 142 and rear cap 144. Rear cap 144 may also be referred to here as a blocking component. Finger flange 142 may have any appropriate size and/or configuration allowing for proper use. A user may utilize one or two hands to operate device 150. For example, one hand of the user may hold device 150, while the other hand of the user pushes plunger rod 56 in a distal direction towards finger flange 142. In other examples, the user may utilize one hand to both hold device 150 and push plunger rod 56 in a distal direction towards finger flange 142. As shown in FIG. 43A, rear cap 144 may include a collar 146. Collar 146 may extend in a proximal direction away from finger flange 142 and contact a proximal surface of thumb pad 492 as the injection dose is completed. Collar 146 may be any appropriate size and/or configuration to surround a portion of plunger rod 56. In some embodiments, collar 146 may have a generally circular shape.

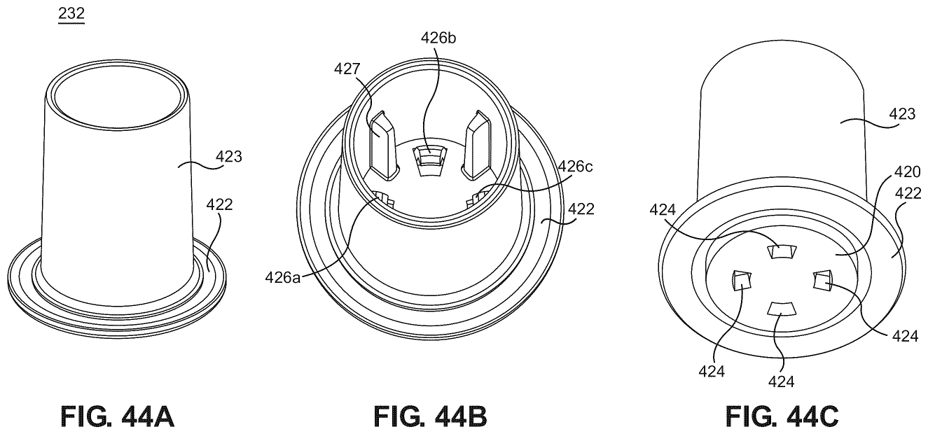

FIGS. 44A-44C depict needle cap 232, similar to the embodiment as described above with reference to FIGS. 40A-40C. Needle cap 232 may be attached to a distal end of device 150, as shown in FIG. 43A. In some examples, needle cap 232 may include a cap neck 423 and a cap skirt 422. As shown in FIGS. 43A and 44C, cap skirt 422 may surround a distal end 420 of needle cap 232. Distal end 420 may include a plurality of openings 424. For example, distal end 420 may include at least one opening 424, at least two openings 424, at least three openings 424, at least four openings 424, or more than four openings 424. Openings 424 may serve as anti-choke holes. In some examples, during manufacturing, when there are four openings 424, openings 424 may be made to create clips 426a, 426b, 426c, 426d, not shown. In some examples, needle cap 232 may include a plurality of clips 426a, 426b, 426c, 426d. In some examples, needle cap 232 may include at least one clip, at least two clips, at least three clips, or at least four clips. Each clip 426a, 426b, 426c, 426d may be shaped and/or configured to attach to, e.g., latch onto, a corresponding ledge 428a, 428b, 428c, 428d of needle shield 430, as shown in FIGS. 41A-41B. For example, clip 426a may latch onto ledge 428a, clip 426b may latch onto ledge 428b, clip 426c may latch onto ledge 428c, and clip 426d may latch onto ledge 428d. In some examples, needle cap 232 may include two, three, four, or more spines 427. Spines 427 may be configured to restrict proximal movement of device 150 into needle cap 232. Spines 427 may be deflectable and/or deformable. Spines 427 may be rigid.

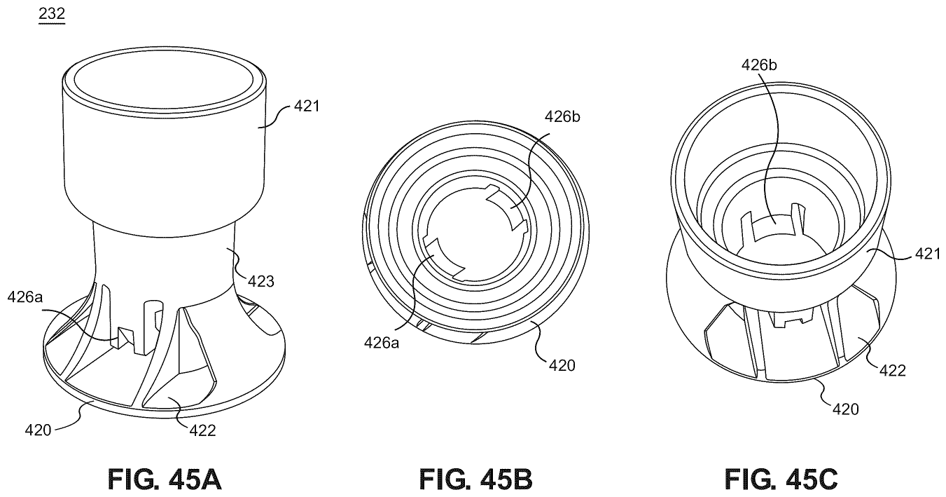

FIGS. 45A-45C depict needle cap 232, similar to needle cap 232 as described above with reference to FIGS. 40A-40C and 44A-44C. Needle cap 232 may be attached to a distal end of device 150. In some examples, needle cap 232 may include a cap collar 421, a cap neck 423, and a cap skirt 422. As shown in FIGS. 43A and 44C, cap skirt 422 may surround a distal end 420 of needle cap 232. In some examples, needle cap 232 may include a plurality of clips 426a, 426b. In some examples, needle cap 232 may include at least one clip or at least two clips. Each clip 426a, 426b may be shaped and/or configured to attach to, e.g., latch onto, a corresponding ledge 428a, 428b of needle shield 430, as shown in FIGS. 41A-41B. For example, clip 426a may latch onto ledge 428a and clip 426b may latch onto ledge 428b, as shown in FIGS. 41A-41B.

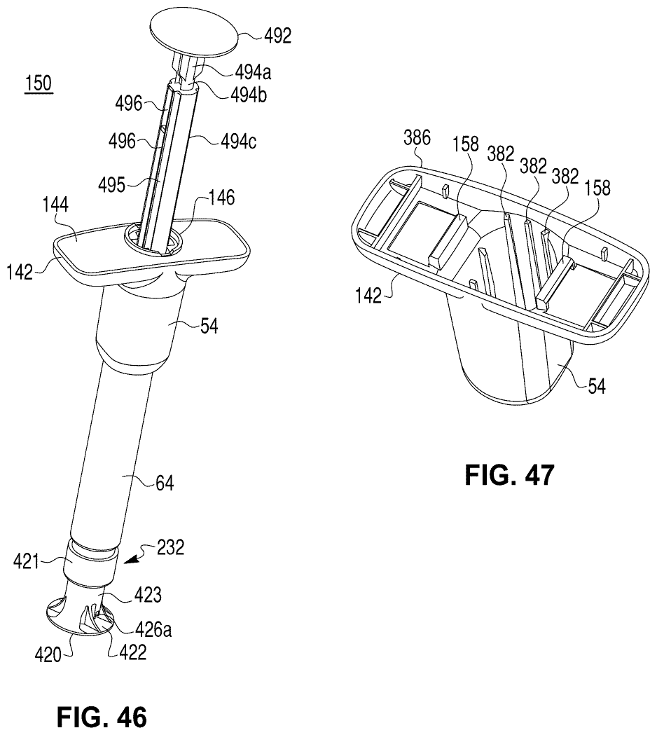

FIG. 46 shows a perspective view of the exterior of device 150 without a needle cap 232, similar to the embodiment as described above with reference to FIG. 34. Device 150 may include plunger rod 56, housing 54, needle cover 64, and needle cap 232. Housing 54 may include a finger flange 142 having recesses 384. Finger flange 142 may have any appropriate size and/or configuration allowing for proper use. Plunger rod 56 may include a thumb pad 492 for user to depress plunger rod 56 in a distal direction. A user may utilize one or two hands to operate device 150. For example, one hand of the user may hold device 150, while the other hand of the user pushes thumb pad 492 of plunger rod 56 in a distal direction towards finger flange 142. In other examples, the user may utilize one hand to both hold device 150 and push plunger rod 56 in a distal direction towards finger flange 142. In some embodiments, and as discussed above in reference to FIGS. 42A-42D, plunger rod 56 may include a first portion 494a having a first diameter, a second portion 494b having a second diameter, and a third portion 494c having a third diameter. The first diameter of first portion 494a and the third diameter of third portion 494c may each be greater than the second diameter of second portion 494b. Portions of plunger rod 56 may be cored out to form a plurality of ribs 495 and/or grooves 496. Needle cap 232 may cover needle 70, as shown in FIG. 43B, and may include a cap collar 421, a cap neck 423, and a cap skirt 422. Needle cap 323 may include at least one clip 426a may be shaped and/or configured to attach to, e.g., latch onto, a corresponding ledge 428a, of needle shield 430, as shown in FIGS. 41A-41B.

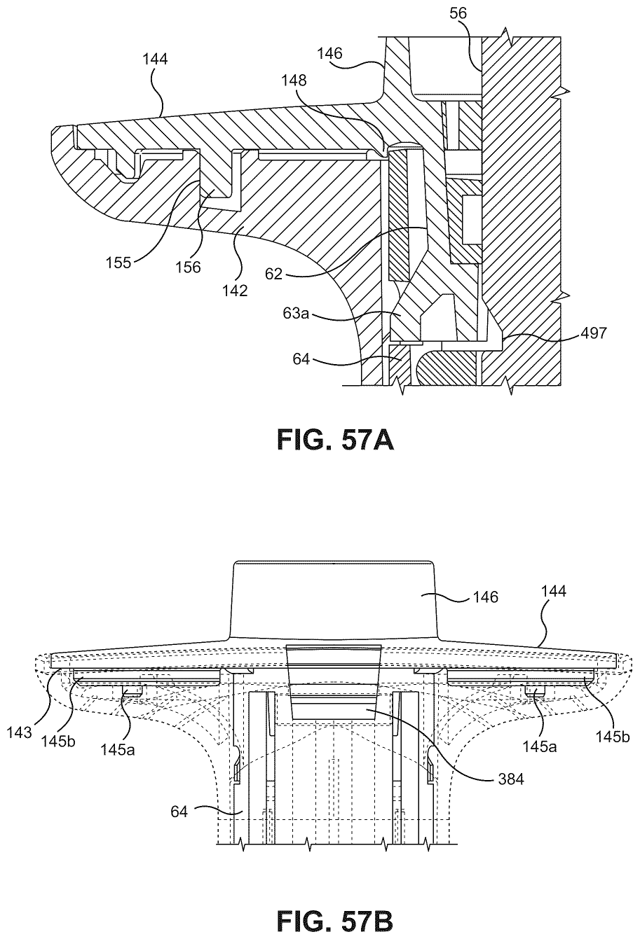

FIG. 47 depicts details of housing 54. Housing 54 may include finger flange 142 as described in detail throughout the present disclosure. In some examples, housing 54 may include a plurality ribs 382. Each rib of plurality of ribs 382 may each have any appropriate size, shape, and/or configuration to maintain a position of syringe 52 within housing 54. For example, each rib of plurality of ribs 382 may have a generally rectangular shape to maintain a position of syringe 52 within housing 54. Each rib of plurality of ribs 382 may have a generally trapezoidal to guide into and to maintain a position of syringe 52 within housing 54. Housing 54 may include a plurality of ledge portions 158 configured to receive rear cap hook 149, as shown in FIGS. 57A-57B Ledge portions 158 and rim 386 may each have any appropriate size, shape, and/or configuration, such that rear cap hook 149 may securely couple and/or permanently attach to housing 54.

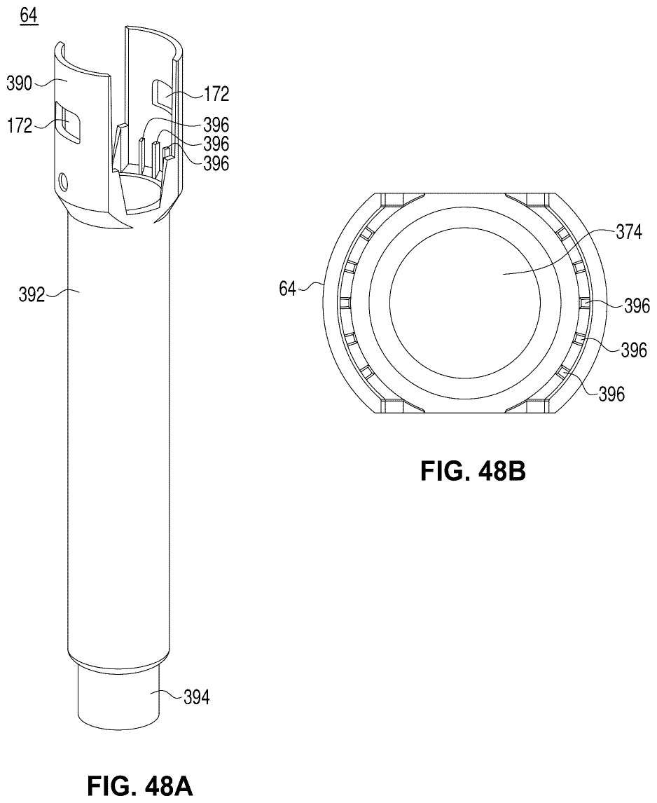

FIGS. 48A-48B depict details of needle cover 64. Needle cover 64 may include at least one opening 172, as described above in regards to FIGS. 36A-36D. As shown in FIG. 48A, needle cover 64 may include at least two openings 172. Opening 172 may have any appropriate size, shape, and/or configuration, for receiving a portion of first pair of deflectable arms 63a of blocking component 62, as shown in FIGS. 15B-15B. In some examples, opening 172 may have a generally rectangular shape, but other suitable shapes are contemplated. In some examples, needle cover 64 may include a proximal portion 390, a shaft 392, and a neck 394. The diameters of needle cover 64 may vary at the different sections. For example, referring to FIG. 48A, an external diameter of proximal portion 390 may be larger than each of an external diameter of shaft 392 and/or an external diameter of neck 394. In some examples, the external diameter of shaft 392 may be less than the external diameter of proximal portion 390, but greater than the external diameter of neck 394. The internal diameters may also vary at the different sections. As described above with respect to FIGS. 39B-39C, needle cover 64 may also include at least one protrusion 396. As shown in FIG. 48B, needle cover 64 may include at least one protrusion 396 located on an interior surface of needle cover 64 with the length of the protrusion extending in the same direction as the length of needle cover 64. In some examples, needle cover 64 may include a plurality of protrusions 396. For example, needle cover 64 may include at least one protrusion 396, at least two protrusions 396, at least three protrusions 396, or at least four protrusions 396. Protrusions 396 may have any appropriate size, shape, and/or configuration to secure syringe 52. For example, protrusions 396 may be circumferentially spaced apart, where each of the protrusions 396 extends in the same orientation. As shown in FIG. 48B, needle cover 64 forms a periphery surrounding an interior cavity 374 configured to receive syringe 52.

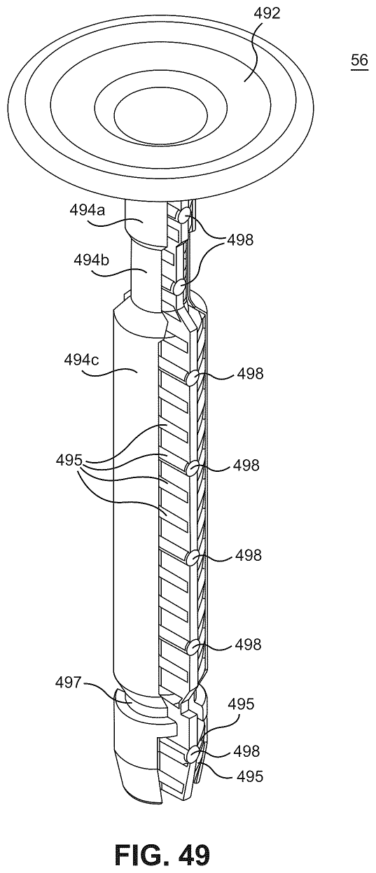

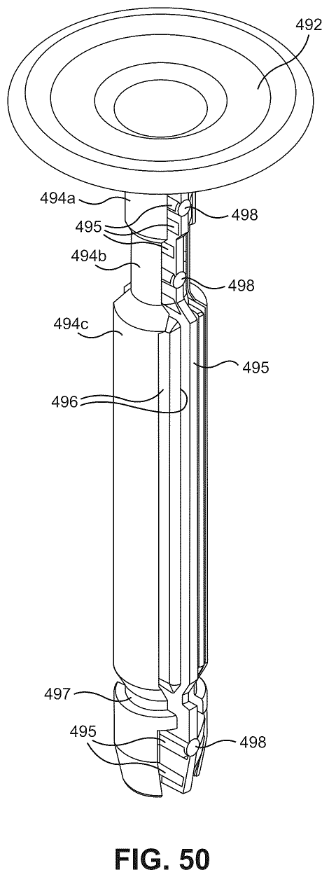

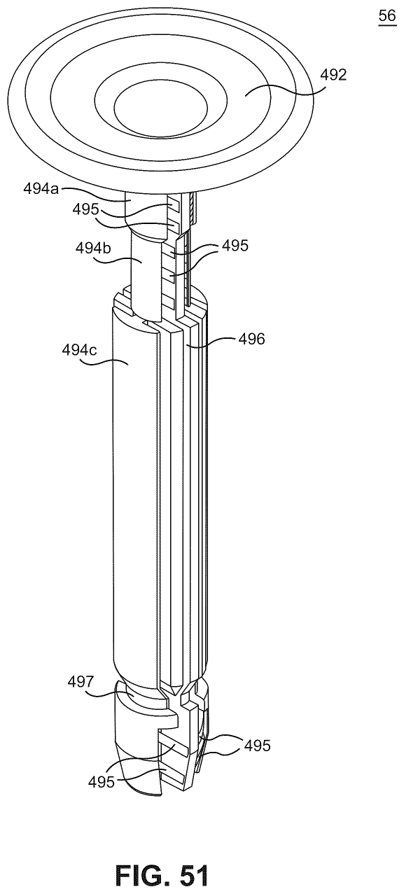

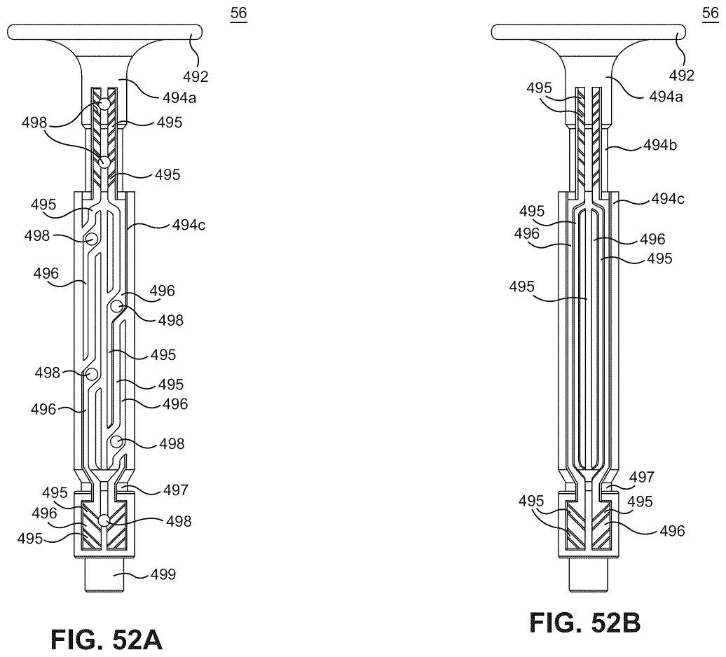

FIG. 49-52B depict alternative embodiments of plunger rod 56. As shown above in reference to FIG. 36A, plunger rod 56 may include a thumb pad 492 for user to depress plunger rod 56 in a distal direction. In some embodiments, and as discussed above in reference to FIGS. 35A-35B and FIG. 46, plunger rod 56 may include a first portion 494a having a first diameter, a second portion 494b having a second diameter, and a third portion 494c having a third diameter. The first diameter of first portion 494a and the third diameter of third portion 494c may each be greater than the second diameter of second portion 494b. Plunger rod 56 may include indent 497, which may correspond to indent 170 as described above in reference to FIG. 36B. Plunger rod 56 may have various configurations, for example, as shown in FIGS. 49-52B. For example, portions of plunger rod 56 may be cored out to form a plurality of ribs 495 and/or grooves 496. As shown in FIGS. 52A and 52B, the plurality of ribs 495 and/or grooves 496 may be symmetrical or asymmetrical As shown in FIG. 51-52B, the plurality of ribs 495 may longitudinally extend along a length of plunger rod 56 and/or laterally extend along a width of plunger rod 56. In some embodiments, the plurality of ribs 495 may longitudinally extend along a length of third portion 494c of plunger rod 56. The plurality of ribs 495 and/or grooves 496 may form locations for placement of ejector pins 498. The placement of ejector pins 498 may affect the injection force as plunger rod 56 slides past components of the devices discussed herein, e.g., cap 144. To decrease the injection force, ejector pins 498 may be placed in areas of the device other than the external surface of plunger rod 56. For example, ejector pins 498 may be placed in the internal surface of plunger rod 56. Grooves 496 may have any appropriate size, shape, and/or configuration to correspond with the plurality of ribs 382 to maintain a position of syringe 52 within housing 54. The distal end of plunger rod 56 may further include a spike 499. Spike 499 may have any appropriate size, shape, and/or configuration to advance within needle cover 64.

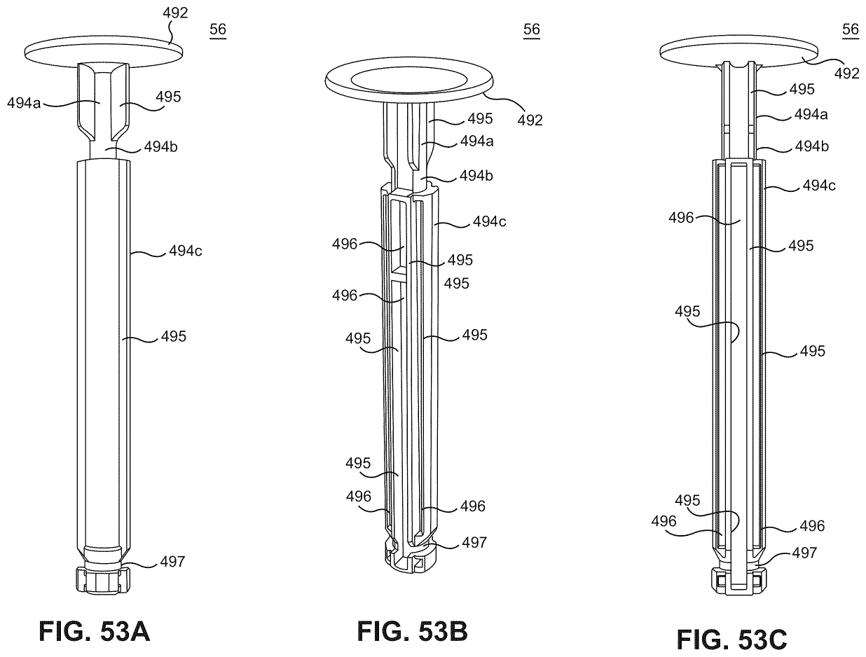

FIG. 53A-53C depict alternative embodiments of plunger rod 56. As shown in FIG. 53A, the plurality of ribs 495 may longitudinally extend along a length of plunger rod 56 and/or laterally extend along a width of plunger rod 56. As shown in FIGS. 53B-53C, plunger rod 56 may be include an indent 497, which may correspond to indent 170 as described above in reference to FIG. 36B, and may be slidably coupled to blocking component 62, such that plunger rod 56 may slide in both a distal and proximal direction. FIG. 57A depicts a cross-sectional view of a quarter portion of an interior of device 150. Device 150 may include needle cover 64, plunger rod 56, finger flange 142, and rear cap 144. Device 150 may include various features to couple finger flange 142 and rear cap 144 together. For example, finger flange 142 may include a snap 155 and rear cap 144 may include an insert 156. Snap 155 and insert 156 may engage, e.g., snap together, to couple rear cap 144 to finger flange 142. Rear cap 144 may further include a ridge 148. Ridge 148 may extend from a bottom side of rear cap 144 to prevent deflection of rear cap 144 when plunger rod 56 is in use. Plunger rod 56 may be include an indent 497 which may correspond to indent 170 as described above in reference to FIG. 15B, and may be slidably coupled to blocking component 62, such that plunger rod 56 may slide in both a distal and proximal direction. Blocking component 62 may include deflectable arm 63 extending in a distal direction. Blocking component 62 may include more than one deflectable arm 63. For example, blocking component 62 may include two, three, four, or more deflectable arms 63. In configurations with more than one deflectable arm 63, the deflectable arms 63 may be equally spaced apart from one another. For example, if the blocking component 62 includes four deflectable arms 63, each of the deflectable arms 63 may be about 90 degrees from one another. Indent 497 may be configured to receive deflectable arm 63.

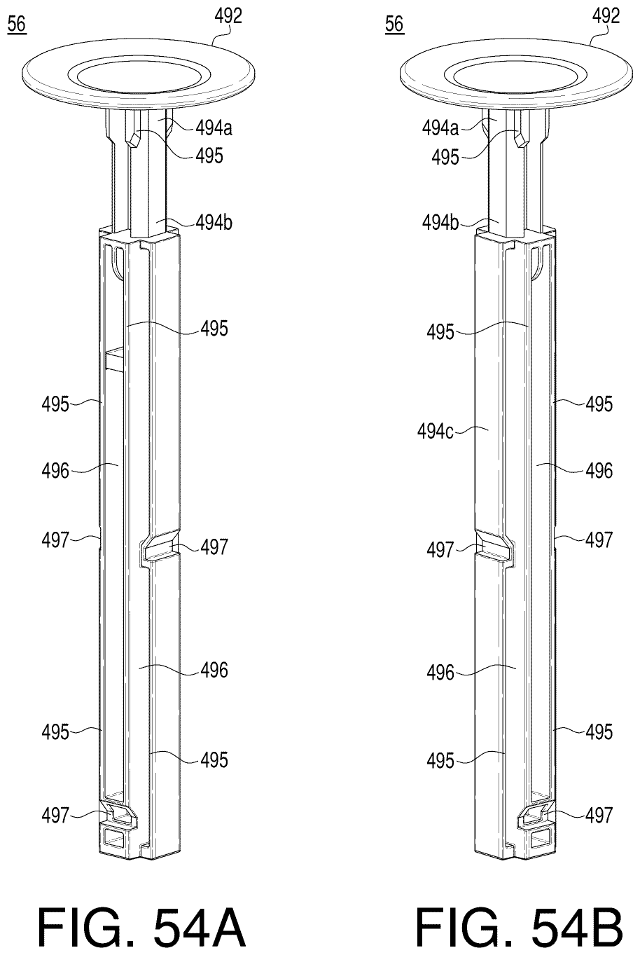

FIGS. 54A-54B depict an alternative embodiment of plunger rod 56. Plunger rod 56 may be used in any of the devices shown in the figures or described herein, such as device 140, device 150, or another device. As FIGS. 54A-54B show, plunger rod 56 may include multiple indents 497, such as multiple sets of indents 497. For example, FIGS. 54A-54B show two sets of two indents 497, and thus plunger rod 56 may include four indents 497. In some embodiments, plunger rod 56 may include at least two sets of indents 497, at least three set of indents 497, or at least four sets of indents. In other embodiments, plunger rod 56 may include at least two indents 497, at least three indents 497, at least four indents 497, at least five indents 497, at least six indents 497, at least seven indents 497, or at least eight indents 497. Indents 497 may be positioned at different locations, e.g., at different heights along plunger rod 56, and/or on different faces, sides, or surfaces of plunger rod 56. As FIGS. 54A-54B show, two indents 497 in a first set may be on opposite sides, surfaces, or faces of plunger rod 56 from one another, and may be at one height along plunger rod 56; and two indents 497 in a second set may be on opposite sides, surface, or faces of plunger rod 56 from one another, and may be at another height along plunger rod 56. Further, as FIGS. 54A-54B show, indents 497 in one set may be on different sides, surfaces, or faces of plunger rod 56 than indents 497 in another set, such that the sets of indents 497 are disposed at about 90 degrees offset from one another.

The locations of indents 497 may correspond to different injection doses to be delivered by the device in which plunger rod 56 is installed. Thus, for example, when plunger rod 56 is installed in device 150, and a size of the injection dose for delivery by device 150 is a relatively larger dose of two doses, plunger rod 56 may be installed in device 150 such that two deflectable arms 63 are received in the set of two indents 497 which is farther from thumb pad 492, i.e., closer to a proximal end of plunger rod 56. By way of further example, when a size of the injection dose for delivery by device 150 is a relatively smaller dose of two doses, plunger rod 56 may be installed in device 150 such that two deflectable arms 63 are received in the set of two indents 497 which is closer to thumb pad 492, i.e., closer to a distal end of plunger rod 56. In some instances, the larger dose of the two doses may be about 2.0 mL, about 2.5 mL, about 3.0 mL, about 3.3 mL, or about 3.5 mL. In some instances, the larger dose of the two doses may be between about 2.0 mL to about 3.5 mL, about 2.5 mL to about 3.5 mL, about 3.0 mL to about 3.5 mL, about 3.3 mL to about 3.5 mL, about 2.0 mL to about 3.3 mL, about 2.5 mL to about 3.3 mL, about 3.0 mL to about 3.3 mL, about 2.0 mL to about 3.0 mL, about 2.5 mL to about 3.0 mL, or about 2.0 mL to about 2.5 mL. In some instances, the smaller dose of the two doses may be about 1.0 mL, about 1.5 mL, about 2.0 mL, about 2.5 mL, or about 3.0 mL. In some instances, the smaller dose of the two doses may be between about 1.0 mL to about 3.0 mL, about 1.5 mL to about 3.0 mL, about 2.0 mL to about 3.0 mL, about 1.0 mL to about 2.5 mL, about 1.5 mL to about 2.5 mL, about 2.0 mL to about 2.5 mL, about 1.0 mL to about 2.0 mL, about 1.5 mL to about 2.0 mL, or about 1.0 mL to about 1.5 mL.

Although FIGS. 54A-54B illustrate the sets of indents 497 in a specific configuration, the sets of indents 497 may be positioned differently than FIGS. 54A-54B show. For example, the set of two indents 497 farther from thumb pad 492 and the set of two indents 497 closer to thumb pad 492 may be switched with one another. By way of another example, the set of two indents 497 farther from thumb pad 492 may be disposed at a different position along plunger rod 56—either closer to or farther from thumb pad 492. By way of still another example, the set of two indents 497 closer to thumb pad 492 may be disposed at a different position along plunger rod 56—either closer to or farther from thumb pad 492.

As discussed, a device in which plunger rod 56 is installed may include one or more deflectable arms (e.g., deflectable arms 63 of device 50, deflectable arms 83 of device 80, deflectable arms 63a or 63b of device 140 or 150, or deflectable arms 302 of device 290), and may include indents 497 configured to receive the deflectable arms. Thus, as described, for example, the device may include two deflectable arms that are offset about 180 degrees from one another. Therefore, depending on the orientation of plunger rod 56 when installed in the device, one set of two indents 497 may be positioned to receive the two deflectable arms, while the other set of two indents 497 may be positioned so as not to receive the deflectable arms. Accordingly, depending on the orientation of plunger rod 56, plunger rod 56 may deliver a relatively larger or a relatively smaller dose.

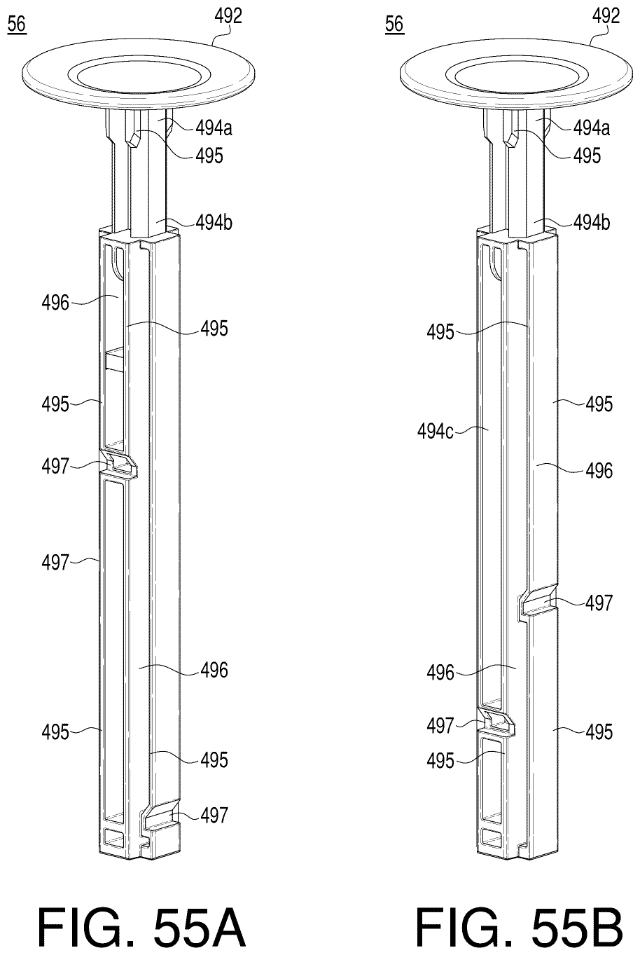

FIGS. 55A-55B depict an alternative embodiment of plunger rod 56. Plunger rod 56 includes four individual indents 497, each of which is on a separate face, side, or surface of plunger rod 56. The embodiment of plunger rod 56 may be installed in a device that may include one deflectable arm, as described above. Thus, depending on the orientation of plunger rod 56 when installed in the device, one of the four indents 497 may be positioned to receive the deflectable arm, while the other three indents 497 may be positioned so as not to receive the deflectable arm. Accordingly, depending on the orientation of plunger rod 56 within the device, plunger rod 56 may deliver one of four corresponding doses. Although FIGS. 55A-55B show plunger rod 56 with the four indents 497 on specific sides, surface, or faces, and at specific heights along plunger rod 56, the individual indents 497 may be on other sides, surface, or faces of plunger rod 56 than FIGS. 55A-55B show, or may be at different heights along plunger rod 56.

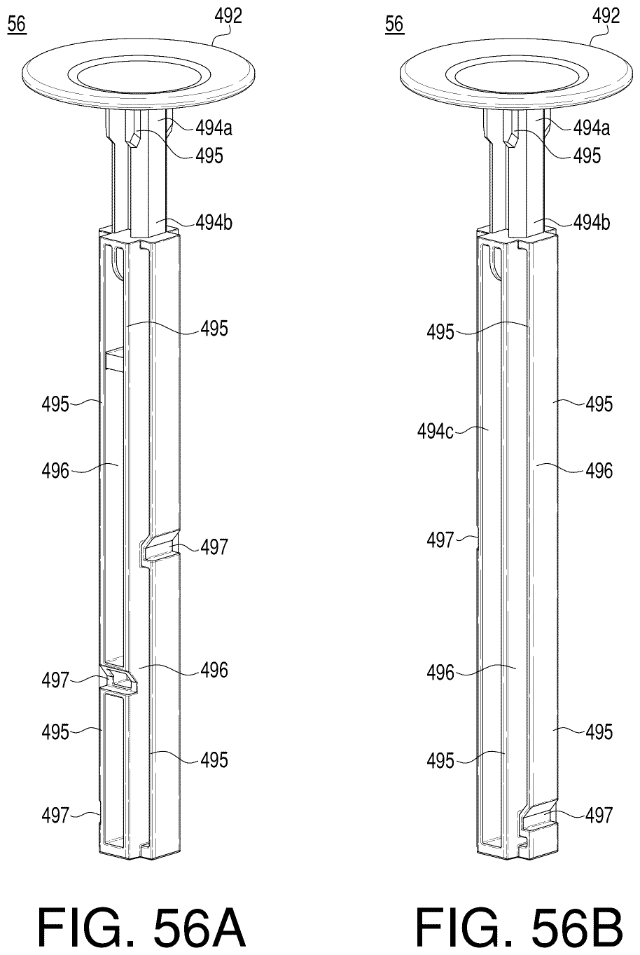

FIGS. 56A-56B depict an alternative embodiment of plunger rod 56. Plunger rod 56 includes three individual indents 497, each of which is on a separate face, side, or surface of plunger rod 56. The embodiment of plunger rod 56 may be installed in a device that may include one deflectable arm, as described. Thus, depending on the orientation of plunger rod 56 when installed in the device, one of the three indents 497 may be positioned to receive the deflectable arm, while the other two indents 497 may be positioned so as not to receive the deflectable arm. Accordingly, depending on the orientation of plunger rod 56 within the device, plunger rod 56 may deliver one of three corresponding doses. Although FIGS. 56A-56B show plunger rod 56 with the three indents 497 on specific sides, surface, or faces, and at specific heights along plunger rod 56, the individual indents 497 may be on other sides, surface, or faces of plunger rod 56 than FIGS. 56A-56B show, or may be at different heights along plunger rod 56.

In other embodiments, plunger rod 56 may include a greater or fewer number of indents 497, on different sides, surfaces, or faces of plunger rod 56, at different locations on plunger rod 56, such that plunger rod 56 may be configured to provide different dose sizes (such as the different injection sizes described herein above) depending on the orientation of plunger rod 56 within the device (e.g., depending on which of the indents 497 are positioned to receive the deflectable arms of the device).

FIG. 57B depicts alternative embodiments of rear cap 144. Rear cap 144 may include a collar 146. Collar 146 may extend in a proximal direction from a proximal side of 144 and contact a proximal surface of thumb pad 492 as the injection dose is completed. In some examples, rear cap 144 may include a plurality of protrusions 145a, 145b which may extend in a distal direction away from distal surface 143. Plurality of protrusions 145a, 145b may have any appropriate size, shape, and/or configuration, such that plurality of protrusions 145a, 145b may securely couple and/or permanently attach to housing 54. Rear cap 144 may include a plurality of recesses 384 to correspond to needle cover 64.

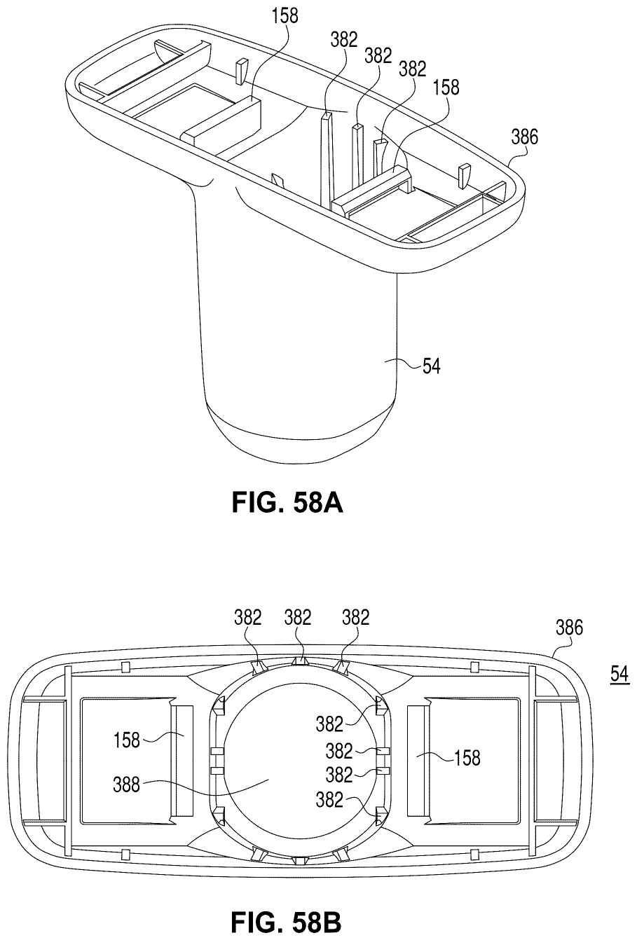

FIGS. 58A-58B depict details of housing 54, as described above in regards to FIG. 47. Housing 54 may include finger flange 142 as described in detail throughout the present disclosure and opening 388 configured to receive syringe 52. Opening 388 may have a generally circular shape. In some examples, housing 54 may include a plurality ribs 382. Each rib of plurality of ribs 382 may have any appropriate size, shape, and/or configuration to maintain a position of syringe 52 within housing 54

Each rib of plurality of ribs 382 may have a generally rectangular shape to maintain a position of syringe 52 within housing 54. Plurality of ribs 382 may be arranged symmetrically or asymmetrically with respect to opening 388. Housing 54 may include a plurality of ledge portions 158 configured to receive rear cap hook 149, as shown in FIGS. 37C-37D. Ledge portions 158and rim 386 may each have any appropriate size, shape, and/or configuration, such that rear cap hook 149 may securely couple and/or permanently attach to housing 54.

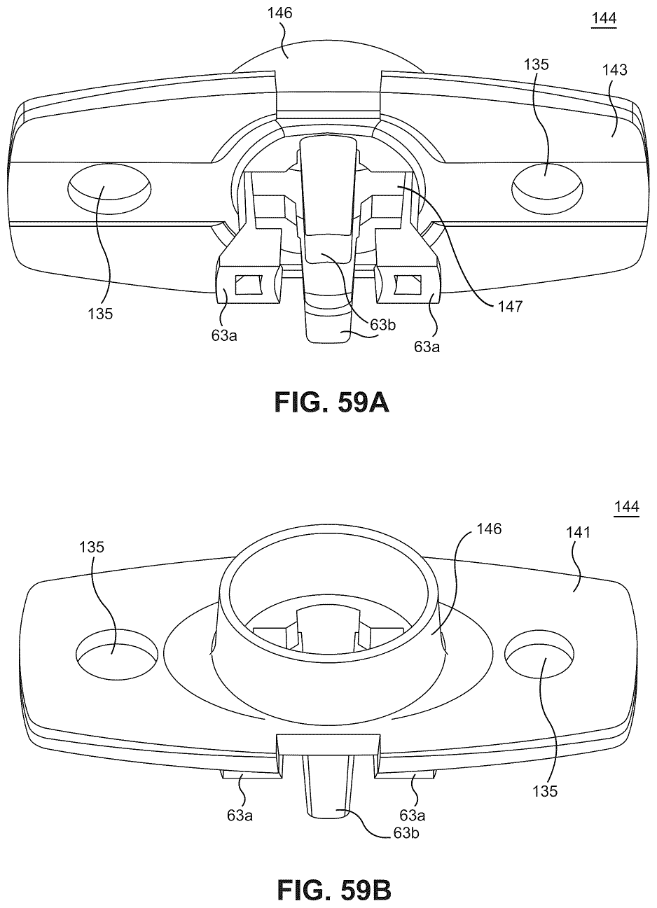

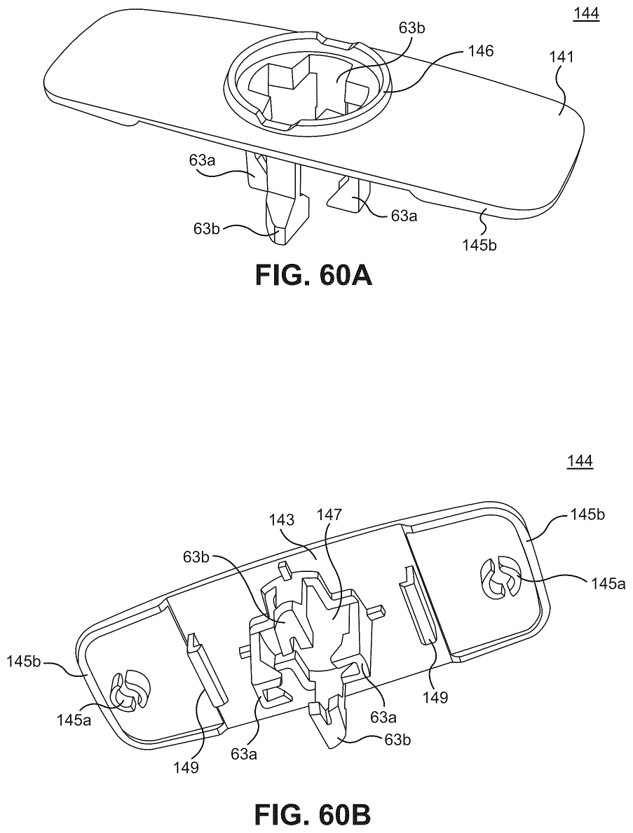

FIGS. 59A-60B depict details of rear cap 144, similar to those described above in reference to FIGS. 14A-16B and FIGS. 37A-37B. As shown in FIG. 59A-59B, collar 146 may surround opening 147. Opening 147 may be configured to receive a portion of plunger rod 56. In some examples, opening 147 may have a generally circular shape. As shown in FIG. 60A, opening 147 may have a cross shape. In some examples, rear cap 144 may include holes 135 extending from proximal surface 141 to distal surface 143. In some examples, rear cap 144 may include a plurality of protrusions 145a, 145b, which may extend in a distal direction away from distal surface 143. Plurality of protrusions 145a, 145b may have any appropriate size, shape, and/or configuration, such that plurality of protrusions 145a, 145b may securely couple and/or permanently attach to housing 54. For example, first plurality of protrusions 145a may have a generally triangular, circular, semi-circular, rectangular, or cuboidal shape. The shape of first plurality of protrusions 145a may correspond to recesses 380 (FIGS. 58A and 58B) in housing 54. In other examples, second plurality of protrusions 145b may fit into and/or abut a corresponding rim 386 of housing 54.

First pair of deflectable arms 63a and second pair of deflectable arms 63b are described in detail above, with respect to FIGS. 37A-37D. FIGS. 59A-60B depict exemplary configurations of first pair of deflectable arms 63a and second pair of deflectable arms 63b. For example, a first arm of the first pair of deflectable arms 63a may be located opposite from a second arm of the first pair of deflectable arms 63a. Further, a first arm of the second pair of deflectable arms 63b may be located opposite from a second arm of the second pair of deflectable arms 63b. In embodiments where there are four deflectable arms, each deflectable arm may be about 90 degrees from one another. In some embodiments, an arm of the first pair of deflectable arms 63a may alternate with an arm of the second pair of deflectable arms 63b about the circumference of opening 147. As shown in FIG. 60B, rear cap 144 may include a rear cap hook 149 for abutting against a ledge portion 158 (FIG. 35B) of housing 54. In some examples, rear cap 144 may include a plurality of rear cap hooks 149 for abutting against a plurality of ledge portions 158 of housing 54. For example, rear cap 144 may include at least two rear cap hooks 149, at least three rear cap hooks 149, or at least four rear cap hooks 149.

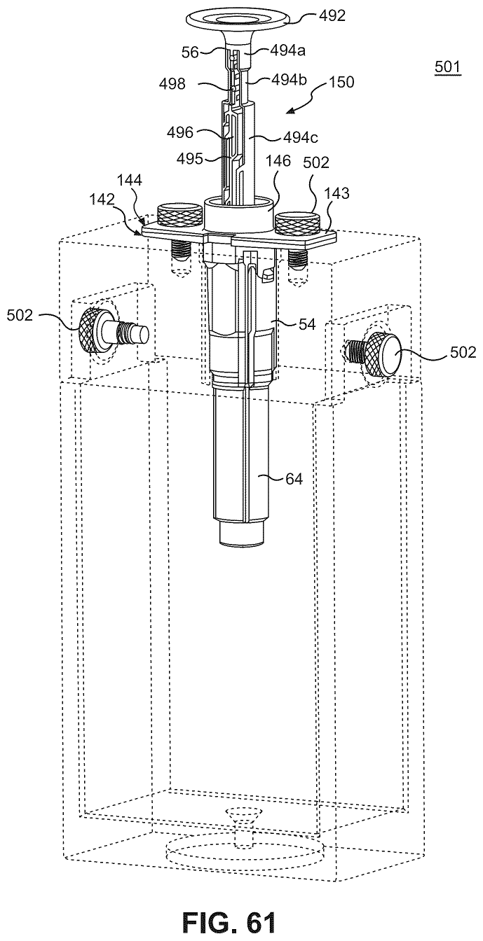

FIG. 61 depicts device 150 in a test system 501. Device 150 may include plunger rod 56, housing 54, and needle cover 64. Housing 54 may include finger flange 142 configured to be coupled to rear cap 144. Rear cap 144 may include collar 146. Collar 146 may extend in a proximal direction away from finger flange 142. Collar 146 may be any appropriate size and/or configuration to surround a portion of plunger rod 56. In some embodiments, collar 146 may have a generally circular shape. Portions of plunger rod 56 may be cored out to form a plurality of ribs 495 and/or grooves 496. The plurality of ribs 495 and/or grooves 496 may form locations for placement of ejector pins 498. In some embodiments, rear cap 144 may include holes 135 extending from proximal surface 141 to distal surface 143. Holes 135 may extend from a distal side of finger flange 142 to proximal surface of 141 when rear cap 144 and finger flange 142 are coupled together. Screw 502 may extend through hole 135. Holes 135 may be any appropriate size and/or configuration to surround a portion of screw 502. Screw 502 may secure device 150 in test system 501. Test system 501 may test the functionality of device 150, such as the force required to depress plunger 56 and/or the dose delivered. Although FIG. 61 illustrates test system 501 testing device 150, testing system 501 may be used to test any of the described or illustrated devices, with any of the disclosed or illustrated embodiments of plunger rod 56.

The embodiments described herein may be used in connection with the administration of various medicaments, drugs, and/or pharmaceutical formulations to patients. Exemplary medicaments, drugs, and/or pharmaceutical formulations with which the embodiments described herein may be used are described in U.S. Pat. Nos. 8,945,559B2, 9,987,500 B2, and 11,603,407 B2, the entireties of which are incorporated herein by reference. Medicaments, drugs, and/or pharmaceutical formulations that may be used with the embodiments of the present disclosure are described in further detail hereinafter.

First, exemplary medicaments, drugs, and/or pharmaceutical formulations consistent with U.S. Pat. No. 8,945,559 B2 are described. As used herein, the expression “pharmaceutical formulation” means a combination of at least one active ingredient (e.g., a small molecule, macromolecule, compound, etc. which is capable of exerting a biological effect in a human or non-human animal), and at least one inactive ingredient which, when combined with the active ingredient or one or more additional inactive ingredients, is suitable for therapeutic administration to a human or non-human animal. The term “formulation”, as used herein, means “pharmaceutical formulation” unless specifically indicated otherwise.

The present disclosure provides pharmaceutical formulations comprising at least one therapeutic polypeptide. According to certain embodiments of the present disclosure, the therapeutic polypeptide is an antibody, or an antigen-binding fragment thereof, which binds specifically to human interleukin-4 receptor alpha (hIL-4Rα). More specifically, the present disclosure includes pharmaceutical formulations that comprise: (i) a human antibody that specifically binds to hIL-4Rα; (ii) an acetate/histidine buffer system; (iii) an organic cosolvent that is a non-ionic surfactant; (iv) thermal stabilizer that is a carbohydrate; and (v) a viscosity reducer. Specific exemplary components and formulations included within the present disclosure are described in detail below.

The pharmaceutical formulations of the present disclosure may comprise a human antibody, or an antigen-binding fragment thereof, that binds specifically to hIL-4Rα. As used herein, the term “hIL-4Rα” means a human cytokine receptor that specifically binds interleukin-4 (IL-4). In certain embodiments, the antibody contained within the pharmaceutical formulations of the present disclosure binds specifically to the extracellular domain of hIL-4Rα. An exemplary human IL-4 receptor alpha (hIL-4Rα) amino acid sequence is described in SEQ ID NO:25. Antibodies to hIL- 4Rα are described in U.S. Pat. Nos. 7,605,237 and 7,608,693. The extracellular domain of hIL-4Rα is represented by the amino acid sequence of SEQ ID NO: 26.

The term “antibody”, as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (e.g., lacking light chains) are also encompassed within the definition of the term “antibody”. Each heavy chain comprises a heavy chain variable region (abbreviated herein as HCVR or V_{H}) and a heavy chain constant region. The heavy chain constant region comprises three domains, CH1, CH2 and CH3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementary determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

Unless specifically indicated otherwise, the term “antibody”, as used herein, shall be understood to encompass complete antibody molecules as well as antigen-binding fragments thereof. The term “antigen-binding portion” or “antigen-binding fragment” of an antibody (or simply “antibody portion” or “antibody fragment”), as used herein, refers to one or more fragments of an antibody that retain the ability to specifically bind to hIL-4Rα or an epitope thereof.

An “isolated antibody”, as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Rα).

The term “specifically binds”, or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4R molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material or chemicals.

Exemplary anti-hIL-4Rα antibodies that may be included in the pharmaceutical formulations of the present disclosure are set forth in U.S. Pat. Nos. 7,605,237 and 7,608,693, the disclosures of which are incorporated by reference in their entirety.

According to certain embodiments of the present disclosure, the anti-hIL-4Rα antibody is a human IgG1 comprising a heavy chain variable region that is of the IGHV3-9 subtype and a light chain variable region that is of the IGKV2-28 subtype (see Barbie and Lefranc, The Human Immunoglobulin Kappa Variable (IGKV) Genes and Joining (IGKJ) Segments, Exp. Clin. Immunogenet. 1998; 15:171-183; and Scaviner, D. et al., Protein Displays of the Human Immunoglobulin Heavy, Kappa and Lambda Variable and Joining Regions, Exp. Clin. Immunogenet., 1999; 16:234-240).

In some embodiments, the anti-hIL-4Rα comprises at least one amino acid substitution, which results in a charge change at an exposed surface of the antibody relative to the germline IGHV3-9 sequence or the germline IGKV2-28 sequence. The germline IGHV3-9 and IGKV2-28 sequences, and the amino acid position assignment numbers presented herein comport with the international Immunogenetics (IMGT) information system, as described in Lefranc, M.-P., et al., IMGT®, the international ImMunoGeneTics Information System®, Nucl. Acids Res, 37, D1006-D1012 (2009). In some embodiments, the exposed surface comprises a complementarity determining region (CDR). In some embodiments, the amino acid substitution or substitutions are selected from the group consisting of (a) a basic amino acid substituted for a neutral amino acid within CDR2 (e.g., at position 58) of IGHV3-9, (b) a neutral amino acid substituted for an acidic amino acid within CDR3 (e.g., at position 107) of IGHV3-9, and (c) a neutral amino acid substituted for a basic amino acid within CDR1 (e.g., at position 33) of IGKV2-28. Unique permutations in the charge distribution of an antibody, especially at an environmental interface (such as, e.g., in a CDR) would be expected to create unpredictable conditions for antibody stability in solution.

In some embodiments, the anti-hIL-4Rα antibody comprises at least one amino acid substitution, which creates a change in the torsional strain within a framework region of a variable region of the antibody relative to the germline IGHV3-9 sequence or the germline IGKV2-28 sequence. In some embodiments, the amino acid substitution or substitutions are selected from the group consisting of (a) a proline substituted for a non-proline amino acid in framework region 3 (FR3) (e.g., at position 96) of IGHV3-9, and (b) a non-proline amino acid substituted for a proline in framework region 2 (FR2) (e.g., at position 46) of IGKV2-28. Changes in the ability of the peptide chain to rotate, especially within a framework region, which affects the CDR interface with the solvent, would be expected to create unpredictable conditions for antibody stability in solution.

According to certain embodiments of the present disclosure, the anti-hIL-4Rα antibody, or antigen-binding fragment thereof, comprises a heavy chain complementary determining region (HCDR) 1 of SEQ ID NO: 2, an HCDR2 of SEQ ID NO:3, and an HCDR3 of SEQ ID NO: 4. In certain embodiments, the anti-hIL-4Rα antibody, or antigen-binding fragment thereof, comprises an HCVD of SEQ ID NO: 1.

According to certain embodiments of the present disclosure, the anti-hIL-4Rα, or antigen-binding fragment thereof, comprises a light (kappa) chain complementary determining region (LCDR) 1 of SEQ ID NO: 6, an LCDR2 of SEQ ID NO: 7, and an LCDR3 of SEQ ID NO: 8. In certain embodiments, the anti-hIL- 4Rα antibody, or antigen-binding fragment thereof, comprises an LCVD of SEQ ID NO: 5.

According to certain other embodiments of the present disclosure, the anti-hIL-4Rα antibody, or antigen-binding fragment thereof, comprises an HCDR1 of SEQ ID NO: 10, an HCDR2 of SEQ ID NO: 11, an HCDR 3 of SEQ ID NO: 12, an LCDR1 of SEQ ID NO: 14, an LCDR2 of SEQ ID NO: 15, and an LCDR3 of SEQ ID NO: 16. In certain embodiments, the anti-hIL- 4Rα antibody, or antigen-binding fragment thereof, comprises an HCVD of SEQ ID NO: 9 and an LCVD of SEQ ID NO: 13.

According to certain other embodiments of the present disclosure, the anti-hIL-4Rα antibody, or antigen-binding fragment thereof, comprises an HCDR1 of SEQ ID NO: 18, an HCDR2 of SEQ ID NO: 19, an HCDR3 of SEQ ID NO: 20, an LCDR1 of SEQ ID NO: 22, an LCDR2 of SEQ ID NO: 23, and an LCDR3 of SEQ ID NO: 24. In certain embodiments, the anti-hIL- 4Rα antibody, or antigen-binding fragment thereof, comprises an HCVD of SEQ ID NO: 17 and an LCVD of SEQ ID NO: 21.

Another non-limiting, exemplary antibody which may be used in the practice of this disclosure is referred to as “mAb2”. This antibody is also referred to in U.S. Pat. No. 7,608,693 as H4H083P. mAb2 (H4H083P) comprises an HCVR/LCVR amino acid sequence pair having SEQ ID NOs:9/13, and HCDR1-HCDR2-HCDR3/LCDR1-LCDR2-LCDR3 domains represented by SEQ ID NOs:10-11-12/SEQ ID NOs:14-15-16.

Yet another non-limiting, exemplary antibody which may be used in the practice of this disclosure is referred to as “mAb3”. This antibody is also referred to in U.S. Pat. No. 7,608,693 as H4H095P. mAb3 (H4H095P) comprises an HCVR/LCVR amino acid sequence pair having SEQ ID NOs:17/21, and HCDR 1-HCDR2-HCDR3/LCDR1-LCDR2-LCDR3 domains represented by SEQ ID NOs:18-19-20/SEQ ID NOs:22-23-24.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations are liquid formulations that may contain about 100±10 mg/mL to about 200±20 mg/mL of antibody; about 110±11 mg/mL to about 190±19 mg/mL of antibody; about 120±12 mg/mL to about 180±18 mg/mL of antibody; about 130±13 mg/mL to about 170±17 mg/mL of antibody; about 140±14 mg/mL to about 160±16 mg/mL of antibody; or about 150±15 mg/mL of antibody. For example, the formulations of the present disclosure may comprise about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 131 mg/mL; about 132 mg/mL; about 133 mg/mL; about 134 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to hIL-4Rα.

The pharmaceutical formulations of the present disclosure comprise one or more excipients. The term “excipient”, as used herein, means any non-therapeutic agent added to the formulation to provide a desired consistency, viscosity or stabilizing effect.

In certain embodiments, the pharmaceutical formulation of the disclosure comprises at least one organic cosolvent in a type and in an amount that stabilizes the hIL-4Rα antibody under conditions of rough handling, such as, e.g., vortexing. In some embodiments, what is meant by “stabilizes” is the prevention of the formation of more than 2% aggregated antibody of the total amount of antibody (on a molar basis) over the course of rough handling. In some embodiments, rough handling is vortexing a solution containing the antibody and the organic cosolvent for about 120 minutes.

In certain embodiments, the organic cosolvent is a non-ionic surfactant, such as an alkyl poly(ethylene oxide). Specific non-ionic surfactants that can be included in the formulations of the present disclosure include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 181, poloxamer 188, poloxamer 407; or polyethylene glycol (PEG). Polysorbate 20 is also known as TWEEN 20, sorbitan monolaurate and polyoxyethylenesorbitan monolaurate. Poloxamer 181 is also known as PLURONIC F68.

The amount of organic cosolvent contained within the pharmaceutical formulations of the present disclosure may vary depending on the specific properties desired of the formulations, as well as the particular circumstances and purposes for which the formulations are intended to be used. In certain embodiments, the formulations may contain about 0.1%±0.01% to about 2%±0.2% surfactant. For example, the formulations of the present disclosure may comprise about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% polysorbate 20 or poloxamer 181. For example, the formulations of the present disclosure may comprise about 0.5%; about 0.6%; about 0.7%; about 0.8%; about 0.9%; about 1%; about 1.1%; about 1.2%; about 1.3%; about 1.4%; about 1.5%; about 1.6%; about 1.7%; about 1.8%; about 1.9%; or about 2.0% PEG 3350.

Exemplary organic cosolvents that stabilize the hIL-4Rα antibody include 0.2%±0.02% polysorbate 20, 0.2%±0.02% poloxamer 181, or 1%±0.1% PEG 3350.

The pharmaceutical formulations of the present disclosure may also comprise one or more thermal stabilizers in a type and in an amount that stabilizes the hIL-4Rα antibody under conditions of thermal stress. In some embodiments, what is meant by “stabilizes” is maintaining greater than about 92% of the antibody in a native conformation when the solution containing the antibody and the thermal stabilizer is kept at about 45° C. for up to about 28 days. In some embodiments, what is meant by “stabilizes” is wherein less than about 5% of the antibody is aggregated when the solution containing the antibody and the thermal stabilizer is kept at about 45° C. for up to about 28 days.

In certain embodiments, the thermal stabilizer is a sugar or sugar alcohol selected from sucrose, trehalose and mannitol, or any combination thereof, the amount of which contained within the formulation can vary depending on the specific circumstances and intended purposes for which the formulation is used. In certain embodiments, the formulations may contain about 2.5% to about 10% sugar or sugar alcohol; about 3% to about 9.5% sugar or sugar alcohol; about 3.5% to about 9% sugar or sugar alcohol; about 4% to about 8.5% sugar or sugar alcohol; about 4.5% to about 8% sugar or sugar alcohol; about 5% to about 7.5% sugar or sugar alcohol; about 5.5% to about 7% sugar or sugar alcohol; or about 6.0% to about 6.5% sugar or sugar alcohol. For example, the pharmaceutical formulations of the present disclosure may comprise about 2.5%±0.375%; about 3%±0.45%; about 3.5%±0.525%; about 4.0%±0.6%; about 4.5%±0.675%; about 5.0%±0.75%; about 5.5%±0.825%; about 6.0%±0.9%; about 6.5%±0.975%; about 7.0%±1.05%; about 7.5%±1.125%; about 8.0%±1.2%; 8.5%±1.275%; about 9.0%±1.35%; or about 10.0%±1.5% sugar or sugar alcohol (e.g., sucrose, trehalose or mannitol).

The pharmaceutical formulations of the present disclosure may also comprise a buffer or buffer system, which serves to maintain a stable pH and to help stabilize the hIL-4Rα antibody. In some embodiments, what is meant by “stabilizes” is wherein less than 3.0%±0.5% of the antibody is aggregated when the solution containing the antibody and the buffer is kept at about 45° C. for up to about 14 days. In some embodiments, what is meant by “stabilizes” is wherein less than 3.7%±0.5% of the antibody is aggregated when the solution containing the antibody and the buffer is kept at about 25° C. for up to about 6 months. In some embodiments, what is meant by “stabilizes” is wherein at least 95%±0.5% of the antibody is in its native conformation as determined by size exclusion chromatography when the solution containing the antibody and the buffer is kept at about 45° C. for up to about 14 days. In some embodiments, what is meant by “stabilizes” is wherein at least 96%±0.5% of the antibody is in its native conformation as determined by size exclusion chromatography when the solution containing the antibody and the buffer is kept at about 25° C. for up to about 6 months. In some embodiments, what is meant by “stabilizes” is wherein at least 62%±0.5% of the antibody is in its neutral conformation as determined by cation exchange chromatography when the solution containing the antibody and the buffer is kept at about 45° C. for up to about 14 days. In some embodiments, what is meant by “stabilizes” is wherein at least 54%±0.5% of the antibody is in its neutral conformation as determined by cation exchange chromatography when the solution containing the antibody and the buffer is kept at about 25° C. for up to about 6 months. By “neutral conformation”, what is meant is the faction of antibody that elutes from an ion exchange resin in the main peak, which is generally flanked by more “basic” peaks on one side and more “acidic” peaks on the other side.

The pharmaceutical formulations of the present disclosure may have a pH of from about 5.2 to about 6.4. For example, the formulations of the present disclosure may have a pH of about 5.2; about 5.3; about 5.4; about 5.5; about 5.6; about 5.7; about 5.8; about 5.9; about 6.0; about 6.1; about 6.2; about 6.3; or about 6.4. In some embodiments, the pH is about 5.3±0.2; about 5.9±0.2; or about 6.0±0.2.

In some embodiments, the buffer or buffer system comprises at least one buffer that has a buffering range that overlaps fully or in part the range of pH 5.2-6.4. In one embodiment, the buffer or buffer system comprises two buffers, the first of which has an effective pH range within 3.6-5.6 and the second of which has an effective pH range within 5.5-7.4. In one embodiment, the first buffer has a pKa of about 4.8±0.3 and the second buffer has a pKa of about 6.0±0.3. In certain embodiments, the buffer system comprises an acetate buffer and a histidine buffer. In certain embodiments, the histidine is present at about 1.3-1.9 parts per 1 part of acetate by mole. In certain embodiments, the histidine is present at about 1.6±0.25 parts to 1 part of acetate by mole. In certain embodiments, the acetate is present at a concentration of about 2.5 mM to about 22.5 mM; about 3.0 mM to about 22 mM; about 3.5 mM to about 21.5 mM; about 4.0 mM to about 21.0 mM; about 4.5 mM to about 20.5 mM; about 5.0 mM to about 20 mM; about 5.5 mM to about 19.5 mM; about 6.0 mM to about 19.0 mM; about 6.5 mM to about 18.5 mM; about 7.0 mM to about 18.0 mM; about 7.5 mM to about 17.5 mM; about 8.0 mM to about 17 mM; about 8.5 mM to about 16.5 mM; about 9.0 mM to about 16.0 mM; about 9.5 mM to about 15.5 mM; about 10.0 mM to about 15.0 mM; about 10.5 mM to about 14.5 mM; about 12.5 mM±1.875 mM; about 11.0 mM to about 14.0 mM; about 11.5 mM to about 13.5 mM; or about 12.0 mM to about 13.0 mM. In certain embodiments, the histidine is present at a concentration of about 10 mM to about 30 mM; about 11 mM to about 29 mM; about 12 mM to about 28 mM; about 13 mM to about 27 mM; about 14 mM to about 26 mM; about 15 mM to about 25 mM; about 16 mM to about 24 mM; about 17 mM to about 23 mM; about 18 mM to about 22 mM; or about 19 mM to about 21 mM. In certain embodiments, the buffer system comprises acetate at about 12.5 mM and histidine at about 20 mM, at a pH of about 5.9.

The pharmaceutical formulations of the present disclosure may also comprise one or more excipients, which serve to maintain a reduced viscosity or to lower the viscosity of formulations containing a high concentration of protein (e.g., generally >100 mg/ml of protein). In some embodiments, the formulation comprises arginine in an amount sufficient to maintain the viscosity of the liquid formulation at less than about 35 cPoise, less than about 30 cPoise, less than about 25 cPoise, less than about 20 cPoise, less than about 15 cPoise, less than about 14 cPoise, less than about 13 cPoise, less than about 12 cPoise, less than about 10 cPoise, or less than about 9 cPoise.

In certain embodiments, the pharmaceutical formulation of the present disclosure contains arginine, preferably as L-arginine hydrochloride, at a concentration of about 25 mM±3.75 mM, about 50 mM±7.5 mM, or about 100 mM±15 mM. In certain embodiments, the arginine is at about 20 mM to about 30 mM, about 21 mM to about 29 mM, about 21.25 mM to about 28.75 mM, about 22 mM to about 28 mM, about 23 mM to about 27 mM or about 24 mM to about 26 mM.

According to one aspect of the present disclosure, the pharmaceutical formulation is a low viscosity, generally physiologically isotonic liquid formulation, which comprises: (i) a human antibody that specifically binds to hIL-4Rα (e.g., mAb1, mAb2 or mAb3 [supra]), at a concentration of about 100 mg/ml or greater; (ii) a buffer system that provides sufficient buffering at about 5.9±0.6; (iii) a sugar which serves inter alia as a thermal stabilizer; (iv) an organic cosolvent, which protects the structural integrity if the antibody; and (v) an amino acid, which serves to keep the viscosity manageable for subcutaneous injection.

According to one embodiment, the pharmaceutical formulation comprises: (i) a human IgG1 antibody that specifically binds to hIL-4Rα and which comprises a substituted IGHV3-9 type heavy chain variable region and a substituted IGLV2-28 type light chain variable region (e.g., mAb1) at a concentration from about 100 mg/ml to about 200 mg/ml; (ii) a buffer system comprising acetate and histidine, which buffers effectively at about pH 5.9±0.6; (iii) sucrose as a thermal stabilizer; (iv) a polysorbate as an organic cosolvent; and (v) arginine as a viscosity reducer.

According to one embodiment, the pharmaceutical formulation comprises: (i) a human IgG1 antibody that specifically binds to hIL-4Rα, and which comprises an HCDR1 of SEQ ID NO:2, an HCDR2 of SEQ ID NO:3, an HCDR3 of SEQ ID NO:4, an LCDR1 of SEQ ID NO:6, an LCDR2 of SEQ ID NO:7, and an LCDR 3 of SEQ ID NO:8, at a concentration of about 150 mg/ml±25 mg/ml; (ii) acetate at about 12.5 mM±1.9 mM and histidine at about 20 mM±3 mM, which buffers effectively at about pH 5.9±0.3; (iii) sucrose at about 5% w/v±0.75% w/v; (iv) polysorbate 20 at about 0.2% w/v±0.03% w/v; and (v) arginine as L-arginine hydrochloride at about 25 mM±3.75 mM.

The pharmaceutical formulations of the present disclosure typically exhibit high levels of stability. The term “stable”, as used herein in reference to the pharmaceutical formulations, means that the antibodies within the pharmaceutical formulations retain an acceptable degree of chemical structure or biological function after storage under defined conditions. A formulation may be stable even though the antibody contained therein does not maintain 100% of its chemical structure or biological function after storage for a defined amount of time. Under certain circumstances, maintenance of about 90%, about 95%, about 96%, about 97%, about 98% or about 99% of an antibody's structure or function after storage for a defined amount of time may be regarded as “stable”.

Stability can be measured, inter alia, by determining the percentage of native antibody that remains in the formulation after storage for a defined amount of time at a defined temperature. The percentage of native antibody can be determined by, inter alia, size exclusion chromatography (e.g., size exclusion high performance liquid chromatography [SE-HPLC]). An “acceptable degree of stability”, as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a defined temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The defined temperature at which the pharmaceutical formulation may be stored when assessing stability can be any temperature from about −80° C. to about 45° C., e.g., storage at about −30° C., about −20° C., about 0 ° C., about 4°-8° C., about 5° C., about 25° C., or about 45° C. For example, a pharmaceutical formulation may be deemed stable if after 3 months of storage at 5° C., greater than about 90%, 95%, 96%, 97% or 98% of native antibody is detected by SE-HPLC. A pharmaceutical formulation may also be deemed stable if after 6 months of storage at 5° C., greater than about 90%, 95%, 96%, 97% or 98% of native antibody is detected by SE-HPLC. A pharmaceutical formulation may also be deemed stable if after 9 months of storage at 5° C., greater than about 90%, 95%, 96%, 97% or 98% of native antibody is detected by SE-HPLC. A pharmaceutical formulation may also be deemed stable if after 3 months of storage at 25° C., greater than about 90%, 95%, 96% or 97% of native antibody is detected by SE-HPLC. A pharmaceutical formulation may also be deemed stable if after 6 months of storage at 25° C., greater than about 90%, 95%, 96% or 97% of native antibody is detected by SE-HPLC. A pharmaceutical formulation may also be deemed stable if after 9 months of storage at 25° C., greater than about 90%, 95%, 96% or 97% of native antibody is detected by SE-HPLC.

Stability can be measured, inter alia, by determining the percentage of antibody that forms in an aggregate within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC]). An “acceptable degree of stability”, as that phrase is used herein, means that at most 5% of the antibody is in an aggregated form detected in the formulation after storage for a defined amount of time at a given temperature. In certain embodiments an acceptable degree of stability means that at most about 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −30° C., about −20° C., about 0 ° C., about 4°-8° C., about 5° C., about 25° C., or about 45° C. For example, a pharmaceutical formulation may be deemed stable if after 3 months of storage at 5° C., less than about 5%, 4%, 3%, 2%, 1%, 0.5%, or 0.1% of the antibody is detected in an aggregated form. A pharmaceutical formulation may also be deemed stable if after 6 months of storage at 5° C., less than about 5%, 4%, 3%, 2%, 1%, 0.5%, or 0.1% of the antibody is detected in an aggregated form. A pharmaceutical formulation may also be deemed stable if after 9 months of storage at 5° C., less than about 5%, 4%, 3%, 2%, 1%, 0.5%, or 0.1% of the antibody is detected in an aggregated form. A pharmaceutical formulation may also be deemed stable if after 3 months of storage at 25° C., less than about 5%, 4%, 3%, 2%, 1%, 0.5%, or 0.1% of the antibody is detected in an aggregated form. A pharmaceutical formulation may also be deemed stable if after 6 months of storage at 25° C., less than about 5%, 4%, 3%, 2%, 1%, 0.5%, or 0.1% of the antibody is detected in an aggregated form. A pharmaceutical formulation may also be deemed stable if after 9 months of storage at 25° C., less than about 5%, 4%, 3%, 2%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“neutral conformation”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” or “deamidated” antibody can be determined by, inter alia, ion exchange chromatography (e.g., cation exchange high performance liquid chromatography [CEX-HPLC]). An “acceptable degree of stability”, as that phrase is used herein, means that at most 45% of the antibody is in a more acidic form detected in the formulation after storage for a defined amount of time at a defined temperature. In certain embodiments an acceptable degree of stability means that at most about 45%, 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The temperature at which the pharmaceutical formulation may be stored when assessing stability can be any temperature from about −80° C. to about 45° C., e.g., storage at about −30° C., about −20° C., about 0 ° C., about 4°-8° C., about 5° C., about 25° C., or about 45° C. For example, a pharmaceutical formulation may be deemed stable if after 3 months of storage at 5° C., less than about 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 3 months of storage at 25° C., less than about 18%, 17%, 16%, 15%, 14%, 13%, 12%, 10%, 9%, 8%, 7%, 6%, 5%, 4%, 3%, 2%, 1%, 0.5% or 0.1 of the antibody is in a more acidic form. A pharmaceutical formulation may also be deemed stable if after 8 weeks of storage at 45° C., less than about 45%, 40%, 35%, 30%, 25%, 20%, 15%, 10%, 5%, 4%, 3%, 2%, 1%, 0.5%, or 0.1% of the antibody is in a more acidic form. A pharmaceutical formulation may also be deemed stable if after 2 weeks of storage at 40° C., less than about 20%, 19%, 18%, 17%, 16%, 15%, 14%, 13%, 12%, 11%, 10%, 9%, 8%, 7%, 6%, 5%, 4%, 3%, 2%, 1%, 0.5%, or 0.1% of the antibody can be detected in a more acidic form.

Other methods may be used to assess the stability of the formulations of the present disclosure such as, e.g., differential scanning calorimetry (DSC) to determine thermal stability, controlled agitation to determine mechanical stability, and absorbance at about 350 nm or about 405 nm to determine solution turbidities. For example, a formulation of the present disclosure may be considered stable if, after 6 or more months of storage at about 5° C. to about 25° C., the change in OD405 of the formulation is less than about 0.05 (e.g., 0.04, 0.03, 0.02, 0.01, or less) from the OD405 of the formulation at time zero.

Stability may also be assessed by measuring the biological activity or binding affinity of the antibody to its target. For example, a formulation of the present disclosure may be regarded as stable if, after storage at e.g., 5° C., 25° C., 45° C., etc. for a defined amount of time (e.g., 1 to 12 months), the anti-IL-4Rα antibody contained within the formulation binds to IL-4Rα with an affinity that is at least 90%, 95%, or more of the binding affinity of the antibody prior to said storage. Binding affinity may be determined by e.g., ELISA or plasmon resonance. Biological activity may be determined by an IL-4Rα activity assay, such as e.g., contacting a cell that expresses IL-4Rα with the formulation comprising the anti IL-4Rα antibody. The binding of the antibody to such a cell may be measured directly, such as e.g., via FACS analysis. Alternatively, the downstream activity of the IL-4Rα system may be measured in the presence of the antibody and an IL-4Rα agonist, and compared to the activity of the IL-4Rα system in the absence of antibody. In some embodiments, the IL-4Rα may be endogenous to the cell. In other embodiments, the IL-4Rα may be ectopically expressed in the cell.

The liquid pharmaceutical formulations of the present disclosure may, in certain embodiments, exhibit low to moderate levels of viscosity. “Viscosity” as used herein may be “kinematic viscosity” or “absolute viscosity”. “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. For example, if one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density (Absolute Viscosity=Kinematic Viscosity×Density). The dimension of kinematic viscosity is L2/T where L is a length and T is a time. Commonly, kinematic viscosity is expressed in centistokes (cSt). The SI unit of kinematic viscosity is mm 2/s, which is 1 cSt. Absolute viscosity is expressed in units of centipoise (cP). The SI unit of absolute viscosity is the milliPascal-second (mPa-s), where 1 cP=1 mPa-s.

As used herein, a low level of viscosity, in reference to a fluid formulation of the present disclosure, will exhibit an absolute viscosity of less than about 15 cPoise (cP). For example, a fluid formulation of the disclosure will be deemed to have “low viscosity”, if, when measured using standard viscosity measurement techniques, the formulation exhibits an absolute viscosity of about 15 cP, about 14 cP, about 13 cP, about 12 cP, about 11 cP, about 10 cP, about 9 cP, about 8 cP, or less. As used herein, a moderate level of viscosity, in reference to a fluid formulation of the present disclosure, will exhibit an absolute viscosity of between about 35 cP and about 15 cP. For example, a fluid formulation of the disclosure will be deemed to have “moderate viscosity”, if when measured using standard viscosity measurement techniques, the formulation exhibits an absolute viscosity of about 34 cP, about 33 cP, about 32 cP, about 31 cP, about 30 cP, about 29 cP, about 28 cP, about 27 cP, about 26 cP, about 25 cP, about 24 cP, about 23 cP, about 22 cP, about 21 cP, about 20 cP, about 19 cP, 18 cP, about 17 cP, about 16 cP, or about 15.1 cP.

The pharmaceutical formulations of the present disclosure are useful, inter alia, for the treatment, prevention or amelioration of any disease or disorder associated with IL-4 activity, including diseases or disorders mediated by activation of IL-4Rα. Exemplary, non-limiting diseases and disorders that can be treated or prevented by the administration of the pharmaceutical formulations of the present disclosure include various atopic diseases such as, e.g., atopic dermatitis, allergic conjunctivitis, allergic rhinitis, asthma and other IgE/Th2 mediated diseases.

Thus, the present disclosure includes methods of treating, preventing, or ameliorating any disease or disorder associated with IL-4 activity or IL-4Rα activation (including any of the above mentioned exemplary diseases, disorders and conditions). The therapeutic methods of the present disclosure comprise administering to a subject any formulation comprising an anti-hIL-4Rα antibody as disclosed herein. The subject to which the pharmaceutical formulation is administered can be, e.g., any human or non-human animal that is in need of such treatment, prevention or amelioration, or who would otherwise benefit from the inhibition or attenuation of IL-4 or IL-4Rα-mediated activity. For example, the subject can be an individual that is diagnosed with, or who is deemed to be at risk of being afflicted by any of the aforementioned diseases or disorders. The present disclosure further includes the use of any of the pharmaceutical formulations disclosed herein in the manufacture of a medicament for the treatment, prevention or amelioration of any disease or disorder associated with IL-4 activity or IL-4Rα activation (including any of the above mentioned exemplary diseases, disorders and conditions).

According to one aspect of the present disclosure, the pharmaceutical formulation is a stable, low viscosity, generally physiologically isotonic liquid formulation, which comprises: (i) a human antibody that specifically binds to human PD-1 (e.g., H4H7798N), at a concentration of up to 250 mg/mL±45 mg/mL; (ii) a histidine buffer system that provides sufficient buffering at about pH 6.0±0.3; (iii) an organic cosolvent, which protects the structural integrity of the antibody; (iv) a thermal stabilizer that is a sugar; and (iv) a viscosity modifier that is an amino acid, which serves to keep the viscosity manageable for injection in a convenient volume for subcutaneous administration.

According to one embodiment, the stable, low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of up to 200 mg/ml±30 mg/mL; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 175 mg/ml±26.25 mg/mL; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR 3 of SEQ ID NO: 8, at a concentration of 150 mg/ml±22.5 mg/mL; (ii) histidine buffer at 10 mM±2 mM, which buffers at pH 6.0±0.3; (iii) polysorbate 80 at 0.2% w/v±0.1% w/v; (iv) sucrose at 5%±1% w/v; and (v) L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR 1 of SEQ ID NO: 6, an LCDR2 of SEQ ID NO: 7, and an LCDR3 of SEQ ID NO: 8, at a concentration of 100 mg/mL±15 mg/mL; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 50 mg/mL±7.5 mg/mL; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

According to one embodiment, the stable low-viscosity pharmaceutical formulation comprises: (i) a human IgG4 antibody that specifically binds to human PD-1, and which comprises an HCDR1 of SEQ ID NO: 3, an HCDR2 of SEQ ID NO: 4, an HCDR3 of SEQ ID NO: 5, an LCDR1 of SEQ ID NO: 6, an LCDR2 of SEQ ID NO: 7, and an LCDR3 of SEQ ID NO: 8, at a concentration of 25 mg/mL±3.75 mg/mL; (ii) histidine buffer at 10 mM±2 mM, which buffers at pH 6.0±0.3; (iii) sucrose at 5% w/v±1% w/v; (iv) polysorbate 80 at 0.2% w/v±0.1%; and L-proline at 1.5% (w/v)±0.3%.

Additional exemplary medicaments, drugs, and/or pharmaceutical formulations include: pharmaceuticals targeting Activin A and GDF8 (e.g., garetosmab and trevogrumab as described in U.S. Pat. No. 9,718,881, which is incorporated by reference herein); pharmaceuticals targeting C5 (e.g., pozelimab and cemdisiran, as described in US Publication 2021/0046182, which is incorporated by reference herein); pharmaceuticals targeting LEPR (e.g., mibavademab, as described in U.S. Pat. No. 10,023,644, which is incorporated by reference herein); pharmaceuticals targeting LAG3 (e.g., fianlimab, as described in U.S. Pat. No. 10,358,495, which is incorporated by reference herein); pharmaceuticals targeting BetV1 (e.g., antibodies disclosed in U.S. Pat. No. 10,793,624, which is incorporated by reference herein); pharmaceuticals targeting PCSK9 (e.g., alirocumab, as described in U.S. Pat. No. 8,795,669, which is incorporated by reference herein); pharmaceuticals targeting ANGPTL3 (e.g., evinacumab, as disclosed in US Publication 2020/0369760, which is incorporated by reference herein); pharmaceuticals targeting Ebola (e.g., atolivimab, maftivimab, odesivimab, as disclosed in US Publication 2021/0252146, which is incorporated by reference herein); pharmaceuticals targeting IL-6R (e.g., sarilumab, as disclosed in U.S. Pat. No. 9,173,880, which is incorporated by reference herein).

Additional exemplary medicaments, drugs, and/or pharmaceutical formulations include: RNAi therapeutic targeting APP for early-onset Alzheimer's disease (ALN-APP1); RNAi therapeutic targeting HSD17B13 for nonalcoholic steatohepatitis (“NASH”) (ALN-HSD); RNAi therapeutic targeting PNPLA3 for NASH (ALN-PNP1); PD-1 Antibody for First-line NSCLC, BNT116 combination (CEMIPLIMAB); Bispecific antibody targeting BCMA and CD3 for Multiple myeloma (LINVOSELTAMAB); TTR gene knockout using CRISPR/Cas9 for Transthyretin (“ATTR”) amyloidosis; Bispecific antibody targeting CD20 and CD3 for certain B-cell malignancies (ODRONEXTAMAB); Bispecific antibody targeting PSMA and CD3 for prostate cancer; Bispecific antibody targeting two distinct MET epitopes for MET-altered advanced NSCLC; Bispecific antibody-drug conjugate targeting two distinct MET epitopes for MET overexpressing advanced cancer; Agonist Antibody to NPR1/Reversal Agent to REGN5381 for reversal agent in healthy volunteers; Bispecific antibody targeting BCMA and CD3 for transplant desensitization in patients with chronic kidney disease; Bispecific antibody targeting MUC16 and CD28 for platinum-resistant ovarian cancer; Bispecific antibody targeting PSMA and CD28 for prostate cancer; Bispecific antibody targeting CD22 and CD28 for B-NHL; Antibody to GITR for solid tumors; Bispecific antibody targeting EGFR and CD28 for solid tumors; Antibody to IL2Rg for aplastic anemia; Antibody to Factor XI for thrombosis; Antibody to TMPRSS6 for transfusion dependent iron overload; Antibody to Factor XI for thrombosis; RNAi therapeutic targeting HSD17B13 for nonalcoholic steatohepatitis (“NASH”) (ALN-HSD); Antibody to PD-1 Neoadjuvant CSCC; for second-line cervical cancer, ISA101b combination (CEMIPLIMAB); Antibody to IL-4R alpha subunit for ulcerative colitis; Eosinophilic gastroenteritis (Phase 2/3) (DUPILUMAB); Antibody to LAG-3 for first-line advanced NSCLC (Phase 2/3) (pivotal study) (FIANLIMAB); Bispecific antibody targeting BCMA and CD3 for multiple myeloma (pivotal study) (LINVOSELTAMAB); Agonist antibody to leptin receptor (“LEPR”) for generalized lipodystrophy; Partial lipodystrophy (MIBAVADEMAB); Bispecific antibody targeting CD20 and CD3 for B-cell non-Hodgkin lymphoma (“B-NHL”) (pivotal study) (ODRONEXTAMAB); Antibody to C5; studied as monotherapy and in combination with cemdisiran for CD55-deficient protein-losing enteropathy (“CHAPLE”), monotherapy (potentially pivotal study) (POZELIMAB); Agonist Antibody to NPR1/Reversal Agent to REGN5381 for heart failure; Antibody to IL-6R for polyarticular-course juvenile idiopathic arthritis (“pcJIA”) (pivotal study); systemic juvenile idiopathic arthritis (“sJIA”) (pivotal study) (SARILUMAB); Bispecific antibody targeting MUC16 and CD3 for platinum-resistant ovarian cancer (UBAMATAMAB); Immune activator targeting TLR9 for solid tumors (VIDUTOLIMOD); VEGF-Trap for Wet AMD, DME (AFLIBERCEPT); Antibody to PCSK9 for HeFH in pediatrics (ALIROCUMAB); Antibody to PD-1 for adjuvant CSCC (CEMIPLIMAB); Antibody to IL-4R alpha subunit for EoE in pediatrics; chronic obstructive pulmonary disease (“COPD”); bullous pemphigoid; chronic spontaneous urticaria (“CSU”); chronic pruritis of unknown origin (DUPILUMAB); Antibody to LAG-3 for first-line metastatic melanoma; First-line adjuvant melanoma (FIANLIMAB); Antibody to Activin A for fibrodysplasia ossificans progressiva (“FOP”) (GARETOSMAB); Antibody to IL-33 for COPD (ITEPEKIMAB); Antibody to C5; studied as monotherapy and in combination with cemdisiran for myasthenia gravis, cemdisiran combination; paroxysmal nocturnal hemoglobinuria (“PNH”), cemdisiran combination (POZELIMAB); Multi-antibody therapy to Bet v 1 for birch allergy; Antibody that binds to and inhibits both vascular endothelial growth factor (VEGF)-F and angiopoietin-2 (Ang-2) (faricimab-svoa; VABYSMO).

The product containers, e.g., syringes, described herein may have any suitable volume depending on the drug to be delivered. For example, the volume of the product containers, e.g., syringes, may range from about 1 mL to about 50 mL, from about 2 mL to about 10 mL, from about 3 mL to about 6 mL, or from about 2 mL to about 5 mL. In other examples, the product containers, e.g., syringes, may have a volume greater than or equal to about 1 mL, greater than or equal to about 1.25 mL, greater than or equal to about 1.5 mL, greater than or equal to about 1.75 mL, greater than or equal to about 2 mL, greater than or equal to about 2.25 mL, greater than or equal to about 2.5 mL, greater than or equal to about 2.75 mL, greater than or equal to about 3 mL, greater than or equal to about 3.25 mL, greater than or equal to about 3.5 mL, greater than or equal to about 3.75 mL, or greater than or equal to about 4 mL. In some examples, the product containers, e.g., syringes, may have a volume of greater than or equal to about 10 mL or greater than or equal to about 15 mL.

The devices of the present disclosure may also be configured to deliver additional components to and/or or around an injection site of the patient. For example, the additional components may include at least one of a film (e.g., a liquid bandage or surgical glue), an antiseptic, or an anesthetic. In some examples, a liquid bandage may be applied to and/or around an injection site of the patient. A liquid bandage may include a polymer dissolved in a solvent carrier, e.g., water or alcohol. The liquid bandage may protect the skin of the injection site and/or the skin around the injection site by forming a thin film of polymer on and/or around the injection site when the solvent carrier evaporates. In some examples, a film, e.g., a liquid bandage or surgical glue, may be applied to and/or around an injection site of the patient in combination with at least one of an antiseptic and anesthetic. Such components described above may be delivered to and/or around an injection site of the patient prior to and/or after delivery of the medicament. In other examples, such components may be delivered to and/or around an injection site of the patient after delivery of the medicament and prior to the lock-out configuration of the device. In some embodiments, the devices of the present disclosure may deliver a film to and/or around an injection site of the patient after delivery of the medicament and prior to the lock-out configuration of the device. Such additional components may be stored in a product container, e.g., syringe, of the devices disclosed herein. In other examples, any additional components may be stored in cap 232. In some examples, the devices disclosed herein may include at least one additional compartment, e.g., a chamber, to hold any additional components.

### Drug Delivery Training Devices

Embodiments of the present disclosure further relate to training devices, and in particular, training devices that simulate the experience of using a drug delivery device (e.g., a pre-filled syringe) safety system.

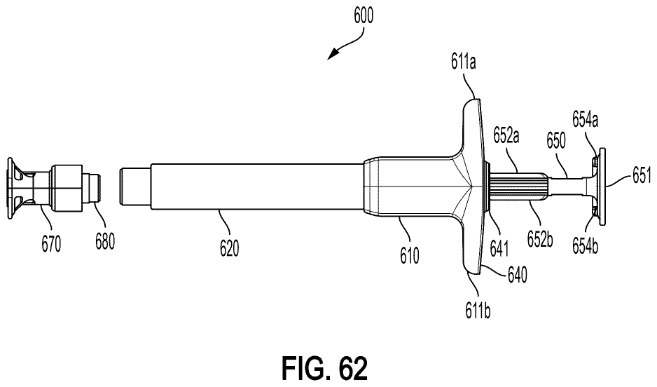

Referring now to FIGS. 62-70C, provided herein is an exemplary training device 600 and components of the same. The training device 600 includes a first housing 610, a second housing 620, a primary container 630, a rear cap 640, a plunger rod 650, and a piston 660. The first housing 610 may include a plurality of wings 611a, 611b. The second housing 620 may include a plurality of wings 621a, 621b. Each wing 621a, 621b may have a chamfered surface 622a, 622b, respectively. The primary container 630 may include an imitation needle 631, a tip 632, and a spring 633. The rear cap 640 may include a collar 641, a plurality of arms 642a, 642b, and a plurality of tabs 643a, 643b. The collar 641 may include a plurality of slots 644a, 644b. Each arm 642a, 642b may have a filleted inner surface 645a, 645b and a chamfered outer surface 646a, 646b, respectively. Each tab 643a, 643b may have a filleted inner surface 647a, 647b, respectively. The plunger rod 650 may include a pad 651, a plurality of step protrusions 652a, 652b, a cylindrical protrusion 653, a proximal portion 654, and a distal portion 655. The plurality of step protrusions 652a, 652b may have a plurality of chamfered distal surfaces 656a, 656b and a plurality of chamfered proximal surfaces 657a, 657b. The pad 651 may have a plurality of extrusions 658a, 658b. The piston 660 may be a single component. The training device 600 may optionally include a cap 670 and/or a needle shield 680. The cap 670 may be a single component. The needle shield 680 may be at least partially housed within the cap 670.

Components of the devices described herein may be made of any suitable material, and each component may be made from the same or different materials as other components. For example, one or more components may be made of a material including a polymer, such as a plastic. In some embodiments, one or more components may include multiple different materials, e.g., glass, poly(methyl methacrylate), any other suitable polymer or copolymer, plastic, or rubber. In some embodiments, a portion of the product container configured to contain a formulated drug substance may be made of a transparent or translucent material. In some embodiments, components of the device may be made of materials each having a similar or different hardness. In some embodiments, components may include elastic materials. For example, components that may be deflectable or flexible, may be made of a material having some flexibility, e.g., to allow for deflection. One or more of the materials listed above (e.g., plastic, rubber, polymers, or copolymers) may have such characteristics.

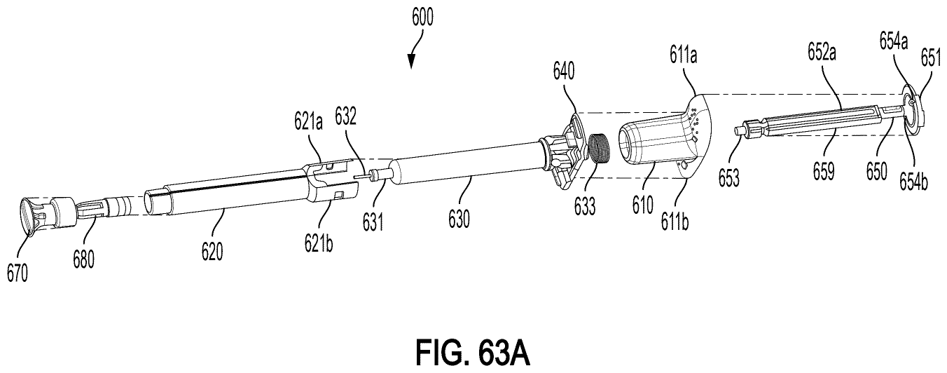

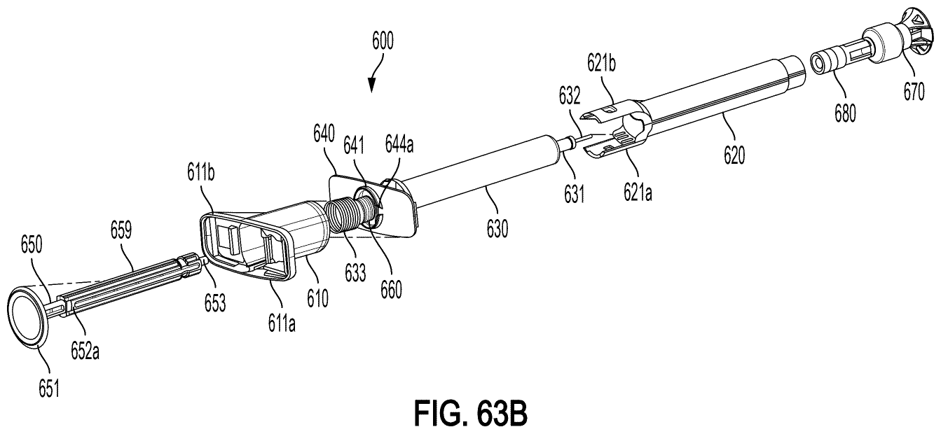

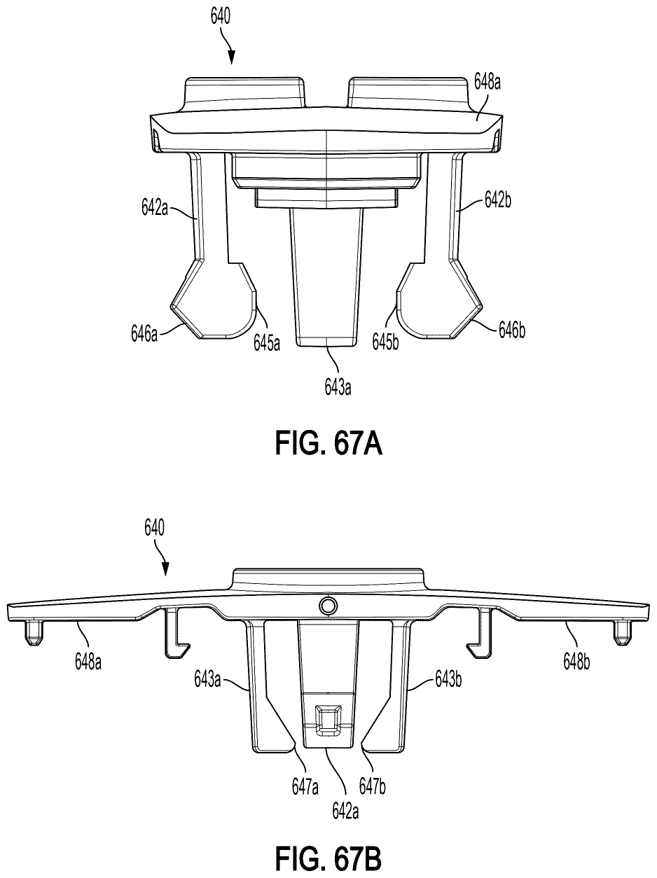

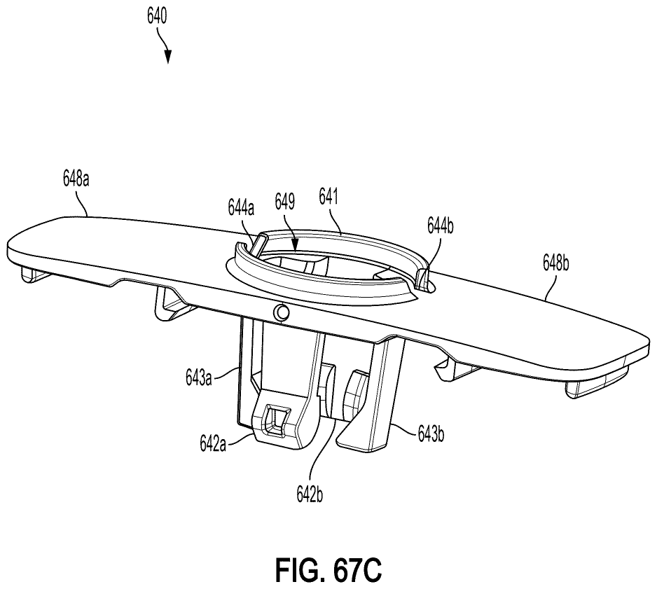

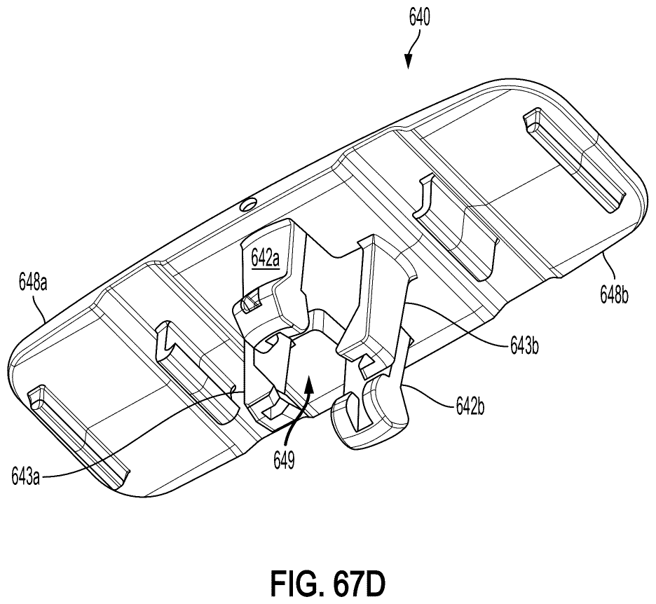

Referring now to FIGS. 62, 63A, and 63B, the training device 600 may comprise a first housing 610, a second housing 620 partially housed within the first housing 610, a primary container 630, a rear cap 640 partially housed within the first housing 610 and the second housing 620, and a plunger rod 650 partially housed within the first housing 610 and the second housing 620. The second housing 620 may comprise a proximal end and a distal end opposite the proximal end, wherein the proximal end comprises a plurality of wings 621a, 621b comprising chamfered proximal surfaces 622a, 622b. As shown in greater detail in FIGS. 67A-67D, the rear cap 640 may comprise a collar 641, a plurality of arms 642a, 642b, a plurality of tabs 643a, 643b, and a plurality of wings 648a, 648b. Each of the plurality arms 642a, 642b may comprise a chamfered outer surface 646a, 646b and each of the plurality of tabs may comprise a filleted inner surface 647a, 647b. The plunger rod 650 may comprise a body, a proximal end, and a distal end opposite to the proximal end. The proximal end may comprise a pad 651. The body may comprise a plurality of step protrusions 652a, 652b having a plurality of chamfered distal surfaces 656a, 656b and a plurality of chamfered proximal surfaces 657a, 657b. The device 600 may further comprise a piston 660, a cap 670, and a needle shield 680. The piston 660 may be attached to the plunger rod 650 and housed within the primary container 630. The cap 670 may be configured to at least partially receive the distal end of the second housing 620 and to at least partially receive the needle shield 680.

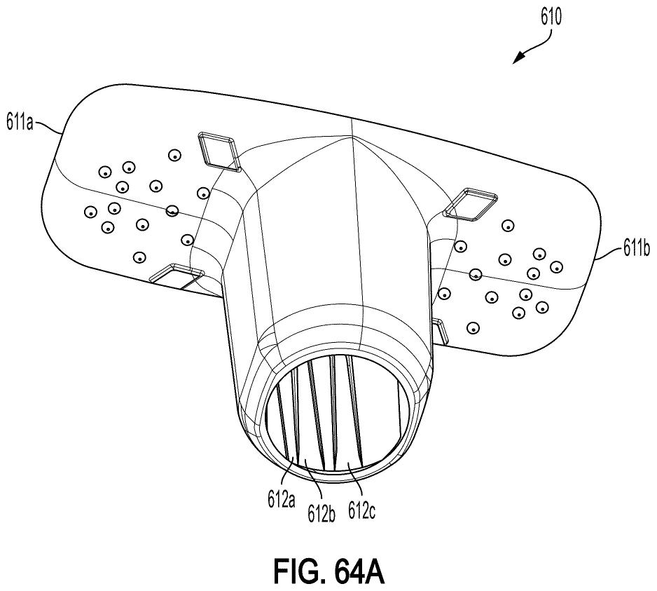

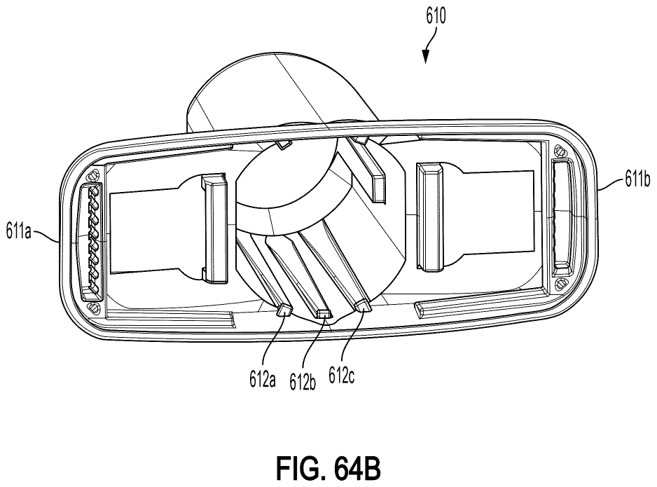

Referring now to FIGS. 64A-64B, the first housing 610 may comprise a proximal end, a distal end opposite to the proximal end, and a plurality of wings 611a, 611b. The first housing 610 may further comprise an internal opening spanning from the proximal end to the distal end. At its distal end, the first housing 610 may be configured to partially receive the second housing 620 and allow for proximal and/or distal movement of the second housing 620 within the first housing 610. The first housing 610 may comprise features that prevent unintended radial movement of wings 621a, 621b of the second housing 620. For example, the first housing 610 may comprise ramps 612a, 612b, 612c and ramps 612d, 612e, 612f (not shown) configured to contact the plurality of wings 621a, 621b of the second housing 620. At its proximal end, the first housing 610 may be configured to receive the rear cap 640, and the plurality of wings 611a, 611b may be configured to receive the plurality of wings 648a, 648b of the rear cap 640.

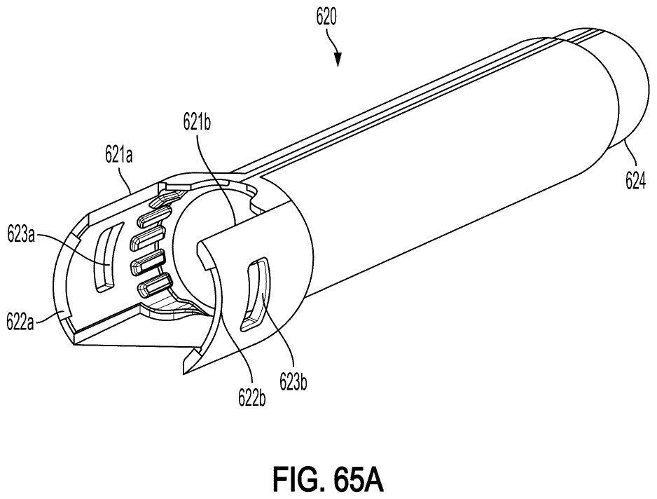

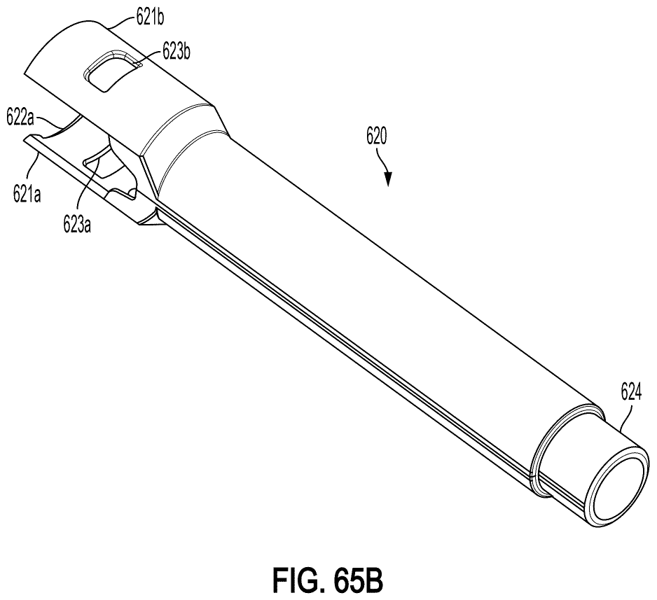

Referring now to FIGS. 65A-65B, the second housing 620 may be a monolithic or cylindrical shape, and may comprise a proximal end, a distal end opposite the proximal end, and an opening spanning from the proximal end to the distal end. At its proximal end, the second housing 620 may comprise a plurality of wings 621a, 621b, each having a chamfered surface 622a, 622b and a window 623a, 623b. When the second housing 620 is in a maximal distal position and moved proximally, the chamfered surfaces 622a, 622b may contact the plurality of arms 642a, 642b of the rear cap 640, applying a force to the plurality of arms 642a, 642b, with the chamfer of the chamfered surfaces 622a, 622b allowing for inward movement of the plurality of arms 642a, 642b. In this manner, the second housing 620 may bypass the plurality of arms 642a, 642b until the plurality of wings 621a, 621b contact the plurality of wings 648a, 648b of the rear cap 640. At this point, each window 623a, 623b may receive each arm of the plurality of arms 642a, 642b of the rear cap 640, allowing for the second housing 620 to be maintained at a maximal proximal position in the first housing 610. At its distal end, the second housing 620 may comprise a distal portion 624 having a smaller diameter than the rest of the second housing 620. The distal portion 624 may be configured to be received by a cap 670 and/or a needle shield 680. The second housing 620 may be transparent, so as to allow a user to see the piston 660 housed within the primary container 630, simulating a visual experience of a drug delivery device in which a user is able to view a piston while depressing a plunger rod.

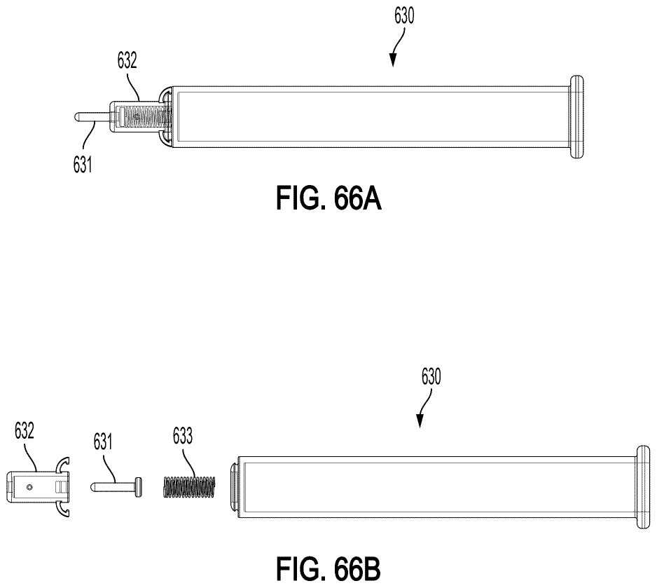

Referring now to FIGS. 66A-66B, the primary container 630 may have a monolithic and/or cylindrical shape, and may comprise a proximal end, a distal end opposite to the proximal end, and an opening spanning from the proximal end to the distal end. The primary container 630 may further comprise an imitation needle 631, a tip 632, and a spring 633. The tip 632 may be attached to the primary container 630, the spring 633 may be housed within the tip 632, and the imitation needle 631 may be attached to the spring 633 and housed within the tip 632. Contact between the imitation needle 631 and an external surface may cause compression of the spring 633 and retraction of the imitation needle 631 into the tip 632. The tip 632 is attached to the primary container 630 via a snap-on connection. In other embodiments, the tip 632 may be attached to the primary container 630 via other connections, such as a threaded connection, an adhesive connection, or the like.

Referring now to FIGS. 67A-67D, the rear cap 640 may comprise a proximal surface and a distal surface opposite to the proximal surface. The rear cap 640 may comprise an opening 649 spanning from the proximal surface to the distal surface, with a collar 641 surrounding the opening 649 on the proximal surface of the rear cap 640. The collar 641 may comprise a plurality of slots 644a, 644b configured to receive a plurality of extrusions 658a, 658b on the pad 651 of the plunger rod 650. The plurality of slots 644a, 644b may allow for a specific alignment of the plunger rod 650 to the rear cap 640 when the plunger rod 650 is moved into a maximal distal position. The rear cap 640 may further comprise the plurality of arms 642a, 642b and the plurality of tabs 643a, 643b, extending from the distal surface of the rear cap 640. Each of the plurality of arms 642a, 642b may comprise a filleted inner surface 645a, 645b and a chamfered outer surface 646a, 646b. Each of the plurality of tabs 643a, 643b may comprise a filleted inner surface 647a, 647b.

The plurality of arms 642a, 642b and the plurality tabs 643a, 643b may provide for simulation of safety features of a drug delivery device. For example, the plurality of filleted inner surfaces 645a, 645b of the plurality of arms 642a, 642b may be configured to contact the plunger rod 650, and the portion of the plunger rod 650 in contact with the plurality of arms 642a, 642b may change as the plunger rod 650 moves proximally from a maximal proximal position to a maximal distal position. When the plunger rod 650 is in a maximal proximal position, the plurality of arms 642a, 642b may contact step protrusions 652a, 652b of the plunger rod 650, described further below, preventing unintended radial movement of the plurality of arms 642a, 642b out of the windows 623a, 623b of the second housing 620 when the plunger rod 650 is not in a maximal distal position. In this manner, the plurality of arms 642a, 642b may prevent the second housing 620 from extending distally over the primary container 630 and/or the imitation needle 631.

In addition the plurality of filleted inner surfaces 647a, 647b of the plurality of tabs 643a, 643b may be configured to contact the plunger rod 650, and the portion of the plunger rod 650 in contact with the plurality of tabs 643a, 643b may change as the plunger rod 650 moves proximally from a maximal proximal position to a maximal distal position. When the plunger rod 650 is in a maximal proximal position, the plurality of tabs 643a, 643b may contact a distal portion 655 of the plunger rod 650 and/or a plurality of chamfered distal surfaces 656a, 656b of the plunger rod 650, described further below, preventing unintended distal movement of the plunger rod 650 when in a maximal proximal position. In this manner, the plurality of tabs 643a, 643b do not permit the plunger rod 650 to be fully removed from the device 600, simulating a safety feature of a drug delivery device which prevents loss of a plunger rod and maintains sterility of portion of the plunger rod in contact with a drug substance. To override this feature and allow for distal movement of the plunger rod 650, force may be applied sufficient to overcome frictional forces caused by contact between the plurality of tabs 643a, 643b and the plurality of chamfered distal surfaces 656a, 656b and/or the distal portion 655 of the plunger rod 650.

The plurality of arms 642a, 642b and the plurality tabs 643a, 643b may further provide for simulation of another safety feature of a drug delivery device, often referred to as a “lockout mechanism,” which prevents accidental access to a needle of a drug delivery device and prevents dangerous reuse of such a device. For example, as the plunger rod 650 approaches a maximal distal position, the filleted inner surfaces 645a, 645b of the plurality of arms 642a, 642b of the rear cap 640 may contact a proximal portion 654 of the plunger rod 650. Contact between the filleted inner surfaces 645a, 645b and the proximal portion 654 of the plunger rod 650 causes the plurality of arms 642a, 642b to flex inward, toward the plunger rod 650, such that they are no longer received by the windows 623a, 623b of the plurality of wings 621a, 621b of the second housing 620. The second housing 620, no longer anchored in place by the interaction of the plurality of arms 642a, 642b in the windows 623a, 623b, may then move distally to extend over the imitation needle 631. When the second housing 620 is in a maximal distal position, the imitation needle 631 is at least partially covered by the second housing 620, simulating an aspect of a lockout mechanism of a drug delivery device. In addition or alternatively, when the plunger rod 650 reaches a maximal distal position, the plurality of tabs 643a, 643b are no longer in contact with the step protrusions 652a, 652b, causing the plurality of tabs 643a, 643b to relax inward to contact a proximal portion 654 of the plunger rod 650. Contact between the plurality of tabs 643a, 643b and the proximal portion 654 of the plunger rod 650 may prevent proximal movement of the plunger rod 650. In this manner, the training device 600 is put in a lockout configuration that simulates a lockout configuration of a drug delivery device.

In addition, or alternatively, the plurality of tabs 643a, 643b may generate audible feedback (e.g., click, tapping, or other sound) to signify completion of simulated dose delivery. For example, contact between the plurality of tabs 643a, 643b and the proximal portion 654 of the plunger rod 650 may generate audible feedback such as a click sound. Such audible feedback may simulate a similar feature of a drug delivery device.

Unlike a drug delivery device, the training device 600 may include features that permit bypassing of a lockout mechanism, so as to permit continued practice and reuse of the device 600. One or more of such features may be referred to herein as a “reset mechanism”. For example, when the device 600 is in a lockout configuration wherein the plunger rod 650 is in a maximal distal position and the second housing 620 is in a maximal distal position, the chamfered outer surfaces 646a, 646b of the plurality of arms 642a, 642b may contact the chamfered surfaces 622a, 622b of the plurality of wings 621a, 621b of the second housing 620. A user may apply a force to the second housing 620 in a proximal direction, sufficient to overcome friction between the sloped shape of chamfered outer surfaces 646a, 646b of the plurality of arms 642a, 642b and the sloped shape of the chamfered surfaces 622a, 622b of the plurality of wings 621a, 621b. This force may cause the plurality of arms 642a, 642b to flex inward, toward the plunger rod 650, allowing the second housing 620 to move in a proximal direction toward the distal surface of the rear cap 640. In this manner, the plurality of arms 642a, 642b may be bypassed and the second housing 620 may be moved proximally to no longer extend over the imitation needle 631. When the second housing 620 reaches a maximal proximal position, the plurality of arms 642a, 642b of the rear cap may be again received by the windows 623a, 623b of the plurality of wings 621a, 621b of the second housing 620, allowing the plurality of arms 642a, 642b to relax outward, away from the plunger rod 650.

When the second housing 620 reaches a maximum proximal position, and the plurality of arms 642a, 642b return outward, the user may apply a force to the plunger rod 650 to move the plunger rod 650 in a proximal direction. As the plunger rod 650 begins to move proximally, the filleted inner surfaces 647a, 647b of the plurality of tabs 643a, 643b may contact the chamfered proximal surfaces 657a, 657b of the step protrusions 652a, 652b. By applying a force sufficient to overcome a friction between the filleted shape of the filleted inner surfaces 647a, 647b and the sloped shape of the chamfered proximal surfaces 657a, 657b, plurality of tabs 643a, 643b are caused to flex outward, away from the plunger rod 650, allowing for further proximal movement of the plunger rod 650 until it reaches a maximal proximal position. In this manner, the lockout mechanism is bypassed and the training device 600 is ready for reuse.

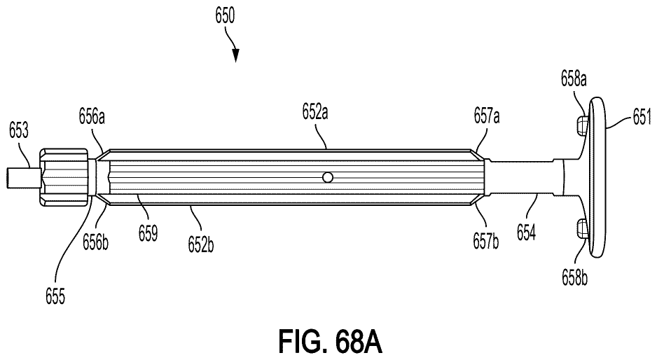

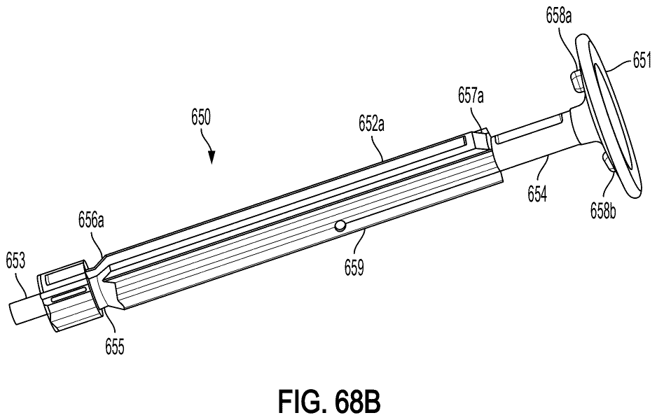

Referring now to FIGS. 68A-68B, the plunger rod 650 may comprise a monolithic or cylindrical body 659 having a proximal end and a distal end opposite its proximal end. At its proximal end, the body 659 may include a proximal portion 654 of the body 659 and a pad 651. The pad 651 may comprise a proximal surface and a distal surface opposite the proximal surface, and may further comprise a plurality of extrusions 658a, 658b on its distal surface. At its distal end, the body 659 may include a distal portion 655 of the body 659 and a cylindrical protrusion 653. The proximal portion 654 and the distal portion 655 may have smaller diameters than the rest of the body 659. The body 659 may further comprise a plurality of step protrusions 652a, 652b having a plurality of chamfered distal surfaces 656a, 656b and a plurality of chamfered proximal surfaces 657a, 657b. The plunger rod 650 may be partially housed within the first housing 610, the second housing 620, and/or the primary container 630. The plunger rod 650 and its various components may interact with other components of the training device 600 as described above. The plurality of extrusions 658a, 658b on the distal surface of the pad 651 may be configured to be received in the plurality of slots 644a, 644b of the collar 641 of the rear cap 640 when the plunger rod 650 is in a maximal distal position.

In some embodiments, plunger rod 650 may not include any indents or notches on a surface of body 659, as depicted in FIGS. 68A-68B. In other embodiments, plunger rod 650 may include one or more indents or notches on a surface of body 659 may include one or more (e.g., one, two, three, four, five, six, seven, eight, or more) indents or notches. The one or more indents may be located on a surface of body 659 corresponding to one or more injection doses that would be delivered by a drug delivery device whose operation is meant to be stimulated by training device 600. For example, a surface of body 659 may include multiple (e.g., two, three, four, five, six, seven, eight, or more) indents, such as the multiple indents 497 of plunger rod 56 as depicted in FIGS. 54A-56B. In some aspects, the one or more indents or notches may include multiple sets of indents at different locations (heights, lengths) along a surface of body 659. For example, the one or more indents or notches may include two indents in a first set on opposite surfaces of body 659 at a first height along body 659, and two indents in a second set on opposite surfaces of body 659 at a second height along body 659, wherein the second height is different from the first height. Further, the multiple sets of indents may be offset in a circumferential direction along body 659, such as being disposed at about 90 degrees offset from one another. According to the present disclosure, the multiple sets of indents may include more than two sets of indents, such as three sets of indents, four sets of indents, five sets of indents, six sets of indents, seven sets of indents, eight sets of indents, or more. It is also contemplated that each set of indents may include more than two indents, such as three indents, four indents, five indents, six indents, seven indents, eight indents, or more. Inclusion of these indents would allow for a user to simulate, with training device 600, the operation of a drug delivery device configured for delivery of multiple different dose amounts of a medicament.

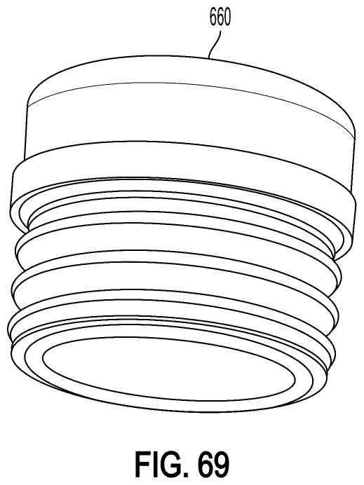

Referring now to FIG. 69, the piston 660 may have a cylindrical shape and may be configured to receive the cylindrical protrusion 653 of the plunger rod 650. The piston 660 may be housed within the primary container 630. In some embodiments, contact between the piston 660 and the primary container 630 may generate a glide force. In some embodiments, the piston 660 may be attached to the cylindrical protrusion 653 via a press-fit, threaded, or adhesive connection. In some embodiments, the piston 660 may be a non-transparent material, such as rubber, while the second housing 620 and the primary container 630 are transparent, such that the piston 660 is visible to a user handling the device 600.

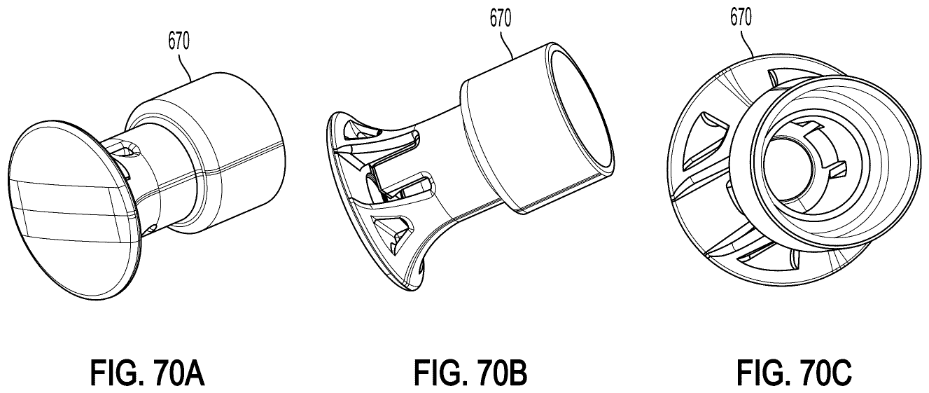

Referring now to FIGS. 70A-70C, the cap 670 may be a single component. The cap 670 may be configured to receive the distal portion 624 of the second housing 620. Alternatively, the cap 670 may be attached to the distal portion 624 of the second housing 620. The cap 670 may be further configured to receive the needle shield 680, which may itself be configured to receive the imitation needle 631, and may be housed within the distal portion 624 of the second housing 620.

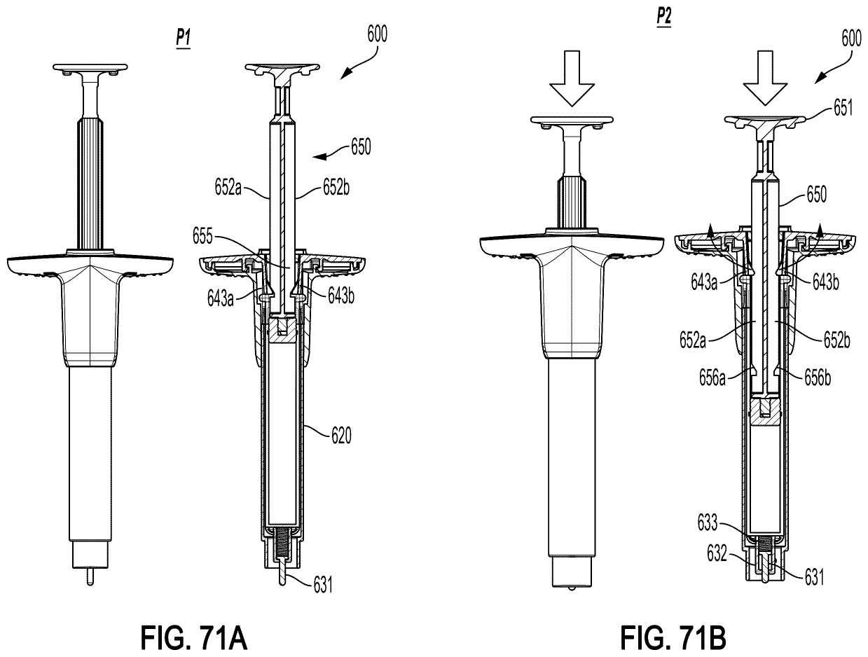

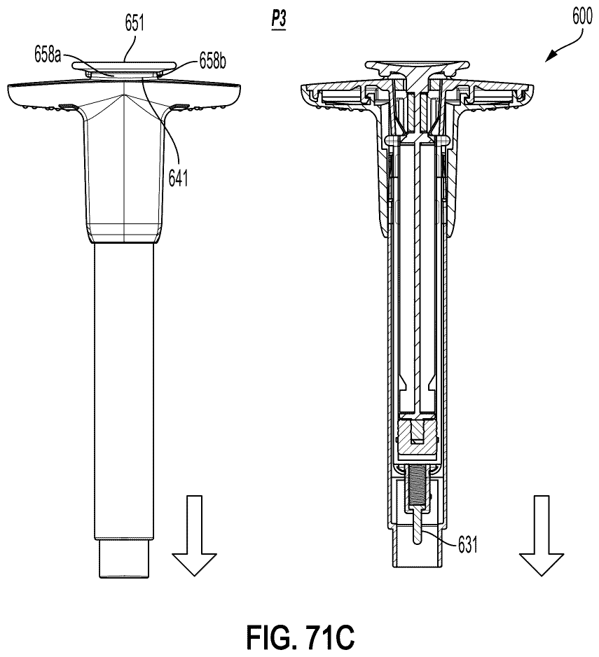

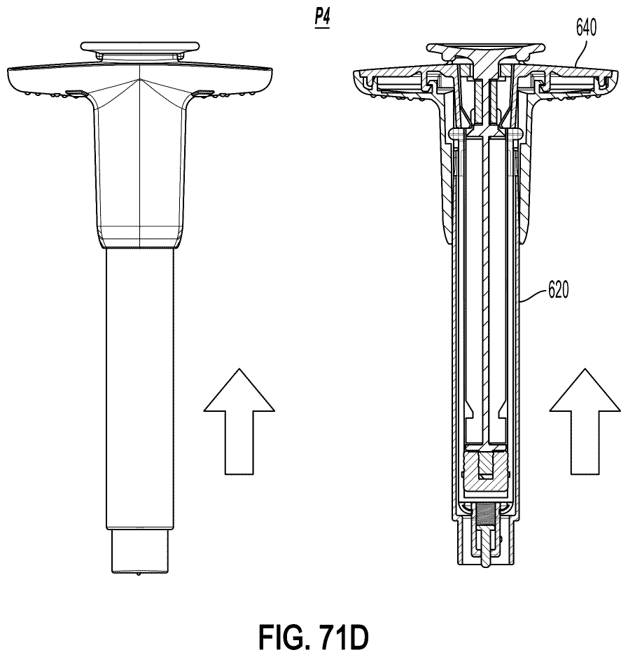

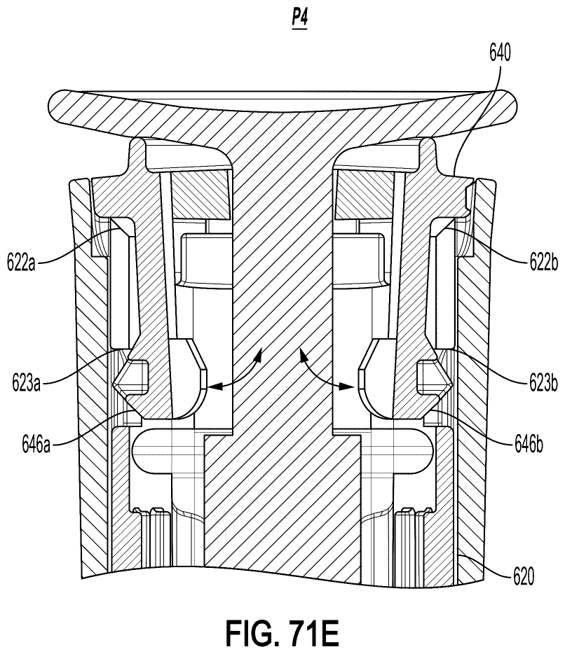

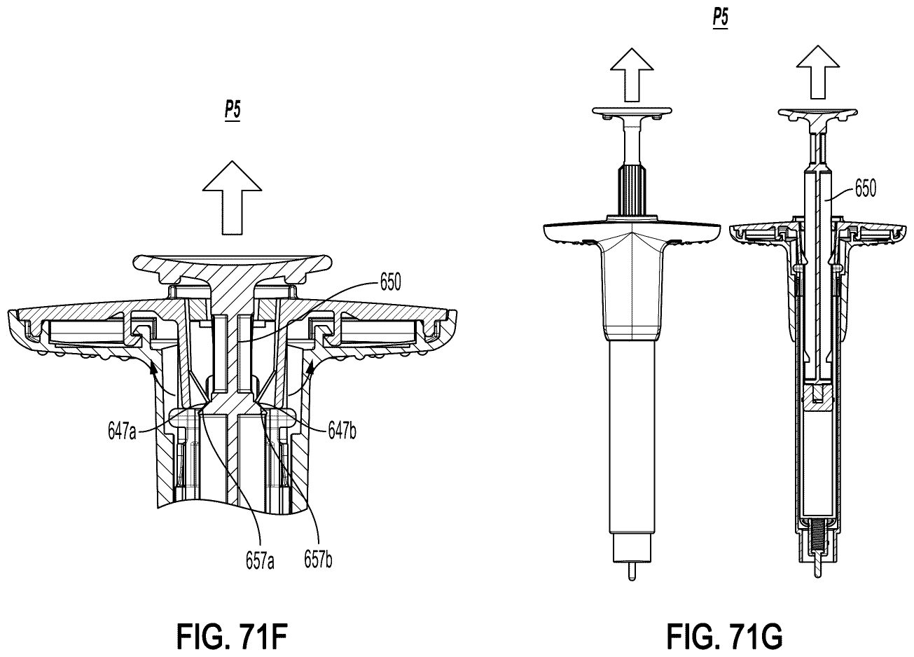

Further provided are methods for using a training device as described herein to simulate injection with a drug delivery device and to reset the device for reuse. An exemplary method for using a training device 600 is provided in FIGS. 71A-71G.

Referring now to FIG. 71A, the training device 600 may begin in a position P1, in which the plunger rod 650 is at a maximal proximal position, and the second housing 620 is at a maximum proximal position. In the P1 position, the plurality of arms 642a, 642b (not shown) may be in contact with body 659 of the plunger rod 650 while the plurality of tabs 643a, 643b of the rear cap 640 may be in contact with the distal portion 655 of the plunger rod 650. This contact may prevent unintended proximal movement and/or distal movement of the plunger rod 650, and may retain the plunger rod 650 in device 600.

Referring now to FIG. 71B, a user may bring the training device 600 into contact with a surface (such as a user's skin), such that the imitation needle 631 contacts the surface. Contact between the imitation needle 631 and the surface may cause a force to be applied to the imitation needle 631 in the proximal direction. Proximal movement of the imitation needle 631 may cause the attached spring 633 to compress, permitting the imitation needle 631 to retract within the tip 632, and simulating the experience of inserting a needle of a drug delivery device. A user may then apply a force to the pad 651 of the plunger rod 650 in the distal direction, to distally move the plunger rod 650. As the plunger rod 650 is moved distally to an intermediate position P2, the chamfered distal surfaces 656a, 656b of the plurality of step protrusions 652a, 652b may contact the plurality of tabs 643a, 643b, applying a force causing the plurality of tabs 643a, 643b to flex outward, away from the plunger rod 650. Outward movement of the plurality of tabs 643a, 643b increases the width of an opening within the device 600, such that the plurality of step protrusions 652a, 652b of the plunger rod 650 are able to pass through the opening, allowing for further distal movement of the plunger rod 650.

Referring now to FIG. 71C, distal movement of the plunger rod 650 may proceed until the plunger rod 650 reaches a maximal distal position P3, when the pad 651 contacts the collar 641 of the rear cap 640. Contact between the pad 651 and the collar 641 may result in a plurality of extrusions 658a, 658b on the pad 651 being received by a plurality of slots 644a, 644b in the collar 641. Alternatively, contact between the pad 651 and the collar 641 may occur in the absence of a plurality of extrusions on the pad 651 and a plurality of slots in the collar 641. As the plunger rod 650 is moved distally towards a maximal distal position P3, the second housing 620 moves distally to cover the imitation needle 631 and allow the length of the imitation needle 631, forcing removal of the imitation needle 631 from the surface and allowing the imitation needle 631 to reset to its original, extended state. At position P3, the training device 600 is in a lockout configuration, such that distal movement and proximal movement of the plunger rod 650 is prevented.

Once in a lockout configuration, the training device 600 may be reset to allow for repeated use of the device 600. Reset may occur only by a specific application of force, or by a specific sequence of applications of force, so as to prevent unintended reset of the device.

Referring now to FIGS. 71D and 71E, a user may apply a force to the second housing 620 in a proximal direction to proximally move the second housing 620 toward the rear cap 640. As shown in FIG. 71D, proximal movement of the second housing 620 may bring the chamfered surfaces 622a, 622b of the plurality of wings 621a, 621b of the second housing 620 into contact with the chamfered outer surfaces 646a, 646b of the plurality of arms 642a, 642b. Contact with the chamfered surfaces 622a, 622b of the plurality of wings 621a, 621b may cause the plurality of arms 642a, 642b to flex inward, toward the plunger rod 650, allowing the second housing 620 to bypass the plurality of arms 642a, 642b and continue moving distally toward the rear cap 640. As shown in FIG. 71E, when the second housing 620 reaches a maximal proximal position, the plurality of arms 642a, 642b are received by the windows 623a, 623b of the plurality of wings 621a, 621b, which permits the plurality of arms 642a, 642b to relax outward, away from the plunger rod 650, anchoring the second housing 620 in place.

Referring now to FIGS. 71F and 71G, after the second housing 620 reaches a maximal proximal position, a user may apply a force to the plunger rod 650 in the proximal direction, to proximally move the plunger rod 650 as shown in intermediate position P5. As shown in FIG. 71F, initial proximal movement of the plunger rod 650 may bring the chamfered proximal surfaces 657a, 657b of the plurality of step protrusions 652a, 652b into contact with the filleted inner surfaces 647a, 647b of the plurality of tabs 643a, 643b. Contact between the chamfered proximal surfaces 657a, 657b of the plurality of step protrusions 652a, 652b and the filleted inner surfaces 647a, 647b of the plurality of tabs 643a, 643b causes the plurality of tabs 643a, 643b to flex outward, away from the plunger rod 650. As shown in FIG. 71G, outward flexion of the plurality of tabs 643a, 643b permits further proximal movement of the plunger rod 650. Proximal movement of the plunger rod 650 may continue until the plunger rod 650 reaches a maximal proximal position, returning the device 600 to its initial position P1. In this configuration, the training device 600 is ready for reuse.

In some embodiments, a method of using a training device as described herein comprises: contacting the primary container 630 to a surface; applying a force to the pad 651 of the plunger rod 650 to distally move the plunger rod 650 until the pad 651 contacts the collar 641 of the rear cap 640 and the plunger rod 650 is in a maximal distal position (P3); applying a force to the second housing 620 to proximally move the second housing 620 until the chamfered proximal surfaces 622a, 622b of the plurality of wings 621a, 621b contact the chamfered outer surfaces 646a, 646b of the plurality of arms 642a, 642b, and the second housing 620 is in a maximal proximal position (P4); applying a force to the pad 651 of the plunger rod 650 to proximally move the plunger rod 650 until a distal portion 655 of the plunger rod 650 contacts the filleted inner surfaces 647a, 647b of the plurality of tabs 643a, 643b and the plunger rod 650 is in a maximal proximal position (P1).

The description above and examples are illustrative and are not intended to be restrictive. One of ordinary skill in the art may make numerous modifications and/or changes without departing from the general scope of the invention. For example, and as has been referenced, aspects of above-described embodiments may be used in any suitable combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description.

The following items illustrate additional embodiments of the present disclosure.

Item 1. A training device comprising:
a first housing;
a second housing partially housed within the first housing, wherein the second housing comprises a proximal end comprising a plurality of wings and a distal end opposite to the proximal end, wherein each of the plurality of wings comprises a chamfered proximal surface;
a primary container housed within the second housing;
a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface; and
a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces.

Item 2. The device of item 1, further comprising a lockout mechanism comprising: a proximal portion of the plunger rod configured to receive the plurality of tabs and contact the filleted inner surfaces of the plurality of tabs when the plunger rod is in a maximal distal position in which the pad of the plunger rod contacts the collar of the rear cap.

Item 3. The device of item 2, wherein contact between the proximal portion of the plunger rod and the filleted inner surfaces of the plurality of tabs prevents proximal movement of the plunger rod.

Item 4. The device of item 1, further comprising a reset mechanism comprising:
the chamfered proximal surfaces of the plurality of wings of the second housing, wherein the chamfered proximal surfaces are configured to contact the chamfered outer surfaces of the plurality of arms and apply a inward-directing force to the plurality of arms when the second housing is in a maximal proximal position, wherein contact between the chamfered proximal surfaces of the plurality of wings and the chamfered outer surfaces of the plurality of arms allow proximal movement of the plunger rod; and
a distal portion of the plunger rod configured to contact the filleted inner surfaces of the plurality of tabs and apply an outward-directing force to the plurality of tabs when the plunger rod is in a maximal proximal position.

Item 5. The device of item 1, wherein the primary container comprises:
a tip;
an imitation needle; and
a spring.

Item 6. The device of item 5, wherein the imitation needle is attached to the spring, the spring is housed in the tip, and the tip is attached to the primary container.

Item 7. The device of item 5, wherein contact between the imitation needle and an external surface causes compression of the spring and retraction of the imitation needle into the tip.

Item 8. The device of item 5, wherein the tip is attached to the primary container via a snap-on connection, threaded connection, or adhesive connection.

Item 9. The device of item 1, further comprising: a cap configured to receive the distal end of the second housing.

Item 10. The device of item 1, wherein the distal end of the plunger rod comprises a cylindrical protrusion attached to a piston housed in the primary container.

Item 11. The device of item 10, wherein contact between the piston and the primary container generates a glide force.

Item 12. The device of item 10, wherein the piston is attached to the cylindrical protrusion via a press-fit, threaded, or adhesive connection.

Item 13. The device of item 1, further comprising: a plurality of extrusions on a distal surface of the pad, and a plurality of slots on the collar of the rear cap.

Item 14. The device of item 13, wherein the plurality of slots of the collar of the rear cap are configured to receive the plurality of extrusions on the distal surface of the pad of the plunger rod.

Item 15. The device of item 13, wherein an interaction between the plurality of extrusions of the pad and the plurality of slots of the collar maintains an alignment of the plunger rod and the rear cap.

Item 16. The device of item 13, wherein the plurality of extrusions of the pad inhibit maximum distal movement of the plunger rod when the plurality of extrusions are not received in the plurality of slots of the collar.

Item 17. The device of item 1, wherein the plunger rod inhibits movement of the second housing when the plunger rod is at a maximum proximal position.

Item 18. The device of item 1, wherein the plurality of tabs inhibit movement of the plunger rod when the plunger rod is at a maximum distal position.

Item 19. The device of item 1, wherein the second housing and/or the primary container are transparent.

Item 20. A method of using the training device of any one of the preceding items, the method comprising:
contacting the primary container to a surface;
applying a force to the pad of the plunger rod to distally move the plunger rod until the pad contacts the collar of the rear cap;
applying a force to the second housing to proximally move the second housing until chamfered proximal surfaces of the plurality of wings contact the chamfered outer surfaces of the plurality of arms and the second housing is in a maximal proximal position; and
applying a force to the pad of the plunger rod to proximally move the plunger rod until a distal portion of the plunger rod contacts the filleted inner surfaces of the plurality of tabs and the plunger rod is in a maximal proximal position.

Item 21. A training device comprising:
a first housing;
a second housing partially housed within the first housing, wherein the second housing comprises a proximal end comprising a plurality of wings and a distal end opposite to the proximal end, wherein each of the plurality of wings comprises a chamfered proximal surface;
a primary container housed within the second housing;
a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface;
a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces;
a lockout mechanism configured to prevent proximal movement of the plunger rod when the plunger rod is in a maximal distal position; and
a reset mechanism configured to allow proximal movement of the plunger rod when the second housing is in a maximal proximal position.

1. A training device comprising:
a first housing;
a second housing partially housed within the first housing, wherein the second housing comprises a proximal end comprising a plurality of wings and a distal end opposite to the proximal end, wherein each of the plurality of wings comprises a chamfered proximal surface;
a primary container housed within the second housing;
a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface; and
a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces.

2. The device of claim 1, further comprising a lockout mechanism comprising:
a proximal portion of the plunger rod configured to receive the plurality of tabs and contact the filleted inner surfaces of the plurality of tabs when the plunger rod is in a maximal distal position in which the pad of the plunger rod contacts the collar of the rear cap.

3. The device of claim 2, wherein contact between the proximal portion of the plunger rod and the filleted inner surfaces of the plurality of tabs prevents proximal movement of the plunger rod.

4. The device of claim 1, further comprising a reset mechanism comprising:
the chamfered proximal surfaces of the plurality of wings of the second housing, wherein the chamfered proximal surfaces are configured to contact the chamfered outer surfaces of the plurality of arms and apply a inward-directing force to the plurality of arms when the second housing is in a maximal proximal position, wherein contact between the chamfered proximal surfaces of the plurality of wings and the chamfered outer surfaces of the plurality of arms allow proximal movement of the plunger rod; and
a distal portion of the plunger rod configured to contact the filleted inner surfaces of the plurality of tabs and apply an outward-directing force to the plurality of tabs when the plunger rod is in a maximal proximal position.

5. The device of claim 1, wherein the primary container comprises:
a tip;
an imitation needle; and
a spring.

6. The device of claim 5, wherein the imitation needle is attached to the spring, the spring is housed in the tip, and the tip is attached to the primary container.

7. The device of claim 5, wherein contact between the imitation needle and an external surface causes compression of the spring and retraction of the imitation needle into the tip.

8. The device of claim 5, wherein the tip is attached to the primary container via a snap-on connection, threaded connection, or adhesive connection.

9. The device of claim 1, further comprising a cap configured to receive the distal end of the second housing.

10. The device of claim 1, wherein the distal end of the plunger rod comprises a cylindrical protrusion attached to a piston housed in the primary container.

11. The device of claim 10, wherein contact between the piston and the primary container generates a glide force.

12. The device of claim 10, wherein the piston is attached to the cylindrical protrusion via a press-fit, threaded, or adhesive connection.

13. The device of claim 1, further comprising:
a plurality of extrusions on a distal surface of the pad, and a plurality of slots on the collar of the rear cap.

14. The device of claim 13, wherein the plurality of slots of the collar of the rear cap are configured to receive the plurality of extrusions on the distal surface of the pad of the plunger rod.

15. The device of claim 13, wherein an interaction between the plurality of extrusions of the pad and the plurality of slots of the collar maintains an alignment of the plunger rod and the rear cap.

16. The device of claim 13, wherein the plurality of extrusions of the pad inhibit maximum distal movement of the plunger rod when the plurality of extrusions are not received in the plurality of slots of the collar.

17. The device of claim 1, wherein the plunger rod inhibits movement of the second housing when the plunger rod is at a maximum proximal position.

18. The device of claim 1, wherein the plurality of tabs inhibit movement of the plunger rod when the plunger rod is at a maximum distal position.

19. The device of claim 1, wherein the second housing and/or the primary container are transparent.

20. A training device comprising:
a first housing;
a second housing partially housed within the first housing, wherein the second housing comprises a proximal end comprising a plurality of wings and a distal end opposite to the proximal end, wherein each of the plurality of wings comprises a chamfered proximal surface;
a primary container housed within the second housing;
a rear cap partially housed within the first housing and the second housing, wherein the rear cap comprises a collar, a plurality of arms, and a plurality of tabs, wherein each of the plurality of arms comprises a chamfered outer surface and each of the plurality of tabs comprises a filleted inner surface;
a plunger rod partially housed within the first housing and the second housing, wherein the plunger rod comprises a body, a proximal end comprising a pad, and a distal end opposite to the proximal end, and wherein the body comprises a plurality of step protrusions having a plurality of chamfered proximal surfaces and a plurality of chamfered distal surfaces;
a lockout mechanism configured to prevent proximal movement of the plunger rod when the plunger rod is in a maximal distal position; and
a reset mechanism configured to allow proximal movement of the plunger rod when the second housing is in a maximal proximal position.
